SEQUENCE LISTING (198 sequences)

SEQ ID NO: 1 - protein, 117 aa, Artificial Sequence
  clone ADI-27705 Heavy chain variable region
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 2 - protein, 107 aa, Artificial Sequence
  clone ADI-27705 light chain variable region
DIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIK

SEQ ID NO: 3 - protein, 9 aa, Artificial Sequence
  CDR1 of clone ADI-27705 - VH
GSFSGYYWS

SEQ ID NO: 4 - protein, 16 aa, Artificial Sequence
  CDR2 of clone ADI-27705 - VH
EIDHSGSTNYNPSLKS

SEQ ID NO: 5 - protein, 11 aa, Artificial Sequence
  CDR3 of clone ADI-27705 - VH
ARARGPWSFDP

SEQ ID NO: 6 - protein, 117 aa, Artificial Sequence; identical to SEQ ID NO: 1
  clone ADI-27724 Heavy chain variable region

SEQ ID NO: 7 - protein, 108 aa, Artificial Sequence
  clone ADI-27724 light chain variable region
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGGGTKVEIK

SEQ ID NO: 8 - protein, 117 aa, Artificial Sequence; identical to SEQ ID NO: 1
  clone ADI-27740 Heavy chain variable region

SEQ ID NO: 9 - protein, 106 aa, Artificial Sequence
  clone ADI-27740 light chain variable region
DIQMTQSPSTLSASVGDRVTITCRASQSIGSWLAWYQQKPGKAPKLLIYKASSLESGVPSRFSGSGSGTEFTLTISSLQPDDFATYYCQQYHSFYTFGGGTKVEIK

SEQ ID NO: 10 - protein, 117 aa, Artificial Sequence; identical to SEQ ID NO: 1
  clone ADI-27741 Heavy chain variable region

SEQ ID NO: 11 - protein, 106 aa, Artificial Sequence
  clone ADI-27741 light chain variable region
DIQMTQSPSTLSASVGDRVTITCRASQSIGSWLAWYQQKPGKAPKLLIYKASSLESGVPSRFSGSGSGTEFTLTISSLQPDDFATYYCQQSNSYYTFGGGTKVEIK

SEQ ID NO: 12 - protein, 117 aa, Artificial Sequence; identical to SEQ ID NO: 1
  clone ADI-27743 Heavy chain variable region

SEQ ID NO: 13 - protein, 106 aa, Artificial Sequence
  clone ADI-27743 Light chain variable region
DIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIK

SEQ ID NO: 14 - protein, 117 aa, Artificial Sequence
  clone ADI-28153 Heavy chain variable region
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWGFDPWGQGTLVTVSS

SEQ ID NO: 15 - protein, 107 aa, Artificial Sequence
  clone ADI-28153 Light chain variable region
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIK

SEQ ID NO: 16 - protein, 117 aa, Artificial Sequence; identical to SEQ ID NO: 1
  clone ADI-28226(C26) Heavy chain variable region

SEQ ID NO: 17 - protein, 107 aa, Artificial Sequence
  clone ADI-28226(C26) Light chain variable region
DIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIK

SEQ ID NO: 18 - protein, 117 aa, Artificial Sequence; identical to SEQ ID NO: 1
  clone ADI-28154 Heavy chain variable region

SEQ ID NO: 19 - protein, 107 aa, Artificial Sequence
  clone ADI-28154 Light chain variable region
DIQMTQSPSTLSASVGDRVTITCRASQSISSWLAWYQQKPGKAPKLLIYKASSLESGVPSRFSGSGSGTDFTLTISSLQPDDFATYYCQQSKEVPWTFGQGTKVEIK

SEQ ID NO: 20 - protein, 117 aa, Artificial Sequence; identical to SEQ ID NO: 1
  clone ADI-29399 Heavy chain variable region

SEQ ID NO: 21 - protein, 106 aa, Artificial Sequence
  clone ADI-29399 Light chain variable region
DIQMTQSPSTLSASVGDRVTITCRASQSISSWLAWYQQKPGKAPKLLIYKASSLESGVPSRFSGSGSGTEFTLTISSLQPDDFATYYCQQYNSFPTFGGGTKVEIK

SEQ ID NO: 22 - protein, 117 aa, Artificial Sequence; identical to SEQ ID NO: 1
  clone ADI-29401 Heavy chain variable region

SEQ ID NO: 23 - protein, 106 aa, Artificial Sequence
  clone ADI-29401 Light chain variable region
DIQMTQSPSTLSASVGDRVTITCRASQSIGSWLAWYQQKPGKAPKLLIYKASSLESGVPSRFSGSGSGTEFTLTISSLQPDDFATYYCQQYDIYPTFGGGTKVEIK

SEQ ID NO: 24 - protein, 117 aa, Artificial Sequence; identical to SEQ ID NO: 1
  clone ADI-29403 Heavy chain variable region

SEQ ID NO: 25 - protein, 106 aa, Artificial Sequence
  clone ADI-29403 Light chain variable region
DIQMTQSPSTLSASVGDRVTITCRASQSISSWLAWYQQKPGKAPKLLIYKASSLESGVPSRFSGSGSGTEFTLTISSLQPDDFATYYCQQYDSYPTFGGGTKVEIK

SEQ ID NO: 26 - protein, 117 aa, Artificial Sequence; identical to SEQ ID NO: 1
  clone ADI-29405 Heavy chain variable region

SEQ ID NO: 27 - protein, 106 aa, Artificial Sequence
  clone ADI-29405 Light chain variable region
DIQMTQSPSTLSASVGDRVTITCRASQSISSWLAWYQQKPGKAPKLLIYKASSLESGVPSRFSGSGSGTEFTLTISSLQPDDFATYYCQQYGSFPTFGGGTKVEIK

SEQ ID NO: 28 - protein, 117 aa, Artificial Sequence; identical to SEQ ID NO: 1
  clone ADI-29407 Heavy chain variable region

SEQ ID NO: 29 - protein, 106 aa, Artificial Sequence
  clone ADI-29407 Light chain variable region
DIQMTQSPSTLSASVGDRVTITCRASQSISSWLAWYQQKPGKAPKLLIYKASSLESGVPSRFSGSGSGTEFTLTISSLQPDDFATYYCQQYQSFPTFGGGTKVEIK

SEQ ID NO: 30 - protein, 117 aa, Artificial Sequence; identical to SEQ ID NO: 1
  clone ADI-29419 Heavy chain variable region

SEQ ID NO: 31 - protein, 106 aa, Artificial Sequence
  clone ADI-29419 Light chain variable region
DIQMTQSPSTLSASVGDRVTITCRASQSISSWLAWYQQKPGKAPKLLIYKASSLESGVPSRFSGSGSGTEFTLTISSLQPDDFATYYCQQYSSFSTFGGGTKVEIK

SEQ ID NO: 32 - protein, 117 aa, Artificial Sequence; identical to SEQ ID NO: 1
  clone ADI-29421 Heavy chain variable region

SEQ ID NO: 33 - protein, 106 aa, Artificial Sequence
  clone ADI-29421 Light chain variable region
DIQMTQSPSTLSASVGDRVTITCRASQSISSWLAWYQQKPGKAPKLLIYKASSLESGVPSRFSGSGSGTEFTLTISSLQPDDFATYYCQQYESYSTFGGGTKVEIK

SEQ ID NO: 34 - protein, 117 aa, Artificial Sequence; identical to SEQ ID NO: 1
  clone ADI-29424 Heavy chain variable region

SEQ ID NO: 35 - protein, 106 aa, Artificial Sequence
  clone ADI-29424 Light chain variable region
DIQMTQSPSTLSASVGDRVTITCRASQSISSWLAWYQQKPGKAPKLLIYKASSLESGVPSRFSGSGSGTEFTLTISSLQPDDFATYYCQQYDSFITFGGGTKVEIK

SEQ ID NO: 36 - protein, 117 aa, Artificial Sequence; identical to SEQ ID NO: 1
  clone ADI-29425 Heavy chain variable region

SEQ ID NO: 37 - protein, 106 aa, Artificial Sequence
  clone ADI-29425 Light chain variable region
DIQMTQSPSTLSASVGDRVTITCRASQSISSWLAWYQQKPGKAPKLLIYKASSLESGVPSRFSGSGSGTEFTLTISSLQPDDFATYYCQQYQSYPTFGGGTKVEIK

SEQ ID NO: 38 - protein, 117 aa, Artificial Sequence; identical to SEQ ID NO: 1
  clone ADI-29426 Heavy chain variable region

SEQ ID NO: 39 - protein, 106 aa, Artificial Sequence
  clone ADI-29426 Light chain variable region
DIQMTQSPSTLSASVGDRVTITCRASQSIGSWLAWYQQKPGKAPKLLIYKASSLESGVPSRFSGSGSGTEFTLTISSLQPDDFATYYCQQYHSFPTFGGGTKVEIK

SEQ ID NO: 40 - protein, 117 aa, Artificial Sequence; identical to SEQ ID NO: 1
  clone ADI-29429 Heavy chain variable region

SEQ ID NO: 41 - protein, 107 aa, Artificial Sequence
  clone ADI-29429 Light chain variable region
DIQMTQSPSTLSASVGDRVTITCRASQSIGSWLAWYQQKPGKAPKLLIYKASSLESGVPSRFSGSGSGTEFTLTISSLQPDDFATYYCQQYELYSYTFGGGTKVEIK

SEQ ID NO: 42 - protein, 117 aa, Artificial Sequence; identical to SEQ ID NO: 1
  clone ADI-29447(F47) Heavy chain variable region

SEQ ID NO: 43 - protein, 106 aa, Artificial Sequence
  clone ADI-29447(F47) Light chain variable region
DIQMTQSPSTLSASVGDRVTITCRASQSISSWLAWYQQKPGKAPKLLIYKASSLESGVPSRFSGSGSGTEFTLTISSLQPDDFATYYCQQYDTFITFGGGTKVEIK

SEQ ID NO: 44 - protein, 125 aa, Artificial Sequence
  clone ADI-27727 Heavy chain variable region
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGDSSIRHAYYYYGMDVWGQGTTVTVSS

SEQ ID NO: 45 - protein, 9 aa, Artificial Sequence
  CDR1 of clone ADI-27727 - VH (non-Kabat)
GTFSSYAIS

SEQ ID NO: 46 - protein, 17 aa, Artificial Sequence
  CDR2 of clone ADI-27727 - VH
GIIPIFGTANYAQKFQG

SEQ ID NO: 47 - protein, 18 aa, Artificial Sequence
  CDR3 of clone ADI-27727 - VH (non-Kabat)
ARGDSSIRHAYYYYGMDV

SEQ ID NO: 48 - protein, 113 aa, Artificial Sequence
  clone ADI-27727 Light chain variable region
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPITFGGGTKVEIK

SEQ ID NO: 49 - protein, 17 aa, Artificial Sequence
  CDR1 of clone ADI-27727 - VL
KSSQSVLYSSNNKNYLA

SEQ ID NO: 50 - protein, 7 aa, Artificial Sequence
  CDR2 of clone ADI-27727 - VL
WASTRES

SEQ ID NO: 51 - protein, 9 aa, Artificial Sequence
  CDR3 of clone ADI-27727 - VL
QQYYSTPIT

SEQ ID NO: 52 - protein, 121 aa, Artificial Sequence
  clone ADI-29443(F43) Heavy chain variable region
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARGSDRFHPYFDYWGQGTLVTVSS

SEQ ID NO: 53 - protein, 11 aa, Artificial Sequence
  CDR1 of clone ADI-29443(F43)- VH (non-Kabat)
GSISSSSYYWG

SEQ ID NO: 54 - protein, 16 aa, Artificial Sequence
  CDR2 of clone ADI-29443(F43)- VH
SIYYSGSTYYNPSLKS

SEQ ID NO: 55 - protein, 13 aa, Artificial Sequence
  CDR3 of clone ADI-29443(F43)- VH (non-Kabat)
ARGSDRFHPYFDY

SEQ ID NO: 56 - protein, 107 aa, Artificial Sequence
  clone ADI-29443(F43) Light chain variable region
EIVLTQSPATLSLSPGERATLSCRASQSVSRYLAWYQQKPGQAPRLLIYDASNRATGIPARFSGSGSGTDFTLTISSLEPEDFAVYYCQQFDTWPPTFGGGTKVEIK

SEQ ID NO: 57 - protein, 11 aa, Artificial Sequence
  CDR1 of clone ADI-29443(F43)- VL
RASQSVSRYLA

SEQ ID NO: 58 - protein, 7 aa, Artificial Sequence
  CDR2 of clone ADI-29443(F43)- VL
DASNRAT

SEQ ID NO: 59 - protein, 9 aa, Artificial Sequence
  CDR3 of clone ADI-29443(F43)- VL
QQFDTWPPT

SEQ ID NO: 60 - protein, 117 aa, Artificial Sequence; identical to SEQ ID NO: 1
  clone ADI-29404(F04) Heavy chain variable region

SEQ ID NO: 61 - protein, 106 aa, Artificial Sequence
  clone ADI-29404(F04) Light chain variable region
DIQMTQSPSTLSASVGDRVTITCRASQSISSWLAWYQQKPGKAPKLLIYKASSLESGVPSRFSGSGSGTEFTLTISSLQPDDFATYYCEQYDSYPTFGGGTKVEIK

SEQ ID NO: 62 - protein, 126 aa, Artificial Sequence
  clone ADI-28200 Heavy chain variable region
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRGRKASGSFYYYYGMDVWGQGTTVTVSS

SEQ ID NO: 63 - protein, 9 aa, Artificial Sequence
  CDR1 of clone ADI-28200 - VH (non-Kabat)
GTFSSYAIS

SEQ ID NO: 64 - protein, 17 aa, Artificial Sequence
  CDR2 of clone ADI-28200 - VH
GIIPIFGTANYAQKFQG

SEQ ID NO: 65 - protein, 19 aa, Artificial Sequence
  CDR3 of clone ADI-28200 - VH
ARRGRKASGSFYYYYGMDV

SEQ ID NO: 66 - protein, 113 aa, Artificial Sequence [sequence omitted]

SEQ ID NO: 67 - protein, 17 aa, Artificial Sequence
  CDR1 of clone ADI-28200 - VL
ESSQSLLNSGNQKNYLT

SEQ ID NO: 68 - protein, 7 aa, Artificial Sequence
  CDR2 of clone ADI-28200 - VL
WASTRES

SEQ ID NO: 69 - protein, 9 aa, Artificial Sequence
  CDR3 of clone ADI-28200 - VL
QNDYSYPYT

SEQ ID NO: 70 - protein, 126 aa, Artificial Sequence
  clone ADI-29379(E79) Heavy chain variable region
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGAPNYGDTTHDYYYMDVWGKGTTVTVSS

SEQ ID NO: 71 - protein, 9 aa, Artificial Sequence
  CDR1 of clone ADI-29379(E79) - VH (non-Kabat)
YTFTSYYMH

SEQ ID NO: 72 - protein, 17 aa, Artificial Sequence
  CDR2 of clone ADI-29379(E79) - VH
IINPSGGSTSYAQKFQG

SEQ ID NO: 73 - protein, 19 aa, Artificial Sequence
  CDR3 of clone ADI-29379(E79) - VH (non-Kabat)
ARGAPNYGDTTHDYYYMDV

SEQ ID NO: 74 - protein, 107 aa, Artificial Sequence
  clone ADI-29379(E79) Light chain variable region
EIVMTQSPATLSVSPGERATLSCRASQSVSSNLAWYQQKPGQAPRLLIYGASTRATGIPARFSGSGSGTEFTLTISSLQSEDFAVYYCQQYDDWPFTFGGGTKVEIK

SEQ ID NO: 75 - protein, 11 aa, Artificial Sequence
  CDR1 of clone ADI-29379(E79) - VL
RASQSVSSNLA

SEQ ID NO: 76 - protein, 7 aa, Artificial Sequence
  CDR2 of clone ADI-29379(E79) - VL
GASTRAT

SEQ ID NO: 77 - protein, 9 aa, Artificial Sequence
  CDR3 of clone ADI-29379(E79) - VL
QQYDDWPFT

SEQ ID NO: 78 - protein, 124 aa, Artificial Sequence
  clone ADI-29463(F63) Heavy chain variable region
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDTGEYYDTDDHGMDVWGQGTTVTVSS

SEQ ID NO: 79 - protein, 9 aa, Artificial Sequence
  CDR1 of clone ADI-29463(F63) - VH (non-Kabat)
YTFTGYYMH

SEQ ID NO: 80 - protein, 17 aa, Artificial Sequence
  CDR2 of clone ADI-29463(F63) - VH
WINPNSGGTNYAQKFQG

SEQ ID NO: 81 - protein, 17 aa, Artificial Sequence
  CDR3 of clone ADI-29463(F63) - VH
ARDTGEYYDTDDHGMDV

SEQ ID NO: 82 - protein, 107 aa, Artificial Sequence
  clone ADI-29463(F63) Light chain variable region
EIVLTQSPGTLSLSPGERATLSCRASQSVSSNLAWYQQKPGQAPRLLIYGASTRATGIPARFSGSGSGTEFTLTISSLQSEDFAVYYCQQDDYWPPTFGGGTKVEIK

SEQ ID NO: 83 - protein, 11 aa, Artificial Sequence
  CDR1 of clone ADI-29463(F63) - VL
RASQSVSSNLA

SEQ ID NO: 84 - protein, 7 aa, Artificial Sequence
  CDR2 of clone ADI-29463(F63) - VL
GASTRAT

SEQ ID NO: 85 - protein, 9 aa, Artificial Sequence
  CDR3 of clone ADI-29463(F63) - VL
QQDDYWPPT

SEQ ID NO: 86 - protein, 121 aa, Artificial Sequence
  clone ADI-27744(A44) Heavy chain variable region
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYDSGAGDYWGQGTLVTVSS

SEQ ID NO: 87 - protein, 9 aa, Artificial Sequence
  CDR1 of clone ADI-27744(A44) - VH (non-Kabat)
FTFSSYAMS

SEQ ID NO: 88 - protein, 17 aa, Artificial Sequence
  CDR2 of clone ADI-27744(A44) - VH
AISGSGGSTYYADSVKG

SEQ ID NO: 89 - protein, 14 aa, Artificial Sequence
  CDR3 of clone ADI-27744(A44) - VH (non-Kabat)
AKDGGYYDSGAGDY

SEQ ID NO: 90 - protein, 107 aa, Artificial Sequence
  clone ADI-27744(A44) Light chain variable region
DIQMTQSPSSVSASVGDRVTITCRASQGIDSWLAWYQQKPGKAPKLLIYAASSLQSGVPSRFSGSGSGTDFTLTISSLQPEDFATYYCQQGVSYPRTFGGGTKVEIK

SEQ ID NO: 91 - protein, 11 aa, Artificial Sequence
  CDR1 of clone ADI-27744(A44) - VL
RASQGIDSWLA

SEQ ID NO: 92 - protein, 7 aa, Artificial Sequence
  CDR2 of clone ADI-27744(A44) - VL
AASSLQS

SEQ ID NO: 93 - protein, 9 aa, Artificial Sequence
  CDR3 of clone ADI-27744(A44) - VL
QQGVSYPRT

SEQ ID NO: 94 - protein, 122 aa, Artificial Sequence
  clone ADI-27749(A49) Heavy chain variable region
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSS

SEQ ID NO: 95 - protein, 9 aa, Artificial Sequence
  CDR1 of clones ADI-27744(A44), A49MI, A49MQ, A49ML, A49MF, A49MV - VH (non-Kabat)
FTFSSYSMN

SEQ ID NO: 96 - protein, 17 aa, Artificial Sequence
  CDR2 of clones ADI-27744(A44), A49MI, A49MQ, A49ML, A49MF, A49MV - VH
SISSSSSYIYYADSVKG

SEQ ID NO: 97 - protein, 15 aa, Artificial Sequence
  CDR3 of clone ADI-27744(A44) - VH (non-Kabat)
ARGAPMGAAAGWFDP

SEQ ID NO: 98 - protein, 107 aa, Artificial Sequence
  clones ADI-27749(A49), A49MQ, A49MF, A49MV, A49-consensus Light chain variable region
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIK

SEQ ID NO: 99 - protein, 11 aa, Artificial Sequence
  CDR1 of clones ADI-27744(A44), A49MI, A49MQ, A49ML, A49MF, A49MV, A49-consensus - VL
RASQGISSWLA

SEQ ID NO: 100 - protein, 7 aa, Artificial Sequence
  CDR2 of clones ADI-27744(A44), A49MI, A49MQ, A49ML, A49MF, A49MV, A49-consensus - VL
AASSLQS

SEQ ID NO: 101 - protein, 9 aa, Artificial Sequence
  CDR3 of clones ADI-27744(A44), A49MI, A49MQ, A49ML, A49MF, A49MV, A49-consensus - VL
QQGVSFPRT

SEQ ID NO: 102 - protein, 125 aa, Artificial Sequence
  clone ADI-29378(E78) Heavy chain variable region
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREGAGFAYGMDYYYMDVWGKGTTVTVSS

SEQ ID NO: 103 - protein, 9 aa, Artificial Sequence
  CDR1 of clone ADI-29378(E78) - VH (non-Kabat)
YTFTSYYMH

SEQ ID NO: 104 - protein, 17 aa, Artificial Sequence
  CDR2 of clone ADI-29378(E78) - VH
IINPSGGSTSYAQKFQG

SEQ ID NO: 105 - protein, 18 aa, Artificial Sequence
  CDR3 of clone ADI-29378(E78) - VH (non-Kabat)
AREGAGFAYGMDYYYMDV

SEQ ID NO: 106 - protein, 107 aa, Artificial Sequence
  clone ADI-29378(E78) Light chain variable region
EIVLTQSPATLSLSPGERATLSCRASQSVSSYLAWYQQKPGQAPRLLIYDASNRATGIPARFSGSGSGTDFTLTISSLEPEDFAVYYCQQSDNWPFTFGGGTKVEIK

SEQ ID NO: 107 - protein, 11 aa, Artificial Sequence
  CDR1 of clone ADI-29378(E78) - VL
RASQSVSSYLA

SEQ ID NO: 108 - protein, 7 aa, Artificial Sequence
  CDR2 of clone ADI-29378(E78) - VL
DASNRAT

SEQ ID NO: 109 - protein, 9 aa, Artificial Sequence
  CDR3 of clone ADI-29378(E78) - VL
QQSDNWPFT

SEQ ID NO: 110 - protein, 121 aa, Artificial Sequence
  heavy chain variable domain
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 111 - protein, 110 aa, Artificial Sequence
  light chain variable domain
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 112 - protein, 115 aa, Artificial Sequence
  heavy chain variable domain
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSS

SEQ ID NO: 113 - protein, 108 aa, Artificial Sequence
  light chain variable domain
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 114 - protein, 119 aa, Artificial Sequence
  clone no. 1 in US14/776649 Heavy chain variable domain
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSS

SEQ ID NO: 115 - protein, 5 aa, Artificial Sequence
  CDR1 of clone no. 1 in US14/776649 - VH
DYYIN

SEQ ID NO: 116 - protein, 17 aa, Artificial Sequence
  CDR2 of clone no. 1 in US14/776649 - VH
WIYFASGNSEYNQKFTG

SEQ ID NO: 117 - protein, 10 aa, Artificial Sequence
  CDR3 of clone no. 1 in US14/776649 - VH
LYDYDWYFDV

SEQ ID NO: 118 - protein, 112 aa, Artificial Sequence
  clone no. 1 in US14/776649 Light chain variable domain
DIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIK

SEQ ID NO: 119 - protein, 112 aa, Artificial Sequence
  clone no. 1 in US14/776649 Light chain variable domain
DIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIK

SEQ ID NO: 120 - protein, 16 aa, Artificial Sequence
  CDR1 of clone no. 1 in US14/776649 - VL
KSSQSLVHSNGNTYLH

SEQ ID NO: 121 - protein, 7 aa, Artificial Sequence
  CDR2 of clone no. 1 in US14/776649 - VL
KVSNRFS

SEQ ID NO: 122 - protein, 9 aa, Artificial Sequence
  CDR3 of clone no. 1 in US14/776649 - VL
AETSHVPWT

SEQ ID NO: 123 - protein, 9 aa, Artificial Sequence
  CDR3 of clone no. 1 in US14/776649 - VL
SQSSIYPWT

SEQ ID NO: 124 - protein, 117 aa, Artificial Sequence
  clone no. 2 in PCT/US15/64269 Heavy chain variable domain
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 125 - protein, 5 aa, Artificial Sequence
  CDR1 of clone no. 2 in PCT/US15/64269 - VH
DYSIN

SEQ ID NO: 126 - protein, 16 aa, Artificial Sequence
  CDR2 of clone no. 2 in PCT/US15/64269 - VH
WINTETREPAYAYDFR

SEQ ID NO: 127 - protein, 8 aa, Artificial Sequence
  CDR3 of clone no. 2 in PCT/US15/64269 - VH
DYSYAMDY

SEQ ID NO: 128 - protein, 111 aa, Artificial Sequence
  clone no. 2 in PCT/US15/64269 Light chain variable domain
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 129 - protein, 15 aa, Artificial Sequence
  CDR1 of clone no. 2 in PCT/US15/64269 - VL
RASESVTILGSHLIH

SEQ ID NO: 130 - protein, 7 aa, Artificial Sequence
  CDR2 of clone no. 2 in PCT/US15/64269 - VL
LASNVQT

SEQ ID NO: 131 - protein, 9 aa, Artificial Sequence
  CDR3 of clone no. 2 in PCT/US15/64269 - VL
LQSRTIPRT

SEQ ID NO: 132 - protein, 121 aa, Artificial Sequence
  clone no. 3 in US14/122,391 Heavy chain variable domain
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSS

SEQ ID NO: 133 - protein, 5 aa, Artificial Sequence
  CDR1 of clone no. 3 in US14/122,391 - VH
NYWMH

SEQ ID NO: 134 - protein, 17 aa, Artificial Sequence
  CDR2 of clone no. 3 in US14/122,391 - VH
ATYRGHSDTYYNQKFKG

SEQ ID NO: 135 - protein, 12 aa, Artificial Sequence
  CDR3 of clone no. 3 in US14/122,391 - VH
GAIYNGYDVLDN

SEQ ID NO: 136 - protein, 108 aa, Artificial Sequence
  clone no. 3 in US14/122,391 Light chain variable domain
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 137 - protein, 11 aa, Artificial Sequence
  CDR1 of clone no. 3 in US14/122,391 - VL
SASQDISNYLN

SEQ ID NO: 138 - protein, 7 aa, Artificial Sequence
  CDR2 of clone no. 3 in US14/122,391 - VL
YTSNLHS

SEQ ID NO: 139 - protein, 9 aa, Artificial Sequence
  CDR3 of clone no. 3 in US14/122,391 - VL
QQYRKLPWT

SEQ ID NO: 140 - protein, 121 aa, Artificial Sequence
  clone no. 4 in US20170051068 Heavy chain variable domain
QLQLQESGPGLVKPSETLSLTCTVSGGSISSSSYFWGWIRQPPGKGLEWIGSIYYSGITYYNPSLKSRVTISVDTSKNQFSLKLSSVTAADTAVYYCARHDGATAGLFDYWGQGTLVTVSS

SEQ ID NO: 141 - protein, 7 aa, Artificial Sequence
  CDR1 of clone no. 4 in US20170051068 - VH
SSSYFWG

SEQ ID NO: 142 - protein, 16 aa, Artificial Sequence
  CDR2 of clone no. 4 in US20170051068 - VH
SIYYSGITYYNPSLKS

SEQ ID NO: 143 - protein, 11 aa, Artificial Sequence
  CDR3 of clone no. 4 in US20170051068 - VH
HDGATAGLFDY

SEQ ID NO: 144 - protein, 108 aa, Artificial Sequence
  clone no. 4 in US20170051068 Light chain variable domain
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVL

SEQ ID NO: 145 - protein, 11 aa, Artificial Sequence
  CDR1 of clone no. 4 in US20170051068 - VL
GGNNIGSKSVH

SEQ ID NO: 146 - protein, 7 aa, Artificial Sequence
  CDR2 of clone no. 4 in US20170051068 - VL
DDSDRPS

SEQ ID NO: 147 - protein, 11 aa, Artificial Sequence
  CDR3 of clone no. 4 in US20170051068 - VL
QVWDSSSDHVV

SEQ ID NO: 148 - protein, 116 aa, Artificial Sequence
  clone no. 5(Mab42) in WO2017021450 Heavy chain variable domain
EVQLLESGGGLVQPGGSLRLSCAASGFTFSDNAMGWVRQAPGKGLEWVSAISGPGSSTYYADSVKGRFTISRDNSKNTLYLQMNSLRAEDTAVYYCAKVLGWFDYWGQGTLVTVSS

SEQ ID NO: 149 - protein, 5 aa, Artificial Sequence
  CDR1 of clone no. 5(Mab42) in WO2017021450 - VH
DNAMG

SEQ ID NO: 150 - protein, 17 aa, Artificial Sequence
  CDR2 of clone no. 5(Mab42) in WO2017021450 - VH
AISGPGSSTYYADSVKG

SEQ ID NO: 151 - protein, 7 aa, Artificial Sequence
  CDR3 of clone no. 5(Mab42) in WO2017021450 - VH
VLGWFDY

SEQ ID NO: 152 - protein, 109 aa, Artificial Sequence
  clone no. 5(Mab42) in WO2017021450 Light chain variable domain
EIVLTQSPGTLSLSPGERATLSCRASQSVSDEYLSWYQQKPGQAPRLLIHSASTRATGIPDRFSGSGSGTDFTLAISRLEPEDFAVYYCQQYGYPPDFTFGQGTKVEIK

SEQ ID NO: 153 - protein, 13 aa, Artificial Sequence
  CDR1 of clone no. 5(Mab42) in WO2017021450 - VL
RASQSVSDEYLSW

SEQ ID NO: 154 - protein, 8 aa, Artificial Sequence
  CDR2 of clone no. 5(Mab42) in WO2017021450 - VL
HSASTRAT

SEQ ID NO: 155 - protein, 10 aa, Artificial Sequence
  CDR3 of clone no. 5(Mab42) in WO2017021450 - VL
QQYGYPPDFT

SEQ ID NO: 156 - protein, 184 aa, Artificial Sequence
  a BCMA-binding domain
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 157 - protein, 116 aa, Artificial Sequence
  83A10 heavy chain variable domain
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVLGWFDYWGQGTLVTVSS

SEQ ID NO: 158 - protein, 109 aa, Artificial Sequence
  83A10 light chain variable domain
EIVLTQSPGTLSLSPGERATLSCRASQSVSSSYLAWYQQKPGQAPRLLIYGASSRATGIPDRFSGSGSGTDFTLTISRLEPEDFAVYYCQQYGYPPDFTFGQGTKVEIK

SEQ ID NO: 159 - protein, 17 aa, Artificial Sequence
  CDR3 of clone ADI-29379(E79) - VH
GAPNYGDTTHDYYYMDV

SEQ ID NO: 160 - protein, 13 aa, Artificial Sequence
  CDR3 of clone A49-consensus - VH
  misc_feature (4)..(4): Xaa = Met, Leu, Ile, Val, Gln or Phe
GAPXGAAAGWFDP

SEQ ID NO: 161 - protein, 249 aa, Artificial Sequence
  NKG2D-binding scFv
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGCGTKVEIKGGGGSGGGGSGGGGSGGGGSEVQLVESGGGLVKPGGSLRLSCAASGFTFSSYSMNWVRQAPGKCLEWVSSISSSSSYIYYADSVKGRFTISRDNAKNSLYLQMNSLRAEDTAVYYCARGAPMGAAAGWFDPWGQGTLVTVSS

SEQ ID NO: 162 - protein, 702 aa, Artificial Sequence
  C-terminus of the Fc domain (F4-BCMAFc- AJchainB-NKG2D-binding scFv)
EVQLLESGGGLVQPGGSLRLSCAASGFTFSDNAMGWVRQAPGKGLEWVSAISGPGSSTYYADSVKGRFTISRDNSKNTLYLQMNSLRAEDTAVYYCAKVLGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPGSGSGGGGSDIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGCGTKVEIKGGGGSGGGGSGGGGSGGGGSEVQLVESGGGLVKPGGSLRLSCAASGFTFSSYSMNWVRQAPGKCLEWVSSISSSSSYIYYADSVKGRFTISRDNAKNSLYLQMNSLRAEDTAVYYCARGAPMGAAAGWFDPWGQGTLVTVSS

SEQ ID NO: 163 - protein, 445 aa, Artificial Sequence
  Anti-BCMA VH-CH1-Fc
EVQLLESGGGLVQPGGSLRLSCAASGFTFSDNAMGWVRQAPGKGLEWVSAISGPGSSTYYADSVKGRFTISRDNSKNTLYLQMNSLRAEDTAVYYCAKVLGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 164 - protein, 20 aa, Artificial Sequence
  linker sequence
GGGGSGGGGSGGGGSGGGGS

SEQ ID NO: 165 - protein, 216 aa, Artificial Sequence
  Anti-BCMA-Whole LC
EIVLTQSPGTLSLSPGERATLSCRASQSVSDEYLSWYQQKPGQAPRLLIHSASTRATGIPDRFSGSGSGTDFTLAISRLEPEDFAVYYCQQYGYPPDFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - protein, 477 aa, Artificial Sequence
  F3- NKG2D-binding scFv-Fc-AJchainB [VL(G4S)4VH)]
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGCGTKVEIKGGGGSGGGGSGGGGSGGGGSEVQLVESGGGLVKPGGSLRLSCAASGFTFSSYSMNWVRQAPGKCLEWVSSISSSSSYIYYADSVKGRFTISRDNAKNSLYLQMNSLRAEDTAVYYCARGAPMGAAAGWFDPWGQGTLVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 167 - protein, 20 aa, Artificial Sequence
  linker sequence
GGGGSGGGGSGGGGSGGGGS

SEQ ID NO: 168 - protein, 8 aa, Artificial Sequence
  linker sequence
SGSGGGGS

SEQ ID NO: 169 - protein, 122 aa, Artificial Sequence
  clone A49MI Heavy Chain variable region
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSS

SEQ ID NO: 170 - protein, 15 aa, Artificial Sequence
  CDR3 of clone A49MI - VH (non-Kabat)
ARGAPIGAAAGWFDP

SEQ ID NO: 171 - protein, 122 aa, Artificial Sequence
  clone A49MQ Heavy chain variable region
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPQGAAAGWFDPWGQGTLVTVSS

SEQ ID NO: 172 - protein, 15 aa, Artificial Sequence
  CDR3 of clone A49MQ - VH (non-Kabat)
ARGAPQGAAAGWFDP

SEQ ID NO: 173 - protein, 122 aa, Artificial Sequence
  clone A49ML Heavy chain variable region
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPLGAAAGWFDPWGQGTLVTVSS

SEQ ID NO: 174 - protein, 15 aa, Artificial Sequence
  CDR3 of clone A49ML - VH (non-Kabat)
ARGAPLGAAAGWFDP

SEQ ID NO: 175 - protein, 122 aa, Artificial Sequence
  clone A49MF Heavy chain variable region
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPFGAAAGWFDPWGQGTLVTVSS

SEQ ID NO: 176 - protein, 15 aa, Artificial Sequence
  CDR3 of Clone A49MF - VH (non-Kabat)
ARGAPFGAAAGWFDP

SEQ ID NO: 177 - protein, 122 aa, Artificial Sequence
  clone A49MV Heavy chain variable region
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPVGAAAGWFDPWGQGTLVTVSS

SEQ ID NO: 178 - protein, 15 aa, Artificial Sequence
  CDR3 of clone A49MV - VH (non-Kabat)
ARGAPVGAAAGWFDP

SEQ ID NO: 179 - protein, 122 aa, Artificial Sequence
  clone A49-consensus Heavy chain variable region
  misc_feature (102)..(102): Xaa can be any naturally occurring amino acid
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPXGAAAGWFDPWGQGTLVTVSS

SEQ ID NO: 180 - protein, 15 aa, Artificial Sequence
  CDR3 of clone A49-consensus - VH (non-Kabat)
  misc_feature (6)..(6): Xaa = Met, Leu, Ile, Val, Gln or Phe
ARGAPXGAAAGWFDP

SEQ ID NO: 181 - protein, 5 aa, Artificial Sequence
  CDR1 of clones ADI-27727, 28200 - VH
SYAIS

SEQ ID NO: 182 - protein, 16 aa, Artificial Sequence
  CDR3 of clone ADI-27727 - VH
GDSSIRHAYYYYGMDV

SEQ ID NO: 183 - protein, 7 aa, Artificial Sequence
  CDR1 of clone ADI-29443(F43)- VH
SSSYYWG

SEQ ID NO: 184 - protein, 11 aa, Artificial Sequence
  CDR3 of clone ADI-29443(F43)- VH
GSDRFHPYFDY

SEQ ID NO: 185 - protein, 5 aa, Artificial Sequence
  CDR1 of clones ADI-29379(E79), 29378(E78) - VH
SYYMH

SEQ ID NO: 186 - protein, 5 aa, Artificial Sequence
  CDR1 of clone ADI-29463(F63) - VH
GYYMH

SEQ ID NO: 187 - protein, 15 aa, Artificial Sequence
  CDR3 of clone ADI-29463(F63) - VH
DTGEYYDTDDHGMDV

SEQ ID NO: 188 - protein, 5 aa, Artificial Sequence
  CDR1 of clone ADI-27744(A44) - VH
SYAMS

SEQ ID NO: 189 - protein, 12 aa, Artificial Sequence
  CDR3 of clone ADI-27744(A44) - VH
DGGYYDSGAGDY

SEQ ID NO: 190 - protein, 5 aa, ARTIFICIAL SEQUENCE
  CDR1 of clones ADI-27744(A44), A49MI, A49ML, A49MV, A49-consensus - VH
SYSMN

SEQ ID NO: 191 - protein, 13 aa, Artificial Sequence
  CDR3 of clone ADI-27744(A44) - VH
GAPMGAAAGWFDP

SEQ ID NO: 192 - protein, 16 aa, Artificial Sequence
  CDR3 of clone 29378(E78) - VH
EGAGFAYGMDYYYMDV

SEQ ID NO: 193 - protein, 13 aa, Artificial Sequence
  CDR3 of clone A49MI - VH
GAPIGAAAGWFDP

SEQ ID NO: 194 - protein, 13 aa, Artificial Sequence
  CDR3 of clone A49MQ - VH
GAPQGAAAGWFDP

SEQ ID NO: 195 - protein, 13 aa, Artificial Sequence
  CDR3 of clone A49ML - VH
GAPLGAAAGWFDP

SEQ ID NO: 196 - protein, 13 aa, Artificial Sequence
  CDR3 of Clone A49MF - VH
GAPFGAAAGWFDP

SEQ ID NO: 197 - protein, 13 aa, Artificial Sequence
  CDR3 of clone A49MV - VH
GAPVGAAAGWFDP

SEQ ID NO: 198 - protein, 5 aa, Artificial Sequence
  linker sequence
  Repeat (1)..(5): amino acids can be repeated 1-10 times
GGGGS

(19) United States
(12) Patent Application Publication
(10) Pub. No.: US 2023/0364230 A1
(43) Pub. Date: Nov. 16, 2023

(54) COMBINATION THERAPY FOR CANCER

(71) Applicant: ENGMAB SÀRL, Boudry (CH)

(72) Inventors: William Pierceall, Madison, NJ (US); Patrick Hagner, Sparta, NJ (US); Hsiling Chiu, Livingston, NJ (US); Safiyyah N. Ziyad, Seattle, WA (US); Anjan Guha Thakurta, Basking Ridge, NJ (US); Mark Orr, Seattle, WA (US)

(21) Appl. No.: 18/044,730

(22) PCT Filed: Sep. 10, 2021
(86) PCT No.: PCT/EP2021/075012

§ 371 Date: Mar. 9, 2023

Related U.S. Application Data
(60) Provisional application No. 63/077,233, filed on Sep. 11, 2020.

(51) Int. Cl. A61K 39/395; A61K 31/5377; A61K 31/517; A61K 31/454; A61P 35/00

(52) U.S. Cl. CPC A61K 39/3955; A61K 31/5377; A61K 31/517; A61K 31/454; A61K 39/39558; A61P 35/00; A61K 2039/505

(57) ABSTRACT

The present application relates to methods of treating, managing and/or preventing cancer, comprising administering (a) a protein that comprises (i) an antigen-binding site that binds an antigen on a natural killer (NK) cell, and (ii) an antigen-binding site that binds an antigen on a cell of said cancer; and (b) iberdomide (CC-220), avadomide (CC-122), lenalidomide, pomalidomide, or CC-92480. The present application also relates to methods of treating, managing and/or preventing cancer, comprising administering (a) a protein that comprises (i) an antigen-binding site that binds NKG2D, and (ii) an antigen-binding site that binds an antigen on a cell of said cancer, and (ii) an antigen-binding site that binds an antigen on a cell of said cancer; and (b) iberdomide (CC-220), avadomide (CC-122), lenalidomide, pomalidomide, or CC-92480.

This application claims priority to U.S. Provisional Application No. 63/077,233, filed on Sep. 11, 2020, the entirety of which is incorporated herein by reference.

### SEQUENCE LISTING

This application incorporates by reference in its entirety the Computer Readable Form (CRF) of a Sequence Listing in ASCII text format. The Sequence Listing text file is entitled “14247-554-228_SeqListing_ST25,” was created on Sep. 10, 2021, and is 136,925 bytes in size.

### 1. INTRODUCTION

The present application relates to methods of treating, managing and/or preventing cancer, comprising administering (a) a protein that comprises (i) an antigen-binding site that binds an antigen on a natural killer (NK) cell, and (ii) an antigen-binding site that binds an antigen on a cell of said cancer; and (b) iberdomide (CC-220), avadomide (CC-122), lenalidomide, pomalidomide, or CC-92480. The present application also relates to methods of treating, managing and/or preventing cancer, comprising administering (a) a protein that comprises (i) an antigen-binding site that binds NKG2D, and (ii) an antigen-binding site that binds an antigen on a cell of said cancer, and (ii) an antigen-binding site that binds an antigen on a cell of said cancer; and (b) iberdomide (CC-220), avadomide (CC-122), lenalidomide, pomalidomide, or CC-92480.

### 2. BACKGROUND

Cancer continues to be a significant health problem despite the substantial research efforts and scientific advances reported in the literature for treating this disease. Some of the most frequently diagnosed cancers include prostate cancer, breast cancer, and lung cancer. Prostate cancer is the most common form of cancer in men. Breast cancer remains a leading cause of death in women. Current treatment options for these cancers are not effective for all patients and/or can have substantial adverse side effects. Other types of cancer also remain challenging to treat using existing therapeutic options.

Cancer immunotherapies are desirable because they are highly specific and can facilitate destruction of cancer cells using the patient's own immune system. Fusion proteins such as bi-specific T-cell engagers are cancer immunotherapies described in the literature that bind to tumor cells and T-cells to facilitate destruction of tumor cells.

Natural killer (NK) cells are a component of the innate immune system and make up approximately 15% of circulating lymphocytes. NK cells infiltrate virtually all tissues and were originally characterized by their ability to kill tumor cells effectively without the need for prior sensitization. Activated NK cells kill target cells by means similar to cytotoxic T cells—i.e., via cytolytic granules that contain perforin and granzymes as well as via death receptor pathways. Activated NK cells also secrete inflammatory cytokines such as IFN-γ and chemokines that promote the recruitment of other leukocytes to the target tissue.

NK cells respond to signals through a variety of activating and inhibitory receptors on their surface. For example, when NK cells encounter healthy self-cells, their activity is inhibited through activation of the killer-cell immunoglobulin-like receptors (KIRs). Alternatively, when NK cells encounter foreign cells or cancer cells, they are activated via their activating receptors (e.g., NKG2D, NCRs, DNAM1). NK cells are also activated by the constant region of some immunoglobulins through CD16 receptors on their surface. The overall sensitivity of NK cells to activation depends on the sum of stimulatory and inhibitory signals.

BCMA is a transmembrane protein belonging to the TNF-receptor superfamily. It specifically binds to the tumor necrosis factor (ligand) superfamily, member 13b (also known as TNFSF13B/TALL-1/BAFF), leading to NF-κB and MAPK8/JNK activation. Its expression is restricted to the B-cell lineage and has been shown to be important for B cell development and autoimmune response. BCMA also binds to various TRAF family members, and thus may transduce signals for cell survival and proliferation. BCMA is implicated in a variety of cancers, such as multiple myeloma, lymphoma and leukemia.

### 3. SUMMARY OF THE INVENTION

Provided herein are methods of treating, managing and/or preventing cancer.

In one aspect, provided herein is a method of treating, managing and/or preventing cancer, comprising administering to a subject in need of such treatment, management and/or prevention a therapeutically or prophylactically effective combination of:
(a) a protein that comprises (i) an antigen-binding site that binds an antigen on a natural killer (NK) cell, and (ii) an antigen-binding site that binds an antigen on a cell of said cancer; and
(b) iberdomide (CC-220), avadomide (CC-122), or lenalidomide.

In another aspect, provided herein is a method of treating, managing and/or preventing cancer, comprising administering to a subject in need of such treatment, management and/or prevention a therapeutically or prophylactically effective combination of:
(a) a protein that comprises (i) an antigen-binding site that binds an antigen on a natural killer (NK) cell, and (ii) an antigen-binding site that binds an antigen on a cell of said cancer; and
(b) iberdomide (CC-220), avadomide (CC-122), lenalidomide, or pomalidomide, and
wherein the method does not comprise administering IL-2.

In another aspect, provided herein is a method of treating, managing and/or preventing cancer, comprising administering to a subject in need of such treatment, management and/or prevention a therapeutically or prophylactically effective combination of:
(a) a protein that comprises (i) an antigen-binding site that binds NKG2D, and (ii) an antigen-binding site that binds an antigen on a cell of said cancer; and
(b) iberdomide (CC-220), avadomide (CC-122), or lenalidomide.

In yet another aspect, provided herein is a method of treating, managing and/or preventing cancer, comprising administering to a subject in need of such treatment, management and/or prevention a therapeutically or prophylactically effective combination of:
(a) a protein that comprises (i) an antigen-binding site that binds NKG2D, and (ii) an antigen-binding site that binds an antigen on a cell of said cancer; and
(b) iberdomide (CC-220), avadomide (CC-122), lenalidomide, or pomalidomide, and wherein the method does not comprise administering IL-2.

In yet another aspect aspect, provided herein is a method of treating, managing and/or preventing cancer, comprising administering to a subject in need of such treatment, management and/or prevention a therapeutically or prophylactically effective combination of:
(a) a protein that comprises (i) an antigen-binding site that binds an antigen on a natural killer (NK) cell, and (ii) an antigen-binding site that binds an antigen on a cell of said cancer; and
(b) CC-92480.

In another aspect, provided herein is a method of treating, managing and/or preventing cancer, comprising administering to a subject in need of such treatment, management and/or prevention a therapeutically or prophylactically effective combination of:
(a) a protein that comprises (i) an antigen-binding site that binds NKG2D, and (ii) an antigen-binding site that binds an antigen on a cell of said cancer; and (b) CC-92480.

In certain embodiments, the method does not comprise administering IL-2.

In certain embodiments, said antigen-binding site that binds to an antigen on an NK cell binds NKG2D.

In certain embodiments, said protein and said iberdomide (CC-220), avadomide (CC-122), lenalidomide, pomalidomide, or CC-92480 activate NK cells to a greater degree than either of said protein or said iberdomide (CC-220), avadomide (CC-122), lenalidomide, pomalidomide, or CC-92480, alone.

In certain embodiments, said antigen on said cancer cell is BCMA.

In certain embodiments, said protein further comprises an antibody Fc domain capable of binding CD16, a portion of an antibody Fc domain capable of binding CD16, or an antigen-binding site that binds CD16.

In certain embodiments, said antigen-binding site that binds NKG2D comprises a single-chain variable fragment (scFv) that binds NKG2D. In certain embodiments, said scFv that binds to NKG2D is linked to said antibody Fc domain capable of binding CD16, said portion of an antibody Fc domain capable of binding CD16, or said antigen-binding site that binds CD16.

In certain embodiments, said antigen-binding site that binds NKG2D comprises a Fab that binds NKG2D. In certain embodiments, said Fab that binds to NKG2D is linked to said antibody Fc domain capable of binding CD16, said portion of an antibody Fc domain capable of binding CD16, or said antigen-binding site that binds CD16.

In certain embodiments, said antigen-binding site that binds NKG2D comprises:
(a) a heavy chain variable domain comprising complementarity-determining region 1 (CDR1) that comprises the amino acid sequence of SEQ ID NO:190, complementarity-determining region 2 (CDR2) that comprises the amino acid sequence of SEQ ID NO:96, and complementarity-determining region 3 (CDR3) that comprised the amino acid sequence of SEQ ID NO:191; and
(b) a light chain variable domain comprising CDR1 that comprises the amino acid sequence of SEQ ID NO:99, CDR2 that comprises the amino acid sequence of SEQ ID NO:100, and CDR3 that comprises the amino acid sequences of SEQ ID NO:101.

In certain embodiments, said antigen-binding site that binds NKG2D comprises:
(a) a heavy chain variable domain comprising the amino acid sequence of SEQ ID NO:190, the amino acid sequence of SEQ ID NO:96, and the amino acid sequence of SEQ ID NO:191; and
(b) a light chain variable domain comprising the amino acid sequence of SEQ ID NO:99, the amino acid sequence of SEQ ID NO:100, and the amino acid sequences of SEQ ID NO:101.

In certain embodiments, said antigen-binding site that binds NKG2D comprises
(a) a heavy chain variable domain at least 90% identical to SEQ ID NO:94 and a light chain variable domain at least 90% identical to SEQ ID NO:98;
(b) a heavy chain variable domain at least 95% identical to SEQ ID NO:94 and a light chain variable domain at least 95% identical to SEQ ID NO:98; or
(c) a heavy chain variable domain identical to SEQ ID NO:94 and a light chain variable domain identical to SEQ ID NO:98.

In certain embodiments, said antigen-binding site that binds BCMA comprises:
(a) a heavy chain variable domain comprising CDR1 that comprises the amino acid sequence of SEQ ID NO:149, CDR2 that comprises the amino acid sequence of SEQ ID NO:150, and CDR3 that comprises the amino acid sequence of SEQ ID NO:151, and
(b) a light chain variable domain comprising CDR1 that comprises the amino acid sequence of SEQ ID NO:153, CDR2 that comprises the amino acid sequence of SEQ ID NO:154, and CDR3 that comprises the amino acid sequence of SEQ ID NO:155.

In certain embodiments, said antigen-binding site that binds BCMA comprises:
(a) a heavy chain variable domain comprising the amino acid sequence of SEQ ID NO:149, the amino acid sequence of SEQ ID NO:150, and the amino acid sequence of SEQ ID NO:151, and
(b) a light chain variable domain comprising the amino acid sequence of SEQ ID NO:153, the amino acid sequence of SEQ ID NO:154, and the amino acid sequence of SEQ ID NO:155.

In certain embodiments, said antigen-binding site that binds BCMA comprises:
(a) a heavy chain variable domain at least 90% identical to SEQ ID NO:148 and a light chain variable domain at least 90% identical to SEQ ID NO:152;
(b) a heavy chain variable domain at least 95% identical to SEQ ID NO:148 and a light chain variable domain at least 95% identical to SEQ ID NO:152; or
(c) a heavy chain variable domain identical to SEQ ID NO:148 and a light chain variable domain identical to SEQ ID NO:152.

In certain embodiments, said protein further comprises an additional antigen-binding site that binds said antigen on a cell of said cancer. In certain embodiments, said additional antigen-binding site binds BCMA.

In certain embodiments, said additional antigen-binding site that binds BCMA comprises:
(a) a heavy chain variable domain comprising CDR1 that comprises the amino acid sequence of SEQ ID NOs:149, CDR2 that comprises the amino acid sequence of SEQ ID NO:150, and CDR3 that comprises the amino acid sequence of SEQ ID NOs:151, and
(b) a light chain variable domain comprising CDR1 that comprises the amino acid sequence of SEQ ID NOs:153, CDR2 that comprises the amino acid sequence of SEQ ID NO:154, and CDR3 that comprises the amino acid sequence of SEQ ID NOs:155.

In certain embodiments, said additional antigen-binding site that binds BCMA comprises:
(a) a heavy chain variable domain at least 90% identical to SEQ ID NO:148 and a light chain variable domain at least 90% identical to SEQ ID NO:152;
(b) a heavy chain variable domain at least 95% identical to SEQ ID NO:148 and a light chain variable domain at least 95% identical to SEQ ID NO:152; or
(c) a heavy chain variable domain identical to SEQ ID NO:148 and a light chain variable domain identical to SEQ ID NO:152.

In certain embodiments, said protein comprises an antibody Fc domain.

In certain embodiments, said antibody Fc domain comprises hinge and CH2 domains of a human IgG1 antibody.

In certain embodiments, said Fc domain comprises an amino acid sequence at least 90% identical to amino acids 234-332 of a human IgG1 antibody.

In certain embodiments, said protein comprises a polypeptide comprising the amino acid sequence of SEQ ID NO:161.

In certain embodiments, said protein comprises a polypeptide comprising an amino acid sequence at least 90%, at least 95% or at least 99% identical to the amino acid sequence of SEQ ID NO:162. In certain embodiments, said protein comprises (i) a polypeptide comprising the amino acid sequence of SEQ ID NO:162. In certain embodiments, said protein further comprises (ii) a polypeptide comprising the amino acid sequence of SEQ ID NO:163; and (iii) a polypeptide comprising the amino acid sequence of SEQ ID NO:165. In certain embodiments, said protein further comprises (ii) a polypeptide comprising the amino acid sequence of SEQ ID NO:163; and (iii) two polypeptides each comprising the amino acid sequence of SEQ ID NO:165.

In certain embodiments, said polypeptides are linked as represented in FIG. 15.

In certain embodiments, said polypeptide comprising the amino acid sequence of SEQ ID NO:162 is linked to the polypeptide comprising the amino acid sequence of SEQ ID NO:163 via heterodimerization and at least one disulfide bond; wherein the polypeptide comprising the amino acid sequence of SEQ ID NO:165 is linked to the polypeptide comprising the amino acid sequence of SEQ ID NO:162 via a disulfide bond; and wherein the other polypeptide comprising the amino acid sequence of SEQ ID NO:165 is linked to the polypeptide comprising the amino acid sequence of SEQ ID NO:163 via a disulfide bond.

In another aspect, provided herein is a method of treating, managing and/or preventing cancer, comprising administering to a subject in need of such treatment, management and/or prevention a therapeutically or prophylactically effective combination of:
(a) a protein that comprises (i) a polypeptide comprising the amino acid sequence of SEQ ID NO:162; (ii) a polypeptide comprising the amino acid sequence of SEQ ID NO:163; and (iii) two polypeptides each comprising the amino acid sequence of SEQ ID NO:165, and
(b) iberdomide (CC-220), avadomide (CC-122), lenalidomide, or pomalidomide,
wherein the polypeptide comprising the amino acid sequence of SEQ ID NO:162 is linked to the polypeptide comprising the amino acid sequence of SEQ ID NO:163 via heterodimerization and at least one disulfide bond; wherein the polypeptide comprising the amino acid sequence of SEQ ID NO:165 is linked to the polypeptide comprising the amino acid sequence of SEQ ID NO:162 via a disulfide bond; and wherein the other polypeptide comprising the amino acid sequence of SEQ ID NO:165 is linked to the polypeptide comprising the amino acid sequence of SEQ ID NO:163 via a disulfide bond.

In another aspect, provided herein is a method of treating, managing and/or preventing cancer, comprising administering to a subject in need of such treatment, management and/or prevention a therapeutically or prophylactically effective combination of:
(a) a protein that comprises (i) a polypeptide comprising the amino acid sequence of SEQ ID NO:162; (ii) a polypeptide comprising the amino acid sequence of SEQ ID NO:163; and (iii) two polypeptides each comprising the amino acid sequence of SEQ ID NO:165, and
(b) CC-92480,
wherein the polypeptide comprising the amino acid sequence of SEQ ID NO:162 is linked to the polypeptide comprising the amino acid sequence of SEQ ID NO:163 via heterodimerization and at least one disulfide bond; wherein the polypeptide comprising the amino acid sequence of SEQ ID NO:165 is linked to the polypeptide comprising the amino acid sequence of SEQ ID NO:162 via a disulfide bond; and wherein the other polypeptide comprising the amino acid sequence of SEQ ID NO:165 is linked to the polypeptide comprising the amino acid sequence of SEQ ID NO:163 via a disulfide bond.

In certain embodiments, said cancer is leukemia, myeloma, non-Hodgkin's lymphoma (NHL), Hodgkin's lymphoma (HL), and Waldenstrom's macroglobulinemia.

In certain embodiments, said cancer is acute lymphoblastic leukemia (ALL), acute myeloid leukemia (AML), chronic lymphocytic leukemia/small lymphocytic lymphoma (CLL/SLL), chronic myeloid leukemia (CML), chronic myelomonocytic leukemia (CMML), multiple myeloma (MM), classic Hodgkin lymphoma (cHL), nodular lymphocyte-predominant Hodgkin lymphoma, follicular lymphoma (FL), lymphoplasmacytic lymphoma, marginal zone lymphoma (MZL), primary cutaneous anaplastic large cell lymphoma, diffuse large B-cell lymphoma (DLBCL), follicular large cell lymphoma, anaplastic large cell lymphoma, extranodal NK-/T-cell lymphoma, lymphomatoid granulomatosis, angioimmunoblastic T-cell lymphoma, peripheral T-cell lymphoma (PTCL), primary central nervous system lymphoma (PCNSL), intravascular large B-cell lymphoma, mantle cell lymphoma (MCL), post-transplantation lymphoproliferative disorder, Burkitt lymphoma, lymphoblastic lymphoma, adult T-cell leukemia/lymphoma, B-cell lymphomas, glioblastoma, true histiocytic lymphoma, primary effusion lymphoma, or plasmablastic lymphoma.

In certain embodiments, said cancer is relapsed or refractory. In certain embodiments, said cancer is newly diagnosed. In certain embodiments, said subject has failed at least one prior therapy.

In certain embodiments, said subject is less than 18 years old. In certain embodiments, said subject is 18 years old or older.

In certain embodiments, the combination of (a) said protein and (b) said iberdomide (CC-220), avadomide (CC-122), lenalidomide, or pomalidomide is in a therapeutically effective amount. In certain embodiments, said protein is in a therapeutically effective amount. In certain embodiments, said iberdomide (CC-220), avadomide (CC-122), lenalidomide, or pomalidomide is in a therapeutically effective amount.

In certain embodiments, said method comprises administering iberdomide (CC-220). In certain embodiments, said iberdomide is administered in an amount of about 0.1 mg to about 2 mg per day.

In certain embodiments, said method comprises administering avadomide (CC-122). In certain embodiments, said avadomide is administered in an amount of about 2 mg to about 50 mg per day.

In certain embodiments, said method comprises administering lenalidomide. In certain embodiments, said lenalidomide is administered in an amount of about 10 mg to about 25 mg per day.

In certain embodiments, said method comprises administering pomalidomide. In certain embodiments, said pomalidomide is administered in an amount of about 3 mg to about 5 mg per day.

In certain embodiments, said method comprises administering CC-92480. In certain embodiments, said CC-92480 is administered in an amount of about 0.1 mg to about 2 mg per day.

In certain embodiments, said iberdomide, avadomide, lenalidomide, pomalidomide, or CC-92480 is administered on days 1 to 5 of a 7-day cycle or days 1 to 21 of a 28-day cycle.

In certain embodiments, said method further comprises administering a third agent that is or comprises dexamethasone, iberdomide, avadomide, lenalidomide, pomalidomide, or CC-92480.

### 3.1 Definitions

As used herein and unless otherwise indicated, the term “treating” means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself.

As used herein and unless otherwise indicated, the term “preventing” means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition.

As used herein and unless otherwise indicated, the term “managing” encompasses preventing the recurrence of the particular disease or disorder in a patient who had suffered from it, lengthening the time a patient who had suffered from the disease or disorder remains in remission, reducing mortality rates of the patients, and/or maintaining a reduction in severity or avoidance of a symptom associated with the disease or condition being managed.

As used herein and unless otherwise indicated, the term “effective amount” in connection with an agent (e.g., a protein described herein) means an amount that treats, prevents, or manages a disorder, disease or condition, or symptoms thereof, or is capable of treating, preventing, or managing a disorder, disease or condition, or symptoms thereof.

As used herein and unless otherwise indicated, the term “subject” or “patient” includes an organism, including, but not limited to, an animal such a cow, monkey, horse, sheep, pig, chicken, turkey, quail, cat, dog, mouse, rat, rabbit or guinea pig. In one embodiment, the subject or patient is a mammal. In certain embodiments, the subject or patient is a non-human primate. In another embodiment, the subject or patient is a human.

As used herein and unless otherwise indicated, the term “relapsed” refers to a disorder, disease, or condition that at one time had responded to treatment (e.g., achieved a complete response) then had progression. The treatment can include one or more lines of therapy. In one embodiment, the disorder, disease or condition has been previously treated with one or more lines of therapy. In another embodiment, the disorder, disease or condition had been previously treated with one, two, three, four, or more lines of therapy.

As used herein and unless otherwise indicated, the term “refractory” refers to a disorder, disease, or condition that has not responded, or has responded unsatisfactorily, to prior treatment; such prior treatment can include one or more lines of therapy. In one embodiment, the refractory disorder, disease, or condition has been previously treated with one, two, three or four lines of therapy. In one embodiment, the disorder, disease, or condition has been previously treated with two or more lines of treatment, and has less than a complete response (CR) to most recent systemic therapy containing regimen.

In one embodiment, “relapsed or refractory” cancer may refer to cancer that has been previously treated with one or more lines of therapy. In one embodiment, the relapsed or refractory cancer is cancer that has been previously treated with one, two, three or four lines of therapy. In one embodiment, the relapsed or refractory cancer is cancer that has been previously treated with two or more lines of therapy.

Combination therapy or “in combination with” refer to the use of more than one therapeutic agent to treat a particular disease, disorder or condition, e.g., a combination of a protein as described herein with one or more of lenalidomide, pomalidomide, avadomide, iberdomide, or CC-92480 to treat a particular disease, disorder or condition. “In combination with” or “combination therapy” is not intended to imply that the therapeutic agents must be administered at the same time and/or formulated for delivery together, although these methods of delivery are within the scope of this disclosure. A therapeutic agent can be administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), one or more other additional agents. The therapeutic agents in a combination therapy can also be administered on an alternating dosing schedule, with or without a resting period (e.g., no therapeutic agent is administered on certain days of the schedule). The administration of a therapeutic agent “in combination with” another therapeutic agent includes, but is not limited to, sequential administration and concomitant administration of the two agents. In general, each therapeutic agent is administered at a dose and/or on a time schedule determined for that particular agent.

As used herein, the term “pharmaceutically acceptable salt” refers to any pharmaceutically acceptable salt (e.g., acid or base) of a compound which, upon administration to a subject, is capable of providing a compound or an active metabolite or residue thereof.

As is known to those of skill in the art, “salts” of a compound may be derived from inorganic or organic acids and bases. Exemplary acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts. Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N dibenzylethylenediamine, chloroprocaine, choline, diethanolamine, ethylenediamine, meglumaine (N-methylglucamine), lysine, and procaine.

As used herein, and unless otherwise specified, the term “about” or “approximately” means an acceptable error for a particular value as determined by one of ordinary skill in the art, which depends in part on how the value is measured or determined. In certain embodiments, the term “about” or “approximately” means within 1 or 2 standard deviations. In certain embodiments, the term “about” or “approximately” means within 10%, 9%, 8%, 7%, 6%, 5%, 4%, 3%, 2%, 1%, 0.5%, or 0.05% of a given value or range.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA [1975].

### 3.2 Cancer-Related Clinical Trial Endpoints

“Overall survival” (OS) is defined as the time from first dose until death from any cause, and is measured in the intent-to-treat population. Overall survival can be evaluated in randomized controlled studies. Demonstration of a statistically significant improvement in overall survival can be considered to be clinically significant if the toxicity profile is acceptable, and has often supported new drug approval.

Several endpoints are based on cancer assessments. These endpoints include disease-free survival (DFS), objective response rate, time to progression (TTP), progression-free survival (PFS), event-free survival (EFS), duration of response (DOR) and time-to-treatment failure (TTF). The collection and analysis of data on these time-dependent endpoints are based on indirect assessments, calculations, and estimates.

Generally, “disease-free survival” (DFS) is defined as the time from randomization until recurrence of cancer or death from any cause. Although overall survival is a conventional endpoint for most adjuvant settings, DFS can be an important endpoint in situations where survival may be prolonged, making a survival endpoint impractical. DFS can be a surrogate for clinical benefit or it can provide direct evidence of clinical benefit. This determination is based on the magnitude of the effect, its risk-benefit relationship, and the disease setting. The definition of DFS can be complicated, particularly when deaths are noted without prior cancer progression documentation. These events can be scored either as disease recurrences or as censored events. Although all methods for statistical analysis of deaths have some limitations, considering all deaths (deaths from all causes) as recurrences can minimize bias. DFS can be overestimated using this definition, especially in patients who die after a long period without observation. Bias can be introduced if the frequency of long-term follow-up visits is dissimilar between the study arms or if dropouts are not random because of toxicity.

“Objective response rate” is defined as the sum of the percentage of patients who achieve complete and partial responses. Response duration usually is measured from the time of initial response until documented cancer progression. Generally, the FDA has defined objective response rate as the sum of partial responses plus complete responses. When defined in this manner, objective response rate is a direct measure of drug anticancer activity, which can be evaluated in a single-arm study. If available, standardized criteria should be used to ascertain response. A variety of response criteria have been considered appropriate (e.g., RECIST criteria) (Therasse et al, J Natl Cancer Inst, 92:205-16 (2000)). The significance of objective response rate is assessed by its magnitude and duration, and the percentage of complete responses (no detectable evidence of cancer).

“Duration of response” (DOR) is the time from achieving a response until relapse or disease progression.

“Time to progression” (TTP) and “progression-free survival” (PFS) have served as primary endpoints for drug approval. TTP is defined as the time from randomization until objective cancer progression; TTP does not include deaths. PFS is defined as the time from randomization until objective cancer progression or death. Compared with TTP, PFS is the preferred regulatory endpoint. PFS includes deaths and thus can be a better correlate to overall survival. PFS assumes patient deaths are randomly related to cancer progression. However, in situations where the majority of deaths are unrelated to cancer, TTP can be an acceptable endpoint.

As an endpoint to support drug approval, PFS can reflect cancer growth and be assessed before the determination of a survival benefit. Its determination is not confounded by subsequent therapy. For a given sample size, the magnitude of effect on PFS can be larger than the effect on overall survival. However, the formal validation of PFS as a surrogate for survival for the many different malignancies that exist can be difficult. Data are sometimes insufficient to allow a robust evaluation of the correlation between effects on survival and PFS. Cancer trials are often small, and proven survival benefits of existing drugs are generally modest. The role of PFS as an endpoint to support licensing approval varies in different cancer settings. Whether an improvement in PFS represents a direct clinical benefit or a surrogate for clinical benefit depends on the magnitude of the effect and the risk-benefit of the new treatment compared to available therapies.

“Event-free survival” (EFS) is the time from study entry until any treatment failure, including disease progression, treatment discontinuation for any reason, or death.

“Time-to-treatment failure” (TTF) is defined as a composite endpoint measuring time from randomization to discontinuation of treatment for any reason, including disease progression, treatment toxicity, and death. TTF is not recommended as a regulatory endpoint for drug approval. TTF does not adequately distinguish efficacy from these additional variables. A regulatory endpoint should clearly distinguish the efficacy of the drug from toxicity, patient or physician withdrawal, or patient intolerance.

In certain embodiments, the methods provided herein are useful for achieving one or more of these clinical trial endpoints in a patient. In certain embodiments, the methods provided herein are useful for improving one or more of these clinical trial endpoints in a patient.

### 3.3 Conventions and Abbreviations

| Abbreviation | Convention |
|---|---|
| ADCC | antibody-dependent cellular cytotoxicity |
| AE | adverse event |
| AML | acute myeloid leukemia |
| BCMA | B cell maturation antigen |
| CDR | complementarity-determining region |
| cHL | classic Hodgkin lymphoma |
| ChIP-seq | chromatin immunoprecipitation sequencing |
| CLL/SLL | chronic lymphocytic leukemia/small lymphocytic |
| | lymphoma |
| CML | chronic myeloid leukemia |
| CMML | chronic myelomonocytic leukemia |
| CRR | complete response rate |
| DFS | disease-free survival |
| DLBCL | diffuse large B-cell lymphoma |
| DLT | dose-limiting toxicity |
| DOR | duration of response |
| EFS | event-free survival |
| Fab | Antigen binding fragment of antibody |
| Fc | Fragment crystallizable region |
| FcγRIIIa(F) | Fc gamma receptor IIIa F allele, also known as CD16 (F) |
| FcγRIIIa(V) | Fc gamma receptor IIIa V allele, also known as CD16 (V) |
| HL | Hodgkin's lymphoma |
| Human IgG | Human immunoglobulin G |
| IgG1 | Immunoglobulin G1 |
| ITT | intent-to-treat |
| mAb | monoclonal antibody |
| MAD | maximum administered dose |
| MM | multiple myeloma |
| MTD | maximum tolerated dose |
| MZL | marginal zone lymphoma |
| NHL | non-Hodgkin's lymphoma |
| NK cells | natural killer cells |
| NKG2D | natural killer group 2, member D receptor |
| OS | overall survival |
| PBMC | peripheral blood mononuclear cell |
| PFS | progression-free survival |
| scFv | single-chain Fragment variable |
| sCR | strict complete response |
| TriNKET | tri-specific NK cell engager therapy |
| TTF | time-to-treatment failure |
| TTP | time to progression |
| TTR | time to response |
| VGPR | very good partial response or better rate |

### 4. BRIEF DESCRIPTION OF THE FIGURES

FIGS. 1A-1B illustrate exemplary trispecific antibodies (TriNKET), which include an scFv antigen-binding site that binds NKG2D, an antigen-binding site that binds BCMA, an additional tumor-associated antigen-binding site that binds BCMA, and a heterodimerized antibody constant region that binds CD16. These antibody formats are referred herein as F4-TriNKET. FIG. 1A illustrates that the two BCMA-binding sites in the Fab format. FIG. 1B illustrates that the two BCMA-binding sites in the scFv format.

FIG. 2 illustrates an exemplary TriNKET that contains an scFv antigen-binding site that binds NKG2D or BCMA, an antigen-binding site that binds BCMA or NKG2D, and a heterodimerized antibody constant region that binds CD16. The antibody format is referred herein as F3-TriNKET.

FIGS. 3A-3B illustrate degradation of Aiolos (FIG. 3A) and Ikaros (FIG. 3B) in primary NK cells mediated by iberdomide, avadomide, and lenalidomide. “Conc.” means concentration.

FIG. 4 illustrates enhanced NK cell proliferation in DLBCL patient samples by avadomide and lenalidomide in a dose dependent manner. “Len” means lenalidomide.

FIG. 5 illustrates significantly expanded NKG2D+ NK cells in DLBCL patient PBMC by avadomide and lenalidomide.

FIGS. 6A-6B illustrate enhanced NK cell mediated cytotoxicity and antibody-dependent cellular cytotoxicity (ADCC) against CLL cell lines by iberdomide. FIG. 6A: MEC2 cells; FIG. 6B: WA-OSEL cells.

FIG. 7A illustrates degradation of Aiolos and Ikaros and correlated induction of interferon stimulated genes mediated by avadomide in DLBCL cells. “Ava” means avadomide.

FIG. 7B illustrates pathway analysis of proteins induced in CLL cells treated with avadomide.

FIG. 8 illustrates that Aiolos and Ikaros ChIP-seq revealed binding to MICA promotor.

FIGS. 9A-9B illustrate that Aiolos and Ikaros directly regulate promoters of MEW I/II and immune recognition genes in DLBCL cell lines.

FIG. 10A illustrates increased expression of HLA-DR and CD86 in TMD8 cells by avadomide (CC-122) treatment.

FIG. 10B illustrates ChIP-seq signals of Aiolos and Ikaros on DLBCL cells.

FIGS. 11A-11B illustrate that pretreatment of PBMCs with different concentrations of CC-92480 enhanced Protein 1 induced ADCC. Graphs represent data from one of two different healthy PBMC donors that were pretreated with 0 nM to 10 nM of CC-92480 for either 24 hours (FIG. 11A) or 48 hours (FIG. 11B). After pretreatment, PBMCs were cultured with H929 target cells with or without 0.1 nM Protein 1 overnight. H929 lysis was determined via flow cytometry.

FIGS. 12A-12B illustrate that pretreatment of PBMCs with different concentrations of CC-220 enhanced Protein 1 induced ADCC. Graphs represent data from one of two different healthy PBMC donors that were pretreated with 0 nM to 100 nM of CC-220 for either 24 (FIG. 12A) or 48 hours (FIG. 12B). After pretreatment, PBMCs were cultured with H929 target cells with or without 0.1 nM Protein 1 overnight. H929 lysis was determined via flow cytometry.

FIGS. 13A-13B illustrate that pretreatment of PBMCs with single concentration of CC-92480 or CC-220 enhanced Protein 1 induced ADCC. PBMCs were pretreated 0.1 nM of CC-92480 or 1 nM of CC-220 or DMSO control for either 24 hours (FIG. 13A) or 48 hours (FIG. 13B). After pretreatment, PBMC were cultured with H929 target cells and 0 nM to 10 nM of Protein 1 overnight. H929 lysis was determined via flow cytometry.

FIGS. 14A-14B illustrate that pretreatment of both PBMC and H929 target cells with CC-92480 or CC-220 enhanced Protein 1 induced ADCC. PBMCs and H929 cells were pretreated separately with varying doses of CC-92480 (FIG. 14A) or CC-220 (FIG. 14B) or a DMSO control for 48 hours. After pretreatment, PBMCs and H929 target cells were combined and co-cultured overnight with a dose range of Protein 1 0 nM to 10 nM. H929 cell depletion was analyzed by flow cytometry.

### 5. DETAILED DESCRIPTION OF THE INVENTION

Provided herein are combination treatments for cancer comprising administering to a subject (a) a protein that comprises (i) an antigen-binding site that binds an antigen on a natural killer (NK) cell, and (ii) an antigen-binding site that binds an antigen on a cell of said cancer, and (b) iberdomide (CC-220), avadomide (CC-122), lenalidomide, pomalidomide, or CC-92480. Also provided herein are combination treatments for cancer comprising administering to a subject (a) a protein that comprises (i) an antigen-binding site that binds NKG2D, and (ii) an antigen-binding site that binds an antigen on a cell of said cancer, and (ii) an antigen-binding site that binds an antigen on a cell of said cancer; and (b) iberdomide (CC-220), avadomide (CC-122), lenalidomide, pomalidomide, or CC-92480. In certain embodiments, the method does not comprise administering IL-2. The protein that comprises an antigen-binding site that binds an antigen on a natural killer (NK) cell is described in Section 5.1. The protein that comprises an antigen-binding site that binds NKG2D is described in Section 5.2. Iberdomide (CC-220) is described in Section 5.3. Avadomide (CC-122) is described in Section 5.4. Lenalidomide is described in Section 5.5. Pomalidomide is described in Section 5.6. CC-92480 is described in Section 5.7.

Methods of the combination treatments are described in Section 5.8. Formulations that can be used are described in Section 5.9. Assays that can be used to demonstrate the efficacy and/or characteristics of the combination treatments are described in Section 5.10, for example the synergistic anti-tumor effects.

### 5.1 Protein that Comprises an Antigen-Binding Site that Binds to an Antigen on a Natural Killer (NK) Cell

Described herein is a protein that comprises an antigen-binding site that binds to an antigen on a natural killer (NK) cell. In certain embodiments, the protein described herein further comprises an antigen-binding site that binds an antigen on a cancer cell. The protein described herein can be administered in combination with iberdomide (CC-220), avadomide (CC-122), lenalidomide, pomalidomide, or CC-92480 as described in Sections 5.3 to 5.7, respectively, in the methods of the combination treatments as described in Section 5.8.

In certain embodiments, the antigen on an NK cell is a receptor expressed on the NK cell. In certain embodiments, the receptor is an activating receptor. Exemplary activating receptors include, but are not limited to, Natural Killer Group 2D (NKG2D), DNAX accessory molecule-1 (DNAM-1), SLAM family receptor 2B4, natural cytotoxicity receptors (NCRs, for example, NKp30, NKp44 and NKp46), activating killer immunoglobulin receptors (KIRs), NKG2C-CD94 and CD16. In certain embodiments, the antigen on an NK cell is NKG2D. In certain embodiments, the antigen on an NK cell is an activating receptor as detailed above.

In certain embodiments, the antigen is specifically or predominantly expressed on an NK cell. In certain embodiments, the antigen on an NK cell is also expressed on an immune cell that is not a NK cell. In certain embodiments, the antigen on an NK cell is also expressed on a T cell. For example, the antigen on an NK cell is NKG2D, which is also expressed on CD8+ T cells, γδ T cells and CD4⁺ T cells.

In certain embodiments, the protein described herein further comprises an antigen-binding site that binds an antigen on a cancer cell. In certain embodiments, said antigen is BCMA.

In certain embodiments, the protein described herein further comprises an antigen-binding site that binds a second antigen on a natural killer (NK) cell. In certain embodiments, the second antigen on a NK cell is a receptor expressed on the NK cell. In certain embodiments, the receptor is an activating receptor. Exemplary activating receptors include, but are not limited to, Natural Killer Group 2D (NKG2D), DNAX accessory molecule-1 (DNAM-1), the SLAM family receptor 2B4, the natural cytotoxicity receptors (NCRs, for example, NKp30, NKp44 and NKp46), activating killer immunoglobulin receptors (KIRs), NKG2C-CD94 and CD16. In certain embodiments, the antigen-binding site that binds the second antigen on an NK cell binds CD16.

In certain embodiments, the protein further comprises an antibody Fc domain capable of binding CD16 or a portion of an antibody Fc domain capable of binding CD16. In certain embodiments, CD16 is expressed on an NK cell. In other embodiments, CD16 is expressed on a cell that is other than an NK cell. Exemplary cells that express CD16 include, but are not limited to, NK cells, cytotoxic T cells, macrophages, neutrophils, eosinophils, mast cells, monocytes and dendritic cells.

Without being bound by any particular theory, the protein may bind antigens on various cells as described in the embodiments below. In certain embodiments, the protein binds an activating receptor on an NK cell. In certain embodiments, the protein binds a receptor on an immune cell that is not an NK cell. In certain embodiments, the protein binds a receptor and CD16 expressed on the same cell, such as an NK cell. In certain embodiments, the protein binds NKG2D and CD16 expressed on the same cell, such as an NK cell. In certain embodiments, the protein binds both BCMA expressed on a cancer cell and a receptor on an NK cell. In certain embodiments, the protein binds both BCMA expressed on a cancer cell and a CD16-expressing cell. In certain embodiments, the protein binds BCMA expressed on a cancer cell and a receptor and CD16 both expressed on the same cell.

Without being bound by any particular theory, the protein may function as described in the embodiments below. In certain embodiments, the protein engages an NK cell or an immune cell expressing the antigen that is also expressed on an NK cell. In certain embodiments, the NK cell or the immune cell mediates the killing of the BCMA-expressing cancer cell. In certain embodiments, the protein also engages a CD16-expressing cell, which is the same or different from the NK cell or the immune cell mentioned above. In certain embodiments, the CD16-expressing cell also mediates the killing of the BCMA-expressing cancer cell. In certain embodiments, the protein facilitates this killing process via binding to the NK cell or the immune cell and the BCMA-expressing cancer cell. In certain embodiments, the protein facilitates this killing process via binding to both the CD16-expressing cell and the BCMA-expressing cancer cell. In certain embodiments, the protein facilitates this killing process via binding to a receptor on an NK cell and CD16 that is expressed on the same NK cell and the BCMA-expressing cancer cell.

In certain embodiments, the protein in combination with iberdomide (CC-220), avadomide (CC-122), lenalidomide, pomalidomide, or CC-92480 activates NK cells to a greater degree than either of the protein, or iberdomide (CC-220), avadomide (CC-122), lenalidomide, pomalidomide, or CC-92480, alone. In certain embodiments, the protein as described herein in combination with iberdomide (CC-220), avadomide (CC-122), lenalidomide, pomalidomide, or CC-92480 activate an immune cell other than NK cells (e.g., NKT cells, CD8+ T cells, γδ T cells, and CD4⁺ T cells) to a greater degree than either of the protein, or iberdomide (CC-220), avadomide (CC-122), lenalidomide, pomalidomide, or CC-92480 alone. In certain embodiments, the protein as described herein has synergistic anti-cancer effect with iberdomide (CC-220), avadomide (CC-122), lenalidomide, pomalidomide, or CC-92480.

In certain embodiments, the protein described herein is a multi-specific binding protein that is described in Section 5.2.1.

### 5.2 Protein that Comprises an Antigen-Binding Site that Binds NKG2D

Described herein is a protein that comprises an antigen-binding site that binds NKG2D. In certain embodiments, the protein described herein further comprises an antigen-binding site that binds an antigen on a cancer cell. The protein described herein can be administered in combination with iberdomide (CC-220), avadomide (CC-122), lenalidomide, pomalidomide, or CC-92480, e.g., in the methods of the combination treatments as described in Section 5.8.

In certain embodiments, the antigen-binding site binds NKG2D on an NK cell. In certain embodiments, the antigen-binding site binds NKG2D on an immune cell that is not an NK cell. In other embodiments, the antigen-binding site binds NKG2D on more than one types of immune cells, for example, two types of immune cells. Immune cells that express NKG2D include, but are not limited to, NK cells, NKT cells, CD8+ T cells, γδ T cells, and CD4⁺ T cells.

In certain embodiments, the protein described herein further comprises an antigen-binding site that binds an antigen on a cancer cell. In certain embodiments, said antigen is B cell maturation antigen (BCMA).

In certain embodiments, the protein described herein further comprises an antigen-binding site that binds a second antigen on a natural killer (NK) cell. In certain embodiments, the second antigen on a NK cell is a receptor expressed on the NK cell. In certain embodiments, the receptor is an activating receptor. Exemplary activating receptors include, but are not limited to, Natural Killer Group 2D (NKG2D), DNAX accessory molecule-1 (DNAM-1), the SLAM family receptor 2B4, the natural cytotoxicity receptors (NCRs, for example, NKp30, NKp44 and NKp46), activating killer immunoglobulin receptors (KIRs), NKG2C-CD94 and CD16. In certain embodiments, the antigen-binding site that binds to the second antigen on an NK cell binds CD16.

In certain embodiments, the protein further comprises an antibody Fc domain that binds CD16, a portion thereof sufficient to bind CD16. In certain embodiments, CD16 is expressed on an NK cell. In other embodiments, CD16 is expressed on a cell that is other than an NK cell. Exemplary cells that express CD16 include, but are not limited to, NK cells, cytotoxic T cells, macrophages, neutrophils, eosinophils, mast cells, monocytes and dendritic cells.

Without being bound by any particular theory, the protein may bind to antigens on various cells as described in the embodiments below. In certain embodiments, the protein binds NKG2D and CD16 expressed on the same cell, such as an NK cell or a T cell. In certain embodiments, the protein binds both BCMA expressed on a cancer cell and an NKG2D-expressing cell. In certain embodiments, the protein binds both BCMA expressed on a cancer cell and a CD16-expressing cell. In certain embodiments, the protein binds BCMA expressed on a cancer cell and NKG2D and CD16 both expressed on the same cell. In certain embodiments, the binding characteristics of the protein is shown in Example 2 (pages 71-73) of PCT/US2019/045632 (PCT/US2019/045632 is incorporated by reference in its entirety).

Without being bound by any particular theory, the protein may function as described in the embodiments below. In certain embodiments, the NKG2D-expressing cell and/or the CD16-expressing cell mediates the killing of a BCMA-expressing cancer cell. In certain embodiments, the protein facilitates this killing process via binding to both the NKG2D-expressing cell and the BCMA-expressing cancer cell. In certain embodiments, the protein facilitates this killing process via binding to both the CD16-expressing cell and the BCMA-expressing cancer cell. In certain embodiments, a cell expressing both NKG2D and CD16, for example an NK cell or a T cell, mediates the killing of the BCMA-expressing cancer cell. In certain embodiments, the protein facilitates this killing process via binding to both the cell expressing both NKG2D and CD16 and the BCMA-expressing cancer cell.

### 5.2.1 Multi-Specific Binding Proteins

(i) Components of the Multi-Specific Binding Proteins

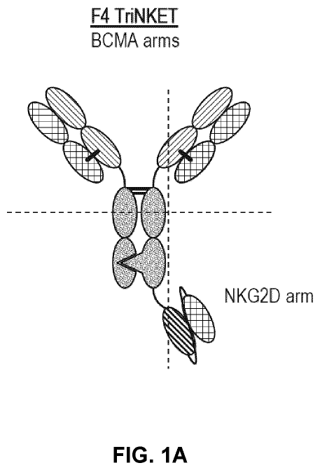

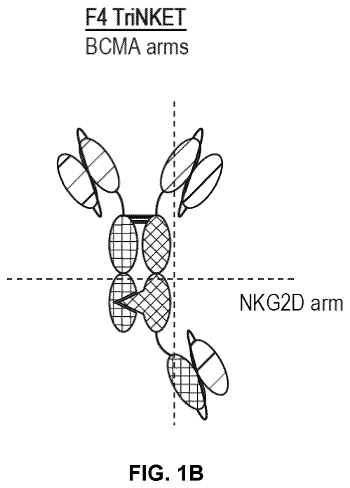

In certain embodiments, the multi-specific binding proteins described herein can take various formats. FIG. 1A illustrates F4 TriNKET having two antigen-binding sites that bind BCMA, wherein both antigen binding sites that bind BCMA are Fab fragment. The F4 TriNKET (Fab) include an first antigen-binding site that binds NKG2D, which comprises a scFv, a second antigen-binding site that binds BCMA, an additional antigen-binding site that binds BCMA, and a heterodimerized antibody constant region that binds CD16. The F4 TriNKET (Fab) is a heterodimeric, multi-specific antibody that includes four peptides: a first immunoglobulin heavy chain, a second immunoglobulin heavy chain and two immunoglobulin light chains (FIG. 1A). The first immunoglobulin heavy chain includes, from N-terminus to C-terminus, a heavy chain variable domain (VH) linked to a heavy chain constant region 1 (CH1) which forms a first (VH-CH1) domain, and a first Fc (hinge-CH2-CH3) domain, wherein the first (VH-CH1) domain pairs with the first light chain to form a first Fab that binds BCMA, and wherein the (VH-CH1) domain is linked to the first Fc via either a linker or a hinge (FIG. 1A). The second immunoglobulin heavy chain includes, from N-terminus to C-terminus, a second (VH-CH1) domain, a second Fc (hinge-CH2-CH3) domain, and a single-chain variable fragment (scFv) that is composed of a VH and a VL that pair and bind NKG2D, wherein the second Fc domain is linked via either a linker or a hinge at its N-terminus to the second (VH-CH1) domain, and via either a linker or a hinge at its C-terminus to the scFv that binds NKG2D, and wherein the second (VH-CH1) domain pairs with the second light chain to form a second Fab that binds BCMA (FIG. 1A). In certain embodiments, the binding characteristics of the multi-specific binding protein is shown in Example 2 (pages 71-73) of PCT/US2019/045632 (PCT/US2019/045632 is incorporated by reference in its entirety).

The F4 TriNKET (scFv) is a heterodimeric, multi-specific antibody that includes two peptides: a first immunoglobulin heavy chain and a second immunoglobulin heavy chain (FIG. 1B). The first immunoglobulin heavy chain includes, from N-terminus to C-terminus, a first scFv that binds BCMA and a first Fc (hinge-CH2-CH3) domain, wherein the first scFv that binds BCMA is linked to the first Fc via either a linker or a hinge (FIG. 1B). The second immunoglobulin heavy chain includes, from N-terminus to C-terminus, a second scFv that binds BCMA, a second Fc (hinge-CH2-CH3) domain, and a scFv that binds NKG2D, wherein the second Fc domain is linked via either a linker or a hinge at its N-terminus to the second scFv domain that binds BCMA, and via either a linker or a hinge at its C-terminus to the scFv that binds NKG2D (FIG. 1B).

TriNKETs termed “NKG2D-binding-F4-TriNKET-BCMA” can refer to the TriNKETs depicted in FIG. 1A (NKG2D-binding-F4 (Fab)-TriNKET-BCMA) or FIG. 1B (NKG2D-binding-F4 (scFv)-TriNKET-BCMA). For example, the TriNKET “A49-F4-TRINKET-BCMA” refers to a TriNKET that has the “NKG2D-binding-F4-TriNKET-BCMA” format, and has an NKG-2D binding domain comprising the VH and VL of A49 (See Table 1 below).

In some embodiments, the single-chain variable fragment (scFv) described above is linked to the antibody constant domain via a hinge sequence. In some embodiments, the hinge comprises amino acids Ala-Ser. In some other embodiments, the hinge comprises amino acids Ala-Ser and Thr-Lys-Gly. The hinge sequence can provide flexibility of binding to the target antigen, and balance between flexibility and optimal geometry.

In some embodiments, the single-chain variable fragment (scFv) described above includes a heavy chain variable domain and a light chain variable domain. In some embodiments, the heavy chain variable domain forms a disulfide bridge with the light chain variable domain to enhance stability of the scFv. For example, a disulfide bridge can be formed between the C44 residue of the heavy chain variable domain and the C100 residue of the light chain variable domain. In some embodiments, the heavy chain variable domain is linked to the light chain variable domain via a flexible linker. Any suitable linker can be used, for example, the (G4S)₄ linker. In some embodiments of the scFv, the heavy chain variable domain is positioned at the N-terminus of the light chain variable domain. In some embodiments of the scFv, the heavy chain variable domain is positioned at the C terminus of the light chain variable domain.

The multi-specific binding proteins can provide bivalent or monovalent engagement of BCMA. Bivalent engagement of BCMA by the multi-specific proteins can stabilize the BCMA on cell surface, and enhance cytotoxicity of NK cells towards the BCMA-expressing cells. Bivalent engagement of BCMA by the multi-specific proteins can confer stronger binding of the multi-specific proteins to the BCMA-expressing cells, thereby facilitating stronger cytotoxic response of NK cells towards the BCMA-expressing cells, especially towards BCMA-expressing cells expressing a low level of BCMA. Bivalent engagement of BCMA by the multi-specific proteins provided herein can also enhance cytotoxicity of cytotoxic T cells towards the BCMA-expressing cells. Bivalent engagement of BCMA by the multi-specific proteins can confer stronger binding of the multi-specific proteins to the BCMA-expressing cells, thereby facilitating stronger cytotoxic response of cytotoxic T cells towards the BCMA-expressing cells.

Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al, Nature, 406 (6793):267-273 (2000)). Based on the known domains, mutations can be selected to enhance or reduce the binding affinity to CD16, such as by using phage-displayed libraries or yeast surface-displayed cDNA libraries, or can be designed based on the known three-dimensional structure of the interaction.

In some embodiments, the antibody constant domain comprises a CH2 domain and a CH3 domain of an IgG antibody, for example, a human IgG1 antibody. In some embodiments, mutations are introduced in the antibody constant domain to enable heterodimerization with another antibody constant domain. For example, if the antibody constant domain is derived from the constant domain of a human IgG1, the antibody constant domain can comprise an amino acid sequence at least 90% identical to amino acids 234-332 of a human IgG1 antibody, and differs at one or more positions of Q347, Y349, L351, S354, E356, E357, K360, Q362, S364, T366, L368, K370, N390, K392, T394, D399, S400, D401, F405, Y407, K409, T411, and K439. All the amino acid positions in an Fc domain or hinge region disclosed herein are numbered according to EU numbering.

In some embodiments, the antibody constant domain can comprise an amino acid sequence at least 90% identical to amino acids 234-332 of a human IgG1 antibody, and differs by one or more substitutions of Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, Y407A, Y407I, Y407V, K409F, K409W, K409D, T411D, T411E, K439D, and K439E.

In a specific embodiment, the protein provide herein comprises a first amino acid chain comprising an amino acid sequence at least 90% identical to amino acids 234-332 of a human IgG1 antibody, comprising Y349C, K360E, K409W mutations, and a second amino acid chain comprising an amino acid sequence at least 90% identical to amino acids 234-332 of a human IgG1 antibody, comprising S354C, Q347R, D399V, and F405T mutations. In another embodiment, the protein provide herein comprises a first amino acid chain comprising an amino acid sequence at least 90% identical to amino acids 234-332 of a human IgG1 antibody, comprising S354C, K360E, K409W mutations, and a second amino acid chain comprising an amino acid sequence at least 90% identical to amino acids 234-332 of a human IgG1 antibody, comprising Y349C, Q347R, D399V, and F405T mutations.

Listed below are examples of the scFv linked to an antibody constant region that also includes mutations that enable heterodimerization of two polypeptide chains. The scFv containing a heavy chain variable domain (VH) and a light chain variable domain (VL) from NKG2D is used in preparing a multispecific protein of the present disclosure. Each sequence represents VL-(G4S)₄-VH-hinge (AS)-Fc containing heterodimerization mutations (underlined). VL and VH contain 100VL-44VH S-S bridge (underlined), and can be from any tumor targeting or NKG2D binding antibody. The Ala-Ser (AS, bolded & underlined) is included at the elbow hinge region sequence to balance between flexibility and optimal geometry. In certain embodiments, an additional sequence Thr-Lys-Gly can be added to the AS sequence at the hinge. (G4S)₄ linker is underlined in the sequences listed in the paragraph below.

A TriNKET of the present disclosure is an NKG2D-binding-F4-TriNKET-BCMA, A49-F4-TriNKET-BCMA, comprising a first polypeptide comprising the sequence of SEQ ID NO:162 (F4-BCMAFc-AJchainB-NKG2D-binding scFv), a second polypeptide comprising the sequence of SEQ ID NO:163 (Anti-BCMA HC-hinge-Fc), and a third and a fourth polypeptides each comprising the sequence of SEQ ID NO:165 (Anti-BCMA-Whole LC).

The first polypeptide, i.e., F4-BCMAFc-AJchainB-NKG2D-binding scFv (SEQ ID NO:162) and the third polypeptide, i.e. Anti-BCMA-Whole LC, forms a first BCMA-targeting Fab fragment (including a heavy chain portion comprising a heavy chain variable domain (VH) (SEQ ID NO:148) and a CH1 domain, and a light chain portion comprising a light chain variable domain (SEQ ID NO:152) and a light chain constant domain). F4-BCMAFc-AJchainB-NKG2D-binding scFv comprises the heavy chain portion (VH-CH1) connected to an Fc domain (hinge-CH2-CH3), which at the C-terminus of the Fc is linked to a single-chain variable fragment (scFv) that binds NKG2D. The scFv that binds NKG2D is represented by the amino acid sequence of SEQ ID NO:161, and includes a light chain variable domain (VL) (SEQ ID NO:98) linked to a heavy chain variable domain (VH) (SEQ ID NO:94) via a (G4S)₄ linker. As represented in SEQ ID NO:162, the C-terminus of the Fc domain is linked to the N-terminus of the VL (SEQ ID NO:98) domain using a short SGSGGGGS linker (SEQ ID NO:168).

NKG2D-binding scFv
(SEQ ID NO: 161)
DIQMTQSPSSVSASVGDRVTITCRASQGISSWLAWYQQKPGKAPKLLIYA
ASSLQSGVPSRFSGSGSGTDFTLTISSLQPEDFATYYCQQGVSFPRTFG [symbol]
GTKVEIKGGGGSGGGGSGGGGSGGGGSEVQLVESGGGLVKPGGSLRLSCA
ASGFTFSSYSMNWVRQAPGK [symbol] LEWVSSISSSSSYIYYADSVKGRFTISRD
NAKNSLYLQMNSLRAEDTAVYYCARGAPMGAAAGWFDPWGQGTLVTVSS
F4-BCMAFc-AJchainB-NKG2D-binding scFv
(SEQ ID NO: 162)
EVQLLESGGGLVQPGGSLRLSCAASGFTFSDNAMGWVRQAPGKGLEWVSA
ISGPGSSTYYADSVKGRFTISRDNSKNTLYLQMNSLRAEDTAVYYCAKVL
GWFDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPRVYTL
PP [symbol] RDELTKNQVSLTCLVKGFYPSDIAVEWESNGQPENNYKTTPPVLVSD
GSFTLYSKLTVDKSRWQQGNVFSCSVMHEALHNHYTQKSLSLSPGSGSGG
GGSDIQMTQSPSSVSASVGDRVTITCRASQGISSWLAWYQQKPGKAPKLL
IYAASSLQSGVPSRFSGSGSGTDFTLTISSLQPEDFATYYCQQGVSFPRT
FG [symbol] GTKVEIKGGGGSGGGGSGGGGSGGGGSEVQLVESGGGLVKPGGSLRL
SCAASGFTFSSYSMNWVRQAPGK [symbol] LEWVSSISSSSSYIYYADSVKGRFTI
SRDNAKNSLYLQMNSLRAEDTAVYYCARGAPMGAAAGWFDPWGQGTLVTV
SS

The scFv in the NKG2D-binding-F4-TriNKET-BCMA includes a light chain variable domain of an NKG2D-binding site connected to a heavy chain variable domain with a (G4S)₄ linker (represented as (VL(G4S)₄VH)). The light and the heavy variable domains of the scFv (SEQ ID NO:162) are connected as VL-(G4S)₄-VH; VL and VH contain 100VL-44VH S-S bridge (resulting from G100C and G44C substitutions, respectively) (cysteine residues are bold-italics-underlined). (G4S)₄ is the sequence in italics GGGGSGGGGSGGGGSGGGGS (SEQ ID NO:164) in SEQ ID NO:161 and SEQ ID NO:162. The Fc domain in SEQ ID NO:162 comprises an S354C substitution, which forms a disulfide bond with a Y349C substitution in another Fc domain (SEQ ID NO: 163, described below). The Fc domain in SEQ ID NO:162 includes Q347R, D399V, and F405T substitutions.

The second polypeptide, i.e. Anti-BCMA VH-CH1-Fc, and the fourth polypeptide, i.e. Anti-BCMA-Whole LC, forms a second BCMA-binding Fab fragment. Anti-BCMA VH-CH1-Fc includes a heavy chain portion comprising a heavy chain variable domain (SEQ ID NO:148) and a CH1 domain., wherein the heavy chain variable domain is connected to the CH1 domain, and the CH1 domain is connected to the Fc domain. Anti-BCMA-Whole LC includes a light chain portion comprising a light chain variable domain (SEQ ID NO:152) and a light chain constant domain.

Anti-BCMA VH-CH1-Fc
(SEQ ID NO: 163)
EVQLLESGGGLVQPGGSLRLSCAASGFTFSDNAMGWVRQAPGKGLEWVSA
ISGPGSSTYYADSVKGRFTISRDNSKNTLYLQMNSLRAEDTAVYYCAKVL
GWFDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQV [symbol] TL
PPSRDELTENQVSLTCLVKGFYPSDIAVEWESNGQPENNYKTTPPVLDSD
GSFFLYSWLTVDKSRWQQGNVFSCSVMHEALHNHYTQKSLSLSPG

SEQ ID NO:163 represents the heavy chain portion of the second anti-BCMA Fab fragment, which comprises a heavy chain variable domain (SEQ ID NO:148) of a BCMA-binding site and a CH1 domain, connected to an Fc domain (hinge-CH2-CH3). The Fc domain in SEQ ID NO:163 includes a Y349C substitution, which forms a disulfide bond with an S354C substitution in the CH3 domain of the Fc linked to the NKG2D-binding scFv (SEQ ID NO:162). In SEQ ID NO:163, the Fc domain also includes K360E and K409W substitutions.

SEQ ID NO:165 represents the light chain portion of a Fab fragments comprising a light chain variable domain (SEQ ID NO:152) of a BCMA-binding site and a light chain constant domain.

Anti-BCMA-Whole LC
(SEQ ID NO: 165)
EIVLTQSPGTLSLSPGERATLSCRASQSVSDEYLSWYQQKPGQAPRLLIH
SASTRATGIPDRFSGSGSGTDFTLAISRLEPEDFAVYYCQQYGYPPDFTF
GQGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVTH
QGLSSPVTKSFNRGEC

In an exemplary embodiment, the Fc domain linked to the NKG2D-binding scFv fragment comprises the mutations of K360E and K409W, and the Fc domain linked to the BCMA Fab fragment comprises matching mutations Q347R, D399V, and F405T for forming a heterodimer.

In an exemplary embodiment, the Fc domain linked to the NKG2D-binding scFv includes a Y349C substitution in the CH3 domain, which forms a disulfide bond with an S354C substitution on the Fc linked to the BCMA-binding Fab fragment.

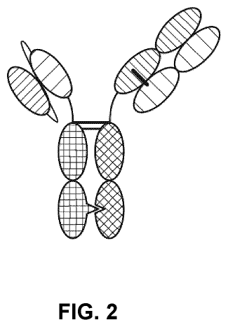

The F3 TriNKET is a heterodimeric, multi-specific antibody that includes three peptides: a first immunoglobulin heavy chain, a second immunoglobulin heavy chain and a immunoglobulin light chain (FIG. 2). The first immunoglobulin heavy chain includes, from N-terminus to C-terminus, a scFv, and a first Fc (CH2-CH3) domain, wherein the scFv is linked to the first Fc via either a linker or a hinge (FIG. 2). The second immunoglobulin heavy chain includes, from N-terminus to C-terminus, a (VH-CH1) domain, and a second Fc (CH2-CH3) domain, wherein the second Fc domain is linked via either a linker or a hinge at its N-terminus to the (VH-CH1) domain, and wherein the (VH-CH1) domain pairs with the light chain to form a Fab (FIG. 2). In certain embodiments, the scFv binds NKG2D, whereas the Fab binds BCMA. TriNKETs termed “NKG2D-binding-F3-TriNKET-BCMA” can refer to the TriNKETs depicted in FIG. 2. Another exemplary TriNKET of the present disclosure is NKG2D-binding-F3-TriNKET-BCMA, sequences of which are described below (CDRs (Kabat numbering) are underlined). In other embodiments, the scFv binds BCMA, whereas the Fab binds NKG2D.

An exemplary NKG2D-binding-F3-TriNKET-BCMA includes a BCMA-binding Fab fragment that includes a heavy chain portion comprising a heavy chain variable domain (SEQ ID NO:148) and a CH1 domain, and a light chain portion comprising a light chain variable domain (SEQ ID NO:152) and a light chain constant domain, wherein the heavy chain variable domain is connected to the CH1 domain, and the CH1 domain is connected to the Fc domain. NKG2D-binding-F3-TriNKET-BCMA also comprises an NKG2D-binding scFv linked to an Fc domain (SEQ ID NO: 166).

SEQ ID NO:163 represents an exemplary second immunoglobulin heavy chain of NKG2D-binding-F3-TriNKET-BCMA as depicted in FIG. 2, including the heavy chain portion of an anti-BCMA Fab fragment, which comprises a heavy chain variable domain (SEQ ID NO:148) of a BCMA-binding site and a CH1 domain, connected to an Fc domain. The Fc domain in SEQ ID NO:163 includes a Y349C substitution, which forms a disulfide bond with an S354C substitution in the CH3 domain of the Fc linked to the NKG2D-binding scFv (SEQ ID NO:166) for forming the NKG2D-binding-F3-TriNKET-BCMA. In SEQ ID NO:163, the Fc domain also includes K360E and K409W substitutions.

In an exemplary first immunoglobulin heavy chain of NKG2D-binding-F3-TriNKET-BCMA, the scFv in the NKG2D-binding-F3-TriNKET-BCMA includes a light chain variable domain of an NKG2D-binding site connected to a heavy chain variable domain with a (G4S)₄ linker (SEQ ID NO:164) (represented as (VL(G4S)₄VH)), which is linked to an Fc domain. In NKG2D-binding-F3-TriNKET-BCMA, the light and the heavy variable domains of the scFv (SEQ ID NO:161) are connected as VL-(G4S)₄-VH; VL and VH contain 100VL-44VH S-S bridge (resulting from G100C and G44C substitutions, respectively) (cysteine residues are bold-italics-underlined); and VH is connected to the Fc domain via an Ala-Ser.

SEQ ID NO:166 represents the full sequence of an NKG2D-binding scFv linked to an Fc domain via a hinge comprising Ala-Ser (scFv-Fc). The Fc domain linked to the scFv includes Q347R, D399V, and F405T substitutions.

F3-NKG2D-binding scFv-Fc-AJchainB
[V_{L}(G4S)₄V_{H})]
(SEQ ID NO: 166)
DIQMTQSPSSVSASVGDRVTITCRASQGISSWLAWYQQKPGKAPKLLIYA
ASSLQSGVPSRFSGSGSGTDFTLTISSLQPEDFATYYCQQGVSFPRTFG [symbol]
GTKVEIKGGGGSGGGGSGGGGSGGGGSEVQLVESGGGLVKPGGSLRLSCA
ASGFTFSSYSMNWVRQAPGK [symbol] LEWVSSISSSSSYIYYADSVKGRFTISRD
NAKNSLYLQMNSLRAEDTAVYYCARGAPMGAAAGWFDPWGQGTLVTVSSA
SDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPRVYTLPP [symbol] RDELTKNQVSLTCLV
KGFYPSDIAVEWESNGQPENNYKTTPPVLVSDGSFTLYSKLTVDKSRWQQ
GNVFSCSVMHEALHNHYTQKSLSLSPG

In an exemplary embodiment of an NKG2D-binding-F3-TriNKET-BCMA, the Fc domain of the first immunoglobulin heavy chain, which is linked to the NKG2D-binding scFv fragment comprises the mutations of K360E and K409W, and the Fc domain of the second immunoglobulin heavy chain, which is linked to the BCMA Fab fragment comprises matching mutations Q347R, D399V, and F405T for forming a heterodimer.

In an exemplary embodiment of an NKG2D-binding-F3-TriNKET-BCMA, the Fc domain of the first immunoglobulin heavy chain, which is linked to the NKG2D-binding scFv includes a Y349C substitution in the CH3 domain, which forms a disulfide bond with an S354C substitution on the Fc domain of the second immunoglobulin heavy chain, which is linked to the BCMA-binding Fab fragment.

The multi-specific binding proteins can bind to the NKG2D receptor-expressing cells, which can include but are not limited to NK cells, γδ T cells and CD8+αβ T cells. Upon NKG2D binding, the multi-specific binding proteins may block natural ligands, such as ULBP6 and MICA, from binding to NKG2D and activating NKG2D receptors.

The multi-specific binding proteins binds to cells expressing CD16, an Fc receptor on the surface of leukocytes including natural killer cells, macrophages, neutrophils, eosinophils, mast cells, and follicular dendritic cells.

A multi-specific binding protein described herein binds to NKG2D with an affinity of KD of 10 nM or lower, e.g., about 10 nM, about 9 nM, about 8 nM, about 7 nM, about 6 nM, about 5 nM, about 4.5 nM, about 4 nM, about 3.5 nM, about 3 nM, about 2.5 nM, about 2 nM, about 1.5 nM, about 1 nM, between about 0.5 nM-about 1 nM, about 1 nM-about 2 nM, about 2 nM-3 nM, about 3 nM-4 nM, about 4 nM-about 5 nM, about 5 nM-about 6 nM, about 6 nM-about 7 nM, about 7 nM-about 8 nM, about 8 nM-about 9 nM, about 9 nM-about 10 nM, about 1 nM-about 10 nM, about 2 nM-about 10 nM, about 3 nM-about 10 nM, about 4 nM-about 10 nM, about 5 nM-about 10 nM, about 6 nM-about 10 nM, about 7 nM-about 10 nM, or about 8 nM-about 10 nM.

Upon binding to the NKG2D receptor and CD16 receptor on natural killer cells, and a tumor-associated antigen on BCMA-expressing cells, the multi-specific binding proteins can engage more than one kind of NK-activating receptor, and may block the binding of natural ligands to NKG2D. In certain embodiments, the proteins can agonize NK cells in humans. In some embodiments, the proteins can agonize NK cells in humans and in other species such as rodents and cynomolgus monkeys.

Upon binding to the NKG2D receptor and CD16 receptor on cytotoxic T cells, and a tumor-associated antigen on BCMA-expressing cells, the multi-specific binding proteins can engage more than one kind of activating receptor, and may block the binding of natural ligands to NKG2D. In certain embodiments, the proteins can agonize cytotoxic T cells in humans. In some embodiments, the proteins can agonize cytotoxic T cells in humans and in other species such as rodents and cynomolgus monkeys.

(A) NKG2D-Binding Site

Table 1 lists peptide sequences of heavy chain variable domains and light chain variable domains that, in combination, can bind to NKG2D. In some embodiments, the heavy chain variable domain and the light chain variable domain are arranged in Fab format. In some embodiments, the heavy chain variable domain and the light chain variable domain are fused together to from an scFv.

The NKG2D binding domains can vary in their binding affinity to NKG2D, nevertheless, they can activate NKG2D expressing cells, such as NK cells and cytotoxic T cells.

Unless indicated otherwise, the CDR sequences provided in Table 1 are determined under Kabat.

TABLE 1
Heavy chain variable domains and light chain
variable domains of the NKG2D-binding site

| | Heavy chain | Light chain |
|---|---|---|
| | variable region amino | variable region |
| Clones | acid sequence | amino acid sequence |
| ADI- | QVQLQQWGAGLLKPSETLSLTCAVYG | DIQMTQSPSTLSASVGDRVTITC |
| 27705 | GSFSGYYWSWIRQPPGKGLEWIGEID | RASQSISSWLAWYQQKPGKAPKL |
| | HSGSTNYNPSLKSRVTISVDTSKNQF | LIYKASSLESGVPSRFSGSGSGT |
| | SLKLSSVTAADTAVYYCARARGPWSF | EFTLTISSLQPDDFATYYCQQYN |
| | DPWGQGTLVTVSS | SYPITFGGGTKVEIK |
| | (SEQ ID NO: 1) | (SEQ ID NO: 2) |
| | CDR1 (SEQ ID NO: 3) - | |
| | GSFSGYYWS | |
| | CDR2 (SEQ ID NO: 4) - | |
| | EIDHSGSTNYNPSLKS | |
| | CDR3 (SEQ ID NO: 5) - | |
| | ARARGPWSFDP | |
| ADI- | QVQLQQWGAGLLKPSETLSLTCAVYG | EIVLTQSPGTLSLSPGERATLSC |
| 27724 | GSFSGYYWSWIRQPPGKGLEWIGEID | RASQSVSSSYLAWYQQKPGQAPR |
| | HSGSTNYNPSLKSRVTISVDTSKNQF | LLIYGASSRATGIPDRFSGSGSG |
| | SLKLSSVTAADTAVYYCARARGPWSF | TDFTLTISRLEPEDFAVYYCQQY |
| | DPWGQGTLVTVSS | GSSPITFGGGTKVEIK |
| | (SEQ ID NO: 6) | (SEQ ID NO: 7) |
| ADI- | QVQLQQWGAGLLKPSETLSLTCAVYG | DIQMTQSPSTLSASVGDRVTITC |
| 27740 | GSFSGYYWSWIRQPPGKGLEWIGEID | RASQSIGSWLAWYQQKPGKAPKL |
| (A40) | HSGSTNYNPSLKSRVTISVDTSKNQF | LIYKASSLESGVPSRFSGSGSGT |
| | SLKLSSVTAADTAVYYCARARGPWSF | EFTLTISSLQPDDFATYYCQQYH |
| | DPWGQGTLVTVSS | SFYTFGGGTKVEIK |
| | (SEQ ID NO: 8) | (SEQ ID NO: 9) |
| ADI- | QVQLQQWGAGLLKPSETLSLTCAVYG | DIQMTQSPSTLSASVGDRVTITC |
| 27741 | GSFSGYYWSWIRQPPGKGLEWIGEID | RASQSIGSWLAWYQQKPGKAPKL |
| | HSGSTNYNPSLKSRVTISVDTSKNQF | LIYKASSLESGVPSRFSGSGSGT |
| | SLKLSSVTAADTAVYYCARARGPWSF | EFTLTISSLQPDDFATYYCQQSN |
| | DPWGQGTLVTVSS | SYYTFGGGTKVEIK |
| | (SEQ ID NO: 10) | (SEQ ID NO: 11) |
| ADI- | QVQLQQWGAGLLKPSETLSLTCAVYG | DIQMTQSPSTLSASVGDRVTITC |
| 27743 | GSFSGYYWSWIRQPPGKGLEWIGEID | RASQSISSWLAWYQQKPGKAPKL |
| | HSGSTNYNPSLKSRVTISVDTSKNQF | LIYKASSLESGVPSRFSGSGSGT |
| | SLKLSSVTAADTAVYYCARARGPWSF | EFTLTISSLQPDDFATYYCQQYN |
| | DPWGQGTLVTVSS | SYPTFGGGTKVEIK |
| | (SEQ ID NO: 12) | (SEQ ID NO: 13) |
| ADI- | QVQLQQWGAGLLKPSETLSLTCAVYG | ELQMTQSPSSLSASVGDRVTITC |
| 28153 | GSFSGYYWSWIRQPPGKGLEWIGEID | RTSQSISSYLNWYQQKPGQPPKL |
| | HSGSTNYNPSLKSRVTISVDTSKNQF | LIYWASTRESGVPDRFSGSGSGT |
| | SLKLSSVTAADTAVYYCARARGPWGF | DFTLTISSLOPEDSATYYCQQSY |
| | DPWGQGTLVTVSS | DIPYTFGQGTKLEIK |
| | (SEQ ID NO: 14) | (SEQ ID NO: 15) |
| ADI- | QVOLQQWGAGLLKPSETLSLTCAVYG | DIQMTQSPSTLSASVGDRVTITC |
| 28226 | GSFSGYYWSWIRQPPGKGLEWIGEID | RASQSISSWLAWYQQKPGKAPKL |
| (C26) | HSGSTNYNPSLKSRVTISVDTSKNQF | LIYKASSLESGVPSRFSGSGSGT |
| | SLKLSSVTAADTAVYYCARARGPWSF | EFTLTISSLQPDDFATYYCQQYG |
| | DPWGQGTLVTVSS | SFPITFGGGTKVEIK |
| | (SEQ ID NO: 16) | (SEQ ID NO: 17) |
| ADI- | QVOLQQWGAGLLKPSETLSLTCAVYG | DIQMTQSPSTLSASVGDRVTITC |
| 28154 | GSFSGYYWSWIRQPPGKGLEWIGEID | RASQSISSWLAWYQQKPGKAPKL |
| | HSGSTNYNPSLKSRVTISVDTSKNQF | LIYKASSLESGVPSRFSGSGSGT |
| | SLKLSSVTAADTAVYYCARARGPWSF | DFTLTISSLQPDDFATYYCQQSK |
| | DPWGQGTLVTVSS | EVPWTFGQGTKVEIK |
| | (SEQ ID NO: 18) | (SEQ ID NO: 19) |
| ADI- | QVQLQQWGAGLLKPSETLSLTCAVYG | DIQMTQSPSTLSASVGDRVTITC |
| 29399 | GSFSGYYWSWIRQPPGKGLEWIGEID | RASQSISSWLAWYQQKPGKAPKL |
| | HSGSTNYNPSLKSRVTISVDTSKNQF | LIYKASSLESGVPSRFSGSGSGT |
| | SLKLSSVTAADTAVYYCARARGPWSF | EFTLTISSLQPDDFATYYCQQYN |
| | DPWGQGTLVTVSS | SFPTFGGGTKVEIK |
| | (SEQ ID NO: 20) | (SEQ ID NO: 21) |
| ADI- | QVQLQQWGAGLLKPSETLSLTCAVYG | DIQMTQSPSTLSASVGDRVTITC |
| 29401 | GSFSGYYWSWIRQPPGKGLEWIGEID | RASQSIGSWLAWYQQKPGKAPKL |
| | HSGSTNYNPSLKSRVTISVDTSKNQF | LIYKASSLESGVPSRFSGSGSGT |
| | SLKLSSVTAADTAVYYCARARGPWSF | EFTLTISSLQPDDFATYYCQQYD |
| | DPWGQGTLVTVSS | IYPTFGGGTKVEIK |
| | (SEQ ID NO: 22) | (SEQ ID NO: 23) |
| ADI- | QVQLQQWGAGLLKPSETLSLTCAVYG | DIQMTQSPSTLSASVGDRVTITC |
| 29403 | GSFSGYYWSWIRQPPGKGLEWIGEID | RASQSISSWLAWYQQKPGKAPKL |
| | HSGSTNYNPSLKSRVTISVDTSKNQF | LIYKASSLESGVPSRFSGSGSGT |
| | SLKLSSVTAADTAVYYCARARGPWSF | EFTLTISSLQPDDFATYYCQQYD |
| | DPWGQGTLVTVSS | SYPTFGGGTKVEIK |
| | (SEQ ID NO: 24) | (SEQ ID NO: 25) |
| ADI- | QVQLQQWGAGLLKPSETLSLTCAVYG | DIQMTQSPSTLSASVGDRVTITC |
| 29405 | GSFSGYYWSWIRQPPGKGLEWIGEID | RASQSISSWLAWYQQKPGKAPKL |
| | HSGSTNYNPSLKSRVTISVDTSKNQF | LIYKASSLESGVPSRFSGSGSGT |
| | SLKLSSVTAADTAVYYCARARGPWSF | EFTLTISSLQPDDFATYYCQQYG |
| | DPWGQGTLVTVSS | SFPTFGGGTKVEIK |
| | (SEQ ID NO: 26) | (SEQ ID NO: 27) |
| ADI- | QVQLQQWGAGLLKPSETLSLTCAVYG | DIQMTQSPSTLSASVGDRVTITC |
| 29407 | GSFSGYYWSWIRQPPGKGLEWIGEID | RASQSISSWLAWYQQKPGKAPKL |
| | HSGSTNYNPSLKSRVTISVDTSKNQF | LIYKASSLESGVPSRFSGSGSGT |
| | SLKLSSVTAADTAVYYCARARGPWSF | EFTLTISSLQPDDFATYYCQQYQ |
| | DPWGQGTLVTVSS | SFPTFGGGTKVEIK |
| | (SEQ ID NO: 28) | (SEQ ID NO: 29) |
| ADI- | QVOLQQWGAGLLKPSETLSLTCAVYG | DIQMTQSPSTLSASVGDRVTITC |
| 29419 | GSFSGYYWSWIRQPPGKGLEWIGEID | RASQSISSWLAWYQQKPGKAPKL |
| | HSGSTNYNPSLKSRVTISVDTSKNQF | LIYKASSLESGVPSRFSGSGSGT |
| | SLKLSSVTAADTAVYYCARARGPWSF | EFTLTISSLQPDDFATYYCQQYS |
| | DPWGQGTLVTVSS | SFSTFGGGTKVEIK |
| | (SEQ ID NO: 30) | (SEQ ID NO: 31) |
| ADI- | QVOLQQWGAGLLKPSETLSLTCAVYG | DIQMTQSPSTLSASVGDRVTITC |
| 29421 | GSFSGYYWSWIRQPPGKGLEWIGEID | RASQSISSWLAWYQQKPGKAPKL |
| | HSGSTNYNPSLKSRVTISVDTSKNQF | LIYKASSLESGVPSRFSGSGSGT |
| | SLKLSSVTAADTAVYYCARARGPWSF | EFTLTISSLQPDDFATYYCQQYE |
| | DPWGQGTLVTVSS | SYSTFGGGTKVEIK |
| | (SEQ ID NO: 32) | (SEQ ID NO: 33) |
| ADI- | QVQLQQWGAGLLKPSETLSLTCAVYG | DIQMTQSPSTLSASVGDRVTITC |
| 29424 | GSFSGYYWSWIRQPPGKGLEWIGEID | RASQSISSWLAWYQQKPGKAPKL |
| | HSGSTNYNPSLKSRVTISVDTSKNQF | LIYKASSLESGVPSRFSGSGSGT |
| | SLKLSSVTAADTAVYYCARARGPWSF | EFTLTISSLQPDDFATYYCQQYD |
| | DPWGQGTLVTVSS | SFITFGGGTKVEIK |
| | (SEQ ID NO: 34) | (SEQ ID NO: 35) |
| ADI- | QVQLQQWGAGLLKPSETLSLTCAVYG | DIQMTQSPSTLSASVGDRVTITC |
| 29425 | GSFSGYYWSWIRQPPGKGLEWIGEID | RASQSISSWLAWYQQKPGKAPKL |
| | HSGSTNYNPSLKSRVTISVDTSKNQF | LIYKASSLESGVPSRFSGSGSGT |
| | SLKLSSVTAADTAVYYCARARGPWSF | EFTLTISSLQPDDFATYYCQQYQ |
| | DPWGQGTLVTVSS | SYPTFGGGTKVEIK |
| | (SEQ ID NO: 36) | (SEQ ID NO: 37) |
| ADI- | QVOLQQWGAGLLKPSETLSLTCAVYG | DIQMTQSPSTLSASVGDRVTITC |
| 29426 | GSFSGYYWSWIRQPPGKGLEWIGEID | RASQSIGSWLAWYQQKPGKAPKL |
| | HSGSTNYNPSLKSRVTISVDTSKNQF | LIYKASSLESGVPSRFSGSGSGT |
| | SLKLSSVTAADTAVYYCARARGPWSF | EFTLTISSLQPDDFATYYCQQYH |
| | DPWGQGTLVTVSS | SFPTFGGGTKVEIK |
| | (SEQ ID NO: 38) | (SEQ ID NO: 39) |
| ADI- | QVOLQQWGAGLLKPSETLSLTCAVYG | DIQMTQSPSTLSASVGDRVTITC |
| 29429 | GSFSGYYWSWIRQPPGKGLEWIGEID | RASQSIGSWLAWYQQKPGKAPKL |
| | HSGSTNYNPSLKSRVTISVDTSKNQF | LIYKASSLESGVPSRFSGSGSGT |
| | SLKLSSVTAADTAVYYCARARGPWSF | EFTLTISSLQPDDFATYYCQQYE |
| | DPWGQGTLVTVSS | LYSYTFGGGTKVEIK |
| | (SEQ ID NO: 40) | (SEQ ID NO: 41) |
| ADI- | QVQLQQWGAGLLKPSETLSLTCAVYG | DIQMTQSPSTLSASVGDRVTITC |
| 29447 | GSFSGYYWSWIRQPPGKGLEWIGEID | RASQSISSWLAWYQQKPGKAPKL |
| (F47) | HSGSTNYNPSLKSRVTISVDTSKNQF | LIYKASSLESGVPSRFSGSGSGT |
| | SLKLSSVTAADTAVYYCARARGPWSF | EFTLTISSLQPDDFATYYCQQYD |
| | DPWGQGTLVTVSS | TFITFGGGTKVEIK |
| | (SEQ ID NO: 42) | (SEQ ID NO: 43) |
| ADI- | QVQLVQSGAEVKKPGSSVKVSCKASG | DIVMTQSPDSLAVSLGERATINC |
| 27727 | GTFSSYAISWVRQAPGQGLEWMGGII | KSSQSVLYSSNNKNYLAWYQQKP |
| | PIFGTANYAQKFQGRVTITADESTST | GQPPKLLIYWASTRESGVPDRFS |
| | AYMELSSLRSEDTAVYYCARGDSSIR | GSGSGTDFTLTISSLQAEDVAVY |
| | HAYYYYGMDVWGQGTTVTVSS | YCQQYYSTPITFGGGTKVEIK |
| | (SEQ ID NO: 44) | (SEQ ID NO: 48) |
| | CDR1 (SEQ ID NO: 45) - | CDR1 (SEQ ID NO: 49) - |
| | GTFSSYAIS (non-Kabat) or | KSSQSVLYSSNNKNYLA |
| | SYAIS (SED ID NO: 181) | CDR2 (SEQ ID NO: 50) - |
| | CDR2 (SEQ ID NO: 46) - | WASTRES |
| | GIIPIFGTANYAQKFQG | CDR3 (SEQ ID NO: 51) - |
| | CDR3 (SEQ ID NO: 47) - | QQYYSTPIT |
| | ARGDSSIRHAYYYYGMDV (non- | |
| | Kabat) or GDSSIRHAYYYYGMDV | |
| | (SEQ ID NO: 182) | |
| ADI- | QLQLQESGPGLVKPSETLSLTCTVSG | EIVLTQSPATLSLSPGERATLSC |
| 29443 | GSISSSSYYWGWIRQPPGKGLEWIGS | RASQSVSRYLAWYQQKPGQAPRL |
| (F43) | IYYSGSTYYNPSLKSRVTISVDTSKN | LIYDASNRATGIPARFSGSGSGT |
| | QFSLKLSSVTAADTAVYYCARGSDRF | DFTLTISSLEPEDFAVYYCQQFD |
| | HPYFDYWGQGTLVTVSS | TWPPTFGGGTKVEIK |
| | (SEQ ID NO: 52) | (SEQ ID NO: 56) |
| | CDR1 (SEQ ID NO: 53) - | CDR1 (SEQ ID NO: 57) - |
| | GSISSSSYYWG (non-Kabat) or | RASQSVSRYLA |
| | SSSYYWG (SEQ ID NO: 183) | CDR2 (SEQ ID NO: 58) - |
| | CDR2 (SEQ ID NO: 54) | DASNRAT |
| | SIYYSGSTYYNPSLKS | CDR3 (SEQ ID NO: 59) - |
| | CDR3 (SEQ ID NO: 55) | QQFDTWPPT |
| | ARGSDRFHPYFDY (non-Kabat) | |
| | or GSDRFHPYFDY (SEQ ID | |
| | NO: 184) | |
| ADI- | QVQLQQWGAGLLKPSETLSLTCAVYG | DIQMTQSPSTLSASVGDRVTITC |
| 29404 | GSFSGYYWSWIRQPPGKGLEWIGEID | RASQSISSWLAWYQQKPGKAPKL |
| (F04) | HSGSTNYNPSLKSRVTISVDTSKNQF | LIYKASSLESGVPSRFSGSGSGT |
| | SLKLSSVTAADTAVYYCARARGPWSF | EFTLTISSLQPDDFATYYCEQYD |
| | DPWGQGTLVTVSS | SYPTFGGGTKVEIK |
| | (SEQ ID NO: 60) | (SEQ ID NO: 61) |
| ADI- | QVQLVQSGAEVKKPGSSVKVSCKASG | DIVMTQSPDSLAVSLGERATINC |
| 28200 | GTFSSYAISWVRQAPGOGLEWMGGII | ESSQSLLNSGNQKNYLTWYQQKP |
| | PIFGTANYAQKFQGRVTITADESTST | GQPPKPLIYWASTRESGVPDRFS |
| | AYMELSSLRSEDTAVYYCARRGRKAS | GSGSGTDFTLTISSLQAEDVAVY |
| | GSFYYYYGMDVWGQGTTVTVSS | YCONDYSYPYTFGQGTKLEIK |
| | (SEQ ID NO: 62) | (SEQ ID NO: 66) |
| | CDR1 (SEQ ID NO: 63) - | CDR1 (SEQ ID NO: 67) - |
| | GTFSSYAIS (non-Kabat) or | ESSQSLLNSGNQKNYLT |
| | SYAIS (SEQ ID NO: 181) | CDR2 (SEQ ID NO: 68) - |
| | CDR2 (SEQ ID NO: 64) - | WASTRES |
| | GIIPIFGTANYAQKFQG | CDR3 (SEQ ID NO: 69) - |
| | CDR3 (SEQ ID NO: 65) - | QNDYSYPYT |
| | ARRGRKASGSFYYYYGMDV | |
| ADI- | QVOLVOSGAEVKKPGASVKVSCKASG | EIVMTQSPATLSVSPGERATLSC |
| 29379 | YTFTSYYMHWVRQAPGQGLEWMGIIN | RASQSVSSNLAWYQQKPGQAPRL |
| (E79) | PSGGSTSYAQKFQGRVTMTRDTSTST | LIYGASTRATGIPARFSGSGSGT |
| | VYMELSSLRSEDTAVYYCARGAPNYG | EFTLTISSLQSEDFAVYYCQQYD |
| | DTTHDYYYMDVWGKGTTVTVSS | DWPFTFGGGTKVEIK |
| | (SEQ ID NO: 70) | (SEQ ID NO: 74) |
| | CDR1 (SEQ ID NO: 71) - | CDR1 (SEQ ID NO: 75) - |
| | YTFTSYYMH (non-Kabat) or | RASQSVSSNLA |
| | SYYMH (SEQ ID NO: 185) | CDR2 (SEQ ID NO: 76) - |
| | CDR2 (SEQ ID NO: 72) - | GASTRAT |
| | IINPSGGSTSYAQKFQG | CDR3 (SEQ ID NO: 77) - |
| | CDR3 (SEQ ID NO: 73) - | QQYDDWPFT |
| | ARGAPNYGDTTHDYYYMDV (non- | |
| | Kabat) or | |
| | GAPNYGDTTHDYYYMDV (SEQ ID | |
| | NO: 159) | |
| ADI- | QVQLVQSGAEVKKPGASVKVSCKASG | EIVLTQSPGTLSLSPGERATLSC |
| 29463 | YTFTGYYMHWVRQAPGQGLEWMGWIN | RASQSVSSNLAWYQQKPGQAPRL |
| (F63) | PNSGGTNYAQKFQGRVTMTRDTSIST | LIYGASTRATGIPARFSGSGSGT |
| | AYMELSRLRSDDTAVYYCARDTGEYY | EFTLTISSLQSEDFAVYYCQQDD |
| | DTDDHGMDVWGQGTTVTVSS | YWPPTFGGGTKVEIK |
| | (SEQ ID NO: 78) | (SEQ ID NO: 82) |
| | CDR1 (SEQ ID NO: 79) - | CDR1 (SEQ ID NO: 83) - |
| | YTFTGYYMH (non-Kabat) or | RASQSVSSNLA |
| | GYYMH (SEQ ID NO: 186) | CDR2 (SEQ ID NO: 84) - |
| | CDR2 (SEQ ID NO: 80) - | GASTRAT |
| | WINPNSGGTNYAQKFQG | CDR3 (SEQ ID NO: 85) - |
| | CDR3 (SEQ ID NO: 81) - | QQDDYWPPT |
| | ARDTGEYYDTDDHGMDV (non- | |
| | Kabat) or DTGEYYDTDDHGMDV | |
| | (SEQ ID NO: 187) | |
| ADI- | EVQLLESGGGLVQPGGSLRLSCAASG | DIQMTQSPSSVSASVGDRVTITC |
| 27744 | FTFSSYAMSWVROAPGKGLEWVSAIS | RASQGIDSWLAWYQQKPGKAPKL |
| (A44) | GSGGSTYYADSVKGRFTISRDNSKNT | LIYAASSLOSGVPSRFSGSGSGT |
| | LYLQMNSLRAEDTAVYYCAKDGGYYD | DFTLTISSLQPEDFATYYCQQGV |
| | SGAGDYWGQGTLVTVSS | SYPRTFGGGTKVEIK |
| | (SEQ ID NO: 86) | (SEQ ID NO: 90) |
| | CDR1 (SEQ ID NO: 87) | CDR1 (SEQ ID NO: 91) - |
| | FTFSSYAMS (non-Kabat) or | RASQGIDSWLA |
| | SYAMS (SEQ ID NO: 188) | CDR2 (SEQ ID NO: 92) - |
| | CDR2 (SEQ ID NO: 88) - | AASSLOS |
| | AISGSGGSTYYADSVKG | CDR3 (SEQ ID NO: 93) - |
| | CDR3 (SEQ ID NO: 89) - | QQGVSYPRT |
| | AKDGGYYDSGAGDY (non-Kabat) | |
| | or DGGYYDSGAGDY (SEQ ID | |
| | NO: 189) | |
| ADI- | EVQLVESGGGLVKPGGSLRLSCAASG | DIQMTQSPSSVSASVGDRVTITC |
| 27749 | FTFSSYSMNWVRQAPGKGLEWVSSIS | RASQGISSWLAWYQQKPGKAPKL |
| (A49) | SSSSYIYYADSVKGRFTISRDNAKNS | LIYAASSLQSGVPSRFSGSGSGT |
| | LYLQMNSLRAEDTAVYYCARGAPMGA | DFTLTISSLOPEDFATYYCQQGV |
| | AAGWFDPWGQGTLVTVSS | SFPRTFGGGTKVEIK |
| | (SEQ ID NO: 94) | (SEQ ID NO: 98) |
| | CDR1 (SEQ ID NO: 95) - | CDR1 (SEQ ID NO: 99) - |
| | FTFSSYSMN (non-Kabat) or | RASQGISSWLA |
| | SYSMN (SEQ ID NO: 190) | CDR2 (SEQ ID NO: 100) - |
| | CDR2 (SEQ ID NO: 96) - | AASSLQS |
| | SISSSSSYIYYADSVKG | CDR3 (SEQ ID NO: 101) - |
| | CDR3 (SEQ ID NO: 97) - | QQGVSFPRT |
| | ARGAPMGAAAGWFDP (non- | |
| | Kabat) or GAPMGAAAGWFDP | |
| | (SEQ ID NO: 191) | |
| ADI- | QVQLVQSGAEVKKPGASVKVSCKASG | EIVLTQSPATLSLSPGERATLSC |
| 29378 | YTFTSYYMHWVRQAPGQGLEWMGIIN | RASQSVSSYLAWYQQKPGQAPRL |
| (E78) | PSGGSTSYAQKFQGRVTMTRDTSTST | LIYDASNRATGIPARFSGSGSGT |
| | VYMELSSLRSEDTAVYYCAREGAGFA | DFTLTISSLEPEDFAVYYCQQSD |
| | YGMDYYYMDVWGKGTTVTVSS | NWPFTFGGGTKVEIK |
| | (SEQ ID NO: 102) | (SEQ ID NO: 106) |
| | CDR1 (SEQ ID NO: 103) - | CDR1 (SEQ ID NO: 107) - |
| | YTFTSYYMH (non-Kabat) or | RASQSVSSYLA |
| | SYYMH (SEQ ID NO: 185) | CDR2 (SEQ ID NO: 108) - |
| | CDR2 (SEQ ID NO: 104) - | DASNRAT |
| | IINPSGGSTSYAQKFQG | CDR3 (SEQ ID NO: 109) - |
| | CDR3 (SEQ ID NO: 105) - | QQSDNWPFT |
| | AREGAGFAYGMDYYYMDV or | |
| | EGAGFAYGMDYYYMDV (SEQ ID | |
| | NO: 192) | |
| A49MI | EVQLVESGGGLVKPGGSLRLSCAASG | DIQMTQSPSSVSASVGDRVTITC |
| | FTFSSYSMNWVRQAPGKGLEWVSSIS | RASQGISSWLAWYQQKPGKAPKL |
| | SSSSYIYYADSVKGRFTISRDNAKNS | LIYAASSLQSGVPSRFSGSGSGT |
| | LYLQMNSLRAEDTAVYYCARGAPIGA | DFTLTISSLOPEDFATYYCQQGV |
| | AAGWFDPWGQGTLVTVSS (SEQ ID | SFPRTFGGGTKVEIK |
| | NO: 169) | (SEQ ID NO: 98) |
| | CDR1 (SEQ ID NO:95) - | CDR1 (SEQ ID NO: 99) - |
| | FTFSSYSMN (non-Kabat) or | RASQGISSWLA |
| | SYSMN (SEQ ID NO: 190) | CDR2 (SEQ ID NO: 100) - |
| | CDR2 (SEQ ID NO: 96) - | AASSLQS |
| | SISSSSSYIYYADSVKG | CDR3 (SEQ ID NO: 101) - |
| | CDR3: (SEQ ID NO: 170) - | QQGVSFPRT |
| | ARGAPIGAAAGWFDP (non- | |
| | Kabat) or GAPIGAAAGWFDP | |
| | (SEQ ID NO: 193) | |
| A49MQ | EVQLVESGGGLVKPGGSLRLSCAASG | DIQMTQSPSSVSASVGDRVTITC |
| | FTFSSYSMNWVRQAPGKGLEWVSSIS | RASQGISSWLAWYQQKPGKAPKL |
| | SSSSYIYYADSVKGRFTISRDNAKNS | LIYAASSLQSGVPSRFSGSGSGT |
| | LYLQMNSLRAEDTAVYYCARGAPQGA | DFTLTISSLQPEDFATYYCQQGV |
| | AAGWFDPWGQGTLVTVSS | SFPRTFGGGTKVEIK |
| | (SEQ ID NO: 171) | (SEQ ID NO: 98) |
| | CDR1 (SEQ ID NO: 95) - | CDR1 (SEQ ID NO: 99) - |
| | FTFSSYSMN (non-Kabat) or | RASQGISSWLA |
| | SYSMN (SEQ ID NO: 190) | CDR2 (SEQ ID NO: 100) - |
| | CDR2 (SEQ ID NO : 96) - | AASSLQS |
| | SISSSSSYIYYADSVKG | CDR3 (SEQ ID NO: 101) - |
| | CDR3 (SEQ ID NO: 172) - | QQGVSFPRT |
| | ARGAPQGAAAGWFDP (non- | |
| | Kabat) or GAPQGAAAGWFDP | |
| | (SEQ ID NO: 194) | |
| A49ML | EVQLVESGGGLVKPGGSLRLSCAASG | DIQMTQSPSSVSASVGDRVTITC |
| | FTFSSYSMNWVRQAPGKGLEWVSSIS | RASQGISSWLAWYQQKPGKAPKL |
| | SSSSYIYYADSVKGRFTISRDNAKNS | LIYAASSLQSGVPSRFSGSGSGT |
| | LYLQMNSLRAEDTAVYYCARGAPLGA | DFTLTISSLQPEDFATYYCQQGV |
| | AAGWFDPWGQGTLVTVSS | SFPRTFGGGTKVEIK |
| | (SEQ ID NO: 173) | (SEQ ID NO: 98) |
| | CDR1 (SEQ ID NO: 95) - | CDR1 (SEQ ID NO: 99) - |
| | FTFSSYSMN (non-Kabat) or | RASQGISSWLA |
| | SYSMN (SEQ ID NO: 190) | CDR2 (SEQ ID NO: 100) - |
| | CDR2 (SEQ ID NO: 96) - | AASSLOS |
| | SISSSSSYIYYADSVKG | 02 |
| | CDR3 (SEQ ID NO: 174) - | CDR3 (SEQ ID NO: 101) - |
| | ARGAPLGAAAGWFDP (non- | QQGVSFPRT |
| | Kabat) or GAPLGAAAGWFDP | |
| | (SEQ ID NO: 195) | |
| A49MF | EVQLVESGGGLVKPGGSLRLSCAASG | DIQMTQSPSSVSASVGDRVTITC |
| | FTFSSYSMNWVRQAPGKGLEWVSSIS | RASQGISSWLAWYQQKPGKAPKL |
| | SSSSYIYYADSVKGRFTISRDNAKNS | LIYAASSLQSGVPSRFSGSGSGT |
| | LYLQMNSLRAEDTAVYYCARGAPFGA | DFTLTISSLOPEDFATYYCQQGV |
| | AAGWFDPWGQGTLVTVSS | SFPRTFGGGTKVEIK |
| | (SEQ ID NO: 175) | (SEQ ID NO: 98) |
| | CDR1 (SEQ ID NO: 95) - | CDR1 (SEQ ID NO: 99) - |
| | FTESSYSMN (non-Kabat) or | RASQGISSWLA |
| | SYSMN (SEQ ID NO: 190) | CDR2 (SEQ ID NO: 100) - |
| | CDR2 (SEQ ID NO: 96) - | AASSLQS |
| | SISSSSSYIYYADSVKG | CDR3 (SEQ ID NO: 101) - |
| | CDR3 (SEQ ID NO:176) - | QQGVSFPRT |
| | ARGAPFGAAAGWFDP (non- | |
| | Kabat) or GAPFGAAAGWFDP | |
| | (SEQ ID NO: 196) | |
| A49MV | EVQLVESGGGLVKPGGSLRLSCAASG | DIQMTQSPSSVSASVGDRVTITC |
| | FTFSSYSMNWVRQAPGKGLEWVSSIS | RASQGISSWLAWYQQKPGKAPKL |
| | SSSSYIYYADSVKGRFTISRDNAKNS | LIYAASSLQSGVPSRFSGSGSGT |
| | LYLQMNSLRAEDTAVYYCARGAPVGA | DFTLTISSLQPEDFATYYCQQGV |
| | AAGWFDPWGQGTLVTVSS | SFPRTFGGGTKVEIK |
| | (SEQ ID NO: 177) | (SEQ ID NO: 98) |
| | CDR1 (SEQ ID NO: 95) | CDR1 (SEQ ID NO: 99) - |
| | FTFSSYSMN (non-Kabat) or | RASQGISSWLA |
| | SYSMN (SEQ ID NO: 190) | CDR2 (SEQ ID NO: 100) - |
| | CDR2 (SEQ ID NO: 96) - | AASSLQS |
| | SISSSSSYIYYADSVKG | CDR3 (SEQ ID NO: 101) |
| | CDR3 (SEQ ID NO: 178) - | QQGVSFPRT |
| | ARGAPVGAAAGWFDP (non- | |
| | Kabat) or GAPVGAAAGWFDP | |
| | (SEQ ID NO: 197) | |
| A49- | EVQLVESGGGLVKPGGSLRLSCAASG | DIQMTQSPSSVSASVGDRVTITC |
| consensus | FTFSSYSMNWVRQAPGKGLEWVSSIS | RASQGISSWLAWYQQKPGKAPKL |
| | SSSSYIYYADSVKGRFTISRDNAKNS | LIYAASSLQSGVPSRFSGSGSGT |
| | LYLQMNSLRAEDTAVYYCARGAPXGA | DFTLTISSLOPEDFATYYCQQGV |
| | AAGWFDPWGQGTLVTVSS, | SFPRTFGGGTKVEIK |
| | wherein X is M, L, I, V, | (SEQ ID NO: 98) |
| | Q, or F | CDR1 (SEQ ID NO: 99) - |
| | (SEQ ID NO: 179) | RASQGISSWLA |
| | CDR1 (SEQ ID NO: 95) | CDR2 (SEQ ID NO: 100) - |
| | FTFSSYSMN (non-Kabat) or | AASSLOS |
| | SYSMN (SEQ ID NO: 190) | CDR3 (SEQ ID NO: 101) - |
| | CDR2 (SEQ ID NO: 96) | QQGVSFPRT |
| | SISSSSSYIYYADSVKG | |
| | CDR3 (SEQ ID NO: 180) - | |
| | ARGAPXGAAAGWFDP, wherein X | |
| | is M, L, I, V, Q, or F | |
| | (non-Kabat) or | |
| | GAPXGAAAGWFDP, wherein X | |
| | is M, L, I, V, Q, or F | |
| | (SEQ ID NO: 160) | |

Alternatively, a heavy chain variable domain represented by SEQ ID NO:110 can be paired with a light chain variable domain represented by SEQ ID NO:111 to form an antigen-binding site that can bind to NKG2D, as illustrated in U.S. Pat. No. 9,273,136.

SEQ ID NO: 110
QVQLVESGGGLVKPGGSLRLSCAASGFTFSSYGMHWVRQAPGKGLEWVAF
IRYDGSNKYYADSVKGRFTISRDNSKNTLYLQMNSLRAEDTAVYYCAKDR
GLGDGTYFDYWGQGTTVTVSS
SEQ ID NO: 111
QSALTQPASVSGSPGQSITISCSGSSSNIGNNAVNWYQQLPGKAPKLLIY
YDDLLPSGVSDRFSGSKSGTSAFLAISGLQSEDEADYYCAAWDDSLNGPV
FGGGTKLTVL

Alternatively, a heavy chain variable domain represented by SEQ ID NO:112 can be paired with a light chain variable domain represented by SEQ ID NO:113 to form an antigen-binding site that can bind to NKG2D, as illustrated in U.S. Pat. No. 7,879,985.

SEQ ID NO: 112
QVHLQESGPGLVKPSETLSLTCTVSDDSISSYYWSWIRQPPGKGLEWIGH
ISYSGSANYNPSLKSRVTISVDTSKNQFSLKLSSVTAADTAVYYCANWDD
AFNIWGQGTMVTVSS
SEQ ID NO: 113
EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPWTFG
QGTKVEIK

(B) BCMA-Binding Site

The present disclosure provides a BCMA-binding site, in which the heavy chain variable domain and the light chain variable domain. In some embodiments, the BCMA-binding site is linked to the antibody Fc domain or the portion thereof sufficient to bind CD16, or the antigen-binding site that binds CD16 of the proteins disclosed herein via a hinge. The proteins disclosed herein can provide monovalent or bivalent engagement of BCMA, and have one or two BCMA-binding sites. In some embodiments, proteins disclosed herein have two BCMA-binding sites, each is linked to the antibody Fc domain or the portion thereof sufficient to bind CD16, or the antigen-binding site that binds CD16 of the proteins disclosed herein via a hinge.

Table 2 lists peptide sequences of heavy chain variable domains and light chain variable domains that, in combination, can bind to BCMA.

TABLE 2
Heavy chain variable domains and light chain variable
domains of the NKG2D-binding site

| | Heavy chain | Light chain |
|---|---|---|
| | variable domain | variable domain |
| Clones | peptide sequence | peptide sequence |
| 1 | QVQLVQSGAEVKKPGASVKVSCK | DIVMTQTPLSLSVTPGEPAS |
| (US14/776,649) | ASGYSFPDYYINWVRQAPGQGLE | ISCKSSQSLVHSNGNTYLHW |
| | WMGWIYFASGNSEYNQKFTGRVT | YLQKPGQSPQLLIYKVSNRF |
| | MTRDTSSSTAYMELSSLRSEDTA | SGVPDRFSGSGSGADFTLKI |
| | VYFCASLYDYDWYFDVWGQGTMV | SRVEAEDVGVYYCAETSHVP |
| | TVSS | WTFGQGTKLEIK (SEQ ID |
| | (SEQ ID NO: 114) | NO: 118) |
| | CDR1 (SEQ ID NO: 115) - | or |
| | DYYIN | DIVMTQTPLSLSVTPGQPAS |
| | CDR2 (SEQ ID NO: 116) - | ISCKSSQSLVHSNGNTYLHW |
| | WIYFASGNSEYNQKFTG | YLQKPGQSPQLLIYKVSNRF |
| | CDR3 (SEQ ID NO: 117) - | SGVPDRFSGSGSGTDFTLKI |
| | LYDYDWYFDV | SRVEAEDVGIYYCSQSSIYP |
| | | WTFGQGTKLEIK |
| | | (SEQ ID NO: 119) |
| | | CDR1 (SEQ ID NO: |
| | | 120) - |
| | | KSSQSLVHSNGNTYLH |
| | | CDR2 (SEQ ID NO: |
| | | 121) - KVSNRFS |
| | | CDR3 - AETSHVPWT |
| | | (SEQ ID NO: 122) or |
| | | SQSSIYPWT (SEQ ID |
| | | NO: 123) |
| 2 | QIQLVQSGPELKKPGETVKISCK | DIVLTQSPPSLAMSLGKRAT |
| (PCT/US15/64269) | ASGYTFTDYSINWVKRAPGKGLK | ISCRASESVTILGSHLIHWY |
| | WMGWINTETREPAYAYDFRGRFA | QQKPGQPPTLLIQLASNVQT |
| | FSLETSASTAYLQINNLKYEDTA | GVPARFSGSGSRTDFTLTID |
| | TYFCALDYSYAMDYWGQGTSVTV | PVEEDDVAVYYCLQSRTIPR |
| | SS | TFGGGTKLEIK |
| | (SEQ ID NO: 124) | (SEQ ID NO: 128) |
| | CDR1 (SEQ ID NO: 125) - | CDR1 (SEQ ID NO: |
| | DYSIN | 129) - |
| | CDR2 (SEQ ID NO: 126) - | RASESVTILGSHLIH |
| | WINTETREPAYAYDFR | CDR2 (SEQ ID NO: |
| | CDR3 (SEQ ID NO: 127) - | 130) - LASNVQT |
| | DYSYAMDY | CDR3 (SEQ ID NO: |
| | | 131) - LQSRTIPRT |
| 3 | QVQLVQSGAEVKKPGSSVKVSCK | DIQMTQSPSSLSASVGDRVT |
| (US14/122,391) | ASGGTFSNYWMHWVRQAPGQGLE | ITCSASQDISNYLNWYQQKP |
| | WMGATYRGHSDTYYNQKFKGRVT | GKAPKLLIYYTSNLHSGVPS |
| | ITADKSTSTAYMELSSLRSEDTA | RFSGSGSGTDFTLTISSLQP |
| | VYYCARGAIYNGYDVLDNWGQGT | EDFATYYCQQYRKLPWTFGQ |
| | LVTVSS | GTKLEIKR |
| | (SEQ ID NO: 132) | (SEQ ID NO: 136) |
| | CDR1 (SEQ ID NO: 133) - | CDR1 (SEQ ID NO: |
| | NYWMH | 137) - SASQDISNYLN |
| | CDR2 (SEQ ID NO: 134) - | CDR2 (SEQ ID NO: |
| | ATYRGHSDTYYNQKFKG | 138) - YTSNLHS |
| | CDR3 (SEQ ID NO: 135) - | CDR3 (SEQ ID NO: |
| | GAIYNGYDVLDN | 139) - QQYRKLPWT |
| 4 | QLQLQESGPGLVKPSETLSLTCT | SYVLTQPPSVSVAPGQTARI |
| (US20170051068) | VSGGSISSSSYFWGWIRQPPGKG | TCGGNNIGSKSVHWYQQPPG |
| | LEWIGSIYYSGITYYNPSLKSRV | QAPVVVVYDDSDRPSGIPER |
| | TISVDTSKNQFSLKLSSVTAADT | FSGSNSGNTA |
| | AVYYCARHDGATAGLFDYWGQGT | TLTISRVEAGDEAVYYCQVW |
| | LVTVSS (SEQ ID NO: 140) | DSSSDHVVFGGGTKLTVL |
| | CDR1: SSSYFWG (SEQ ID | (SEQ ID NO: 144) |
| | NO: 141) | CDR1: GGNNIGSKSVH |
| | CDR2: SIYYSGITYYNPSLKS | (SEQ ID NO: 145) |
| | (SEQ ID NO: 142) | CDR2: DDSDRPS (SEQ |
| | CDR3: HDGATAGLFDY (SEQ | ID NO: 146) |
| | ID NO: 143) | CDR3: QVWDSSSDHVV |
| | | (SEQ ID NO: 147) |
| 5 | EVQLLESGGGLVQPGGSLRLSCA | EIVLTQSPGTLSLSPGERAT |
| (Mab42 | ASGFTFSDNAMGWVRQAPGKGLE | LSCRASQSVSDEYLSWYQQK |
| (WO2017021450)) | WVSAISGPGSSTYYADSVKGRFT | PGQAPRLLIHSASTRATGIP |
| | ISRDNSKNTLYLQMNSLRAEDTA | DRFSGSGSGTDFTLAISRLE |
| | VYYCAKVLGWFDYWGQGTLVTVS | PEDFAVYYCQQYGYPPDFTF |
| | S (SEQ ID NO: 148) | GQGTKVEIK (SEQ ID |
| | CDR1: DNAMG (SEQ ID | NO: 152) |
| | NO: 149) | CDR1: RASQSVSDEYLSW |
| | CDR2: AISGPGSSTYYADSVKG | (SEQ ID NO: 153) |
| | (SEQ ID NO: 150) | CDR2: HSASTRAT (SEQ |
| | CDR3: VLGWFDY (SEQ ID | ID NO: 154) |
| | NO: 151) | CDR3: QQYGYPPDFT |
| | | (SEQ ID NO: 155) |

Alternatively, a BCMA-binding domain can include a heavy chain variable domain and light chain variable domain as listed below in 83A10 and MAB42.

83A10 Heavy Chain Variable Domain (SEQ ID NO:157):

```
EVQLLESGGGLVQPGGSLRLSCAASGFTFSSYAMSWVRQAPGKGLEWVSA
                                                            CDR1
ISGSGGSTYYADSVKGRFTISRDNSKNTLYLQMNSLRAEDTAVYYCAKVL
    CDR2
GWFDYWGQGTLVTVSS
CDR3
83A10 light chain variable domain
(SEQ ID NO: 158):
EIVLTQSPGTLSLSPGERATLSCRASQSVSSSYLAWYQQKPGQAPRLLIY
                                                CDR1
GASSRATGIPDRFSGSGSGTDFTLTISRLEPEDFAVYYCQQYGYPPDFTF
  CDR2 CDR3
GQGTKVEIK
```

Alternatively, novel antigen-binding sites that can bind to BCMA can be

identified by screening for binding to the amino acid sequence defined by SEQ ID NO:156.

SEQ ID NO: 156
MLQMAGQCSQNEYFDSLLHACIPCQLRCSSNTPPLTCQRYCNASVTNSVK
GTNAILWTCLGLSLIISLAVFVLMFLLRKINSEPLKDEFKNTGSGLLGMA
NIDLEKSRTGDEIILPRGLEYTVEECTCEDCIKSKPKVDSDHCFPLPAME
EGATILVTTKTNDYCKSLPAALSATEIEKSISAR

Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al, Nature, 406 (6793):267-273 (2000)). Based on the known domains, mutations can be selected to enhance or reduce the binding affinity to CD16, such as by using phage-displayed libraries or yeast surface-displayed cDNA libraries, or can be designed based on the known three-dimensional structure of the interaction.

The assembly of heterodimeric antibody heavy chains can be accomplished by expressing two different antibody heavy chain sequences in the same cell, which may lead to the assembly of homodimers of each antibody heavy chain as well as assembly of heterodimers. Promoting the preferential assembly of heterodimers can be accomplished by incorporating different mutations in the CH3 domain of each antibody heavy chain constant region as shown in U.S. Ser. No. 13/494,870, U.S. Ser. No. 16/028,850, U.S. Ser. No. 11/533,709, U.S. Ser. No. 12/875,015, U.S. Ser. No. 13/289,934, U.S. Ser. No. 14/773,418, U.S. Ser. No. 12/811,207, U.S. Ser. No. 13/866,756, U.S. Ser. No. 14/647,480, and U.S. Ser. No. 14/830,336. For example, mutations can be made in the CH3 domain based on human IgG1 and incorporating distinct pairs of amino acid substitutions within a first polypeptide and a second polypeptide that allow these two chains to selectively heterodimerize with each other. The positions of amino acid substitutions illustrated below are all numbered according to the EU index as in Kabat.

In one scenario, an amino acid substitution in the first polypeptide replaces the original amino acid with a larger amino acid, selected from arginine (R), phenylalanine (F), tyrosine (Y) or tryptophan (W), and at least one amino acid substitution in the second polypeptide replaces the original amino acid(s) with a smaller amino acid(s), chosen from alanine (A), serine (S), threonine (T), or valine (V), such that the larger amino acid substitution (a protuberance) fits into the surface of the smaller amino acid substitutions (a cavity). For example, one polypeptide can incorporate a T366W substitution, and the other can incorporate three substitutions including T366S, L368A, and Y407V.

An antibody heavy chain variable domain of the invention can optionally be coupled to an amino acid sequence at least 90% identical to an antibody constant region, such as an IgG constant region including hinge, CH2 and CH3 domains with or without CH1 domain. In some embodiments, the amino acid sequence of the constant region is at least 90% identical to a human antibody constant region, such as an human IgG1 constant region, an IgG2 constant region, IgG3 constant region, or IgG4 constant region. In some other embodiments, the amino acid sequence of the constant region is at least 90% identical to an antibody constant region from another mammal, such as rabbit, dog, cat, mouse, or horse. One or more mutations can be incorporated into the constant region as compared to human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y407I, Y407V, K409F, K409W, K409D, T411D, T411E, K439D, and K439E.

In certain embodiments, mutations that can be incorporated into the CH1 of a human IgG1 constant region may be at amino acid V125, F126, P127, T135, T139, A140, F170, P171, and/or V173. In certain embodiments, mutations that can be incorporated into the Cκ of a human IgG1 constant region may be at amino acid E123, F116, S176, V163, S174, and/or T164.

Amino acid substitutions could be selected from the following sets of substitutions shown in Table 3.

TABLE 3
Exemplary amino acid substitutions

| Set | First Polypeptide | Second Polypeptide |
|---|---|---|
| Set 1 | S364E/F405A | Y349K/T394F |
| Set 2 | S364H/D401K | Y349T/T411E |
| Set 3 | S364H/T394F | Y349T/F405A |
| Set 4 | S364E/T394F | Y349K/F405A |
| Set 5 | S364E/T411E | Y349K/D401K |
| Set 6 | S364D/T394F | Y349K/F405A |
| Set 7 | S364H/F405A | Y349T/T394F |
| Set 8 | S364K/E357Q | L368D/K370S |
| Set 9 | L368D/K370S | S364K |
| Set 10 | L368E/K370S | S364K |
| Set 11 | K360E/Q362E | D401K |
| Set 12 | L368D/K370S | S364K/E357L |
| Set 13 | K370S | S364K/E357Q |
| Set 14 | F405L | K409R |
| Set 15 | K409R | F405L |

Alternatively, amino acid substitutions could be selected from the following sets of substitutions shown in Table 4.

TABLE 4
Exemplary amino acid substitutions

| Set | First Polypeptide | Second Polypeptide |
|---|---|---|
| Set 1 | K409W | D399V/F405T |
| Set 2 | Y349S | E357W |
| Set 3 | K360E | Q347R |
| Set 4 | K360E/K409W | Q347R/D399V/F405T |
| Set 5 | Q347E/K360E/K409W | Q347R/D399V/F405T |
| Set 6 | Y349S/K409W | E357W/D399V/F405T |

Alternatively, amino acid substitutions could be selected from the following set of substitutions shown in Table 5.

TABLE 5
Exemplary amino acid substitutions.

| Set | First Polypeptide | Second Polypeptide |
|---|---|---|
| Set 1 | T366K/L351K | L351D/L368E |
| Set 2 | T366K/L351K | L351D/Y349E |
| Set 3 | T366K/L351K | L351D/Y349D |
| Set 4 | T366K/L351K | L351D/Y349E/L368E |
| Set 5 | T366K/L351K | L351D/Y349D/L368E |
| Set 6 | E356K/D399K | K392D/K409D |

Alternatively, at least one amino acid substitution in each polypeptide chain could be selected from Table 6.

TABLE 6
Exemplary amino acid substitutions

| First Polypeptide | Second Polypeptide |
|---|---|
| L351Y, D399R, D399K, | T366V, T366I, T366L, T366M, |
| S400K, S400R, Y407A, | N390D, N390E, K392L, K392M, |
| Y407I, Y407V | K392V, K392F |
| | K392D, K392E, K409F, K409W, |
| | T411D and T411E |

Alternatively, at least one amino acid substitutions could be selected from the following set of substitutions in Table 7, where the position(s) indicated in the First Polypeptide column is replaced by any known negatively-charged amino acid, and the position(s) indicated in the Second Polypeptide Column is replaced by any known positively-charged amino acid.

TABLE 7
Exemplary amino acid substitutions

| First Polypeptide | Second Polypeptide |
|---|---|
| K392, K370, K409, or K439 | D399, E356, or E357 |

Alternatively, at least one amino acid substitutions could be selected from the following set of in Table 8, where the position(s) indicated in the First Polypeptide column is replaced by any known positively-charged amino acid, and the position(s) indicated in the Second Polypeptide Column is replaced by any known negatively-charged amino acid.

TABLE 8
Exemplary amino acid substitutions

| First Polypeptide | Second Polypeptide |
|---|---|
| D399, E356, or E357 | K409, K439, K370, or K392 |

Alternatively, amino acid substitutions could be selected from the following set in Table 9.

TABLE 9
Exemplary amino acid substitutions

| First Polypeptide | Second Polypeptide |
|---|---|
| T350V, L351Y, | T350V, T366L, |
| F405A, and Y407V | K392L, and T394W |

Alternatively, or in addition, the structural stability of a hetero-multimeric protein may be increased by introducing S354C on either of the first or second polypeptide chain, and Y349C in the opposing polypeptide chain, which forms an artificial disulfide bridge within the interface of the two polypeptides.

In some embodiments, the amino acid sequence of one polypeptide chain of the antibody constant region differs from the amino acid sequence of an IgG1 constant region at position T366, and wherein the amino acid sequence of the other polypeptide chain of the antibody constant region differs from the amino acid sequence of an IgG1 constant region at one or more positions of T366, L368 and Y407.

In some embodiments, the amino acid sequence of one polypeptide chain of the antibody constant region differs from the amino acid sequence of an IgG1 constant region at one or more positions of T366, L368 and Y407, and wherein the amino acid sequence of the other polypeptide chain of the antibody constant region differs from the amino acid sequence of an IgG1 constant region at position T366.

In some embodiments, the amino acid sequence of one polypeptide chain of the antibody constant region differs from the amino acid sequence of an IgG1 constant region at one or more positions of E357, K360, Q362, S364, L368, K370, T394, D401, F405, and T411 and wherein the amino acid sequence of the other polypeptide chain of the antibody constant region differs from the amino acid sequence of an IgG1 constant region at one or more positions of Y349, E357, S364, L368, K370, T394, D401, F405 and T411.

In some embodiments, the amino acid sequence of one polypeptide chain of the antibody constant region differs from the amino acid sequence of an IgG1 constant region at one or more positions of Y349, E357, S364, L368, K370, T394, D401, F405 and T411 and wherein the amino acid sequence of the other polypeptide chain of the antibody constant region differs from the amino acid sequence of an IgG1 constant region at one or more positions of E357, K360, Q362, S364, L368, K370, T394, D401, F405, and T411.

In some embodiments, the amino acid sequence of one polypeptide chain of the antibody constant region differs from the amino acid sequence of an IgG1 constant region at one or more positions of L351, D399, S400 and Y407 and wherein the amino acid sequence of the other polypeptide chain of the antibody constant region differs from the amino acid sequence of an IgG1 constant region at one or more positions of T366, N390, K392, K409 and T411.

In some embodiments, the amino acid sequence of one polypeptide chain of the antibody constant region differs from the amino acid sequence of an IgG1 constant region at one or more positions of T366, N390, K392, K409 and T411 and wherein the amino acid sequence of the other polypeptide chain of the antibody constant region differs from the amino acid sequence of an IgG1 constant region at one or more positions of L351, D399, S400 and Y407.

In some embodiments, the amino acid sequence of one polypeptide chain of the antibody constant region differs from the amino acid sequence of an IgG1 constant region at one or more positions of Q347, Y349, K360, and K409, and wherein the amino acid sequence of the other polypeptide chain of the antibody constant region differs from the amino acid sequence of an IgG1 constant region at one or more positions of Q347, E357, D399 and F405.

In some embodiments, the amino acid sequence of one polypeptide chain of the antibody constant region differs from the amino acid sequence of an IgG1 constant region at one or more positions of Q347, E357, D399 and F405, and wherein the amino acid sequence of the other polypeptide chain of the antibody constant region differs from the amino acid sequence of an IgG1 constant region at one or more positions of Y349, K360, Q347 and K409.

In some embodiments, the amino acid sequence of one polypeptide chain of the antibody constant region differs from the amino acid sequence of an IgG1 constant region at one or more positions of K370, K392, K409 and K439, and wherein the amino acid sequence of the other polypeptide chain of the antibody constant region differs from the amino acid sequence of an IgG1 constant region at one or more positions of D356, E357 and D399.

In some embodiments, the amino acid sequence of one polypeptide chain of the antibody constant region differs from the amino acid sequence of an IgG1 constant region at one or more positions of D356, E357 and D399, and wherein the amino acid sequence of the other polypeptide chain of the antibody constant region differs from the amino acid sequence of an IgG1 constant region at one or more positions of K370, K392, K409 and K439.

In some embodiments, the amino acid sequence of one polypeptide chain of the antibody constant region differs from the amino acid sequence of an IgG1 constant region at one or more positions of L351, E356, T366 and D399, and wherein the amino acid sequence of the other polypeptide chain of the antibody constant region differs from the amino acid sequence of an IgG1 constant region at one or more positions of Y349, L351, L368, K392 and K409.

In some embodiments, the amino acid sequence of one polypeptide chain of the antibody constant region differs from the amino acid sequence of an IgG1 constant region at one or more positions of Y349, L351, L368, K392 and K409, and wherein the amino acid sequence of the other polypeptide chain of the antibody constant region differs from the amino acid sequence of an IgG1 constant region at one or more positions of L351, E356, T366 and D399.

In some embodiments, the amino acid sequence of one polypeptide chain of the antibody constant region differs from the amino acid sequence of an IgG1 constant region by an S354C substitution and wherein the amino acid sequence of the other polypeptide chain of the antibody constant region differs from the amino acid sequence of an IgG1 constant region by a Y349C substitution.

In some embodiments, the amino acid sequence of one polypeptide chain of the antibody constant region differs from the amino acid sequence of an IgG1 constant region by a Y349C substitution and wherein the amino acid sequence of the other polypeptide chain of the antibody constant region differs from the amino acid sequence of an IgG1 constant region by an S354C substitution.

In some embodiments, the amino acid sequence of one polypeptide chain of the antibody constant region differs from the amino acid sequence of an IgG1 constant region by K360E and K409W substitutions and wherein the amino acid sequence of the other polypeptide chain of the antibody constant region differs from the amino acid sequence of an IgG1 constant region by O347R, D399V and F405T substitutions.

In some embodiments, the amino acid sequence of one polypeptide chain of the antibody constant region differs from the amino acid sequence of an IgG1 constant region by O347R, D399V and F405T substitutions and wherein the amino acid sequence of the other polypeptide chain of the antibody constant region differs from the amino acid sequence of an IgG1 constant region by K360E and K409W substitutions.

In some embodiments, the amino acid sequence of one polypeptide chain of the antibody constant region differs from the amino acid sequence of an IgG1 constant region by a T366W substitutions and wherein the amino acid sequence of the other polypeptide chain of the antibody constant region differs from the amino acid sequence of an IgG1 constant region by T366S, T368A, and Y407V substitutions.

In some embodiments, the amino acid sequence of one polypeptide chain of the antibody constant region differs from the amino acid sequence of an IgG1 constant region by T366S, T368A, and Y407V substitutions and wherein the amino acid sequence of the other polypeptide chain of the antibody constant region differs from the amino acid sequence of an IgG1 constant region by a T366W substitution.

In some embodiments, the amino acid sequence of one polypeptide chain of the antibody constant region differs from the amino acid sequence of an IgG1 constant region by T350V, L351Y, F405A, and Y407V substitutions and wherein the amino acid sequence of the other polypeptide chain of the antibody constant region differs from the amino acid sequence of an IgG1 constant region by T350V, T366L, K392L, and T394W substitutions.

In some embodiments, the amino acid sequence of one polypeptide chain of the antibody constant region differs from the amino acid sequence of an IgG1 constant region by T350V, T366L, K392L, and T394W substitutions and wherein the amino acid sequence of the other polypeptide chain of the antibody constant region differs from the amino acid sequence of an IgG1 constant region by T350V, L351Y, F405A, and Y407V substitutions.

The multi-specific proteins described above can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the immunoglobulin light chain can be cloned into a third expression vector; and the first, second, and third expression vectors can be stably transfected together into host cells to produce the multimeric proteins.

To achieve the highest yield of the multi-specific protein, different ratios of the first, second, and third expression vector can be explored to determine the optimal ratio for transfection into the host cells. After transfection, single clones can be isolated for cell bank generation using methods known in the art, such as limited dilution, ELISA, FACS, microscopy, or Clonepix.

Clones can be cultured under conditions suitable for bio-reactor scale-up and maintained expression of the multi-specific protein. The multispecific proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

(ii) Exemplary Multi-Specific Binding Proteins

In certain embodiments, the multi-specific protein comprises a first antigen-binding site that binds NKG2D. In certain embodiments, the first antigen-binding site comprises an antibody fragment that binds NKG2D. In certain embodiments, the first antigen-binding site comprises a single-chain variable fragment (scFv) that binds NKG2D.

In certain embodiments, the first antigen-binding site that binds NKG2D comprises
(a) a heavy chain variable domain comprising complementarity-determining region 1 (CDR1) that comprises the amino acid sequence of SEQ ID NO:190, complementarity-determining region 2 (CDR2) that comprises the amino acid sequence of SEQ ID NO:96, and complementarity-determining region 3 (CDR3) that comprised the amino acid sequence of SEQ ID NO:191; and
(b) a light chain variable domain comprising CDR1 that comprises the amino acid sequence of SEQ ID NO:99, CDR2 that comprises the amino acid sequence of SEQ ID NO:100, and CDR3 that comprises the amino acid sequences of SEQ ID NO:101.

In certain embodiments, the multi-specific protein comprises a first antigen-binding site that binds NKG2D, wherein the first antigen-binding site that binds NKG2D comprises
(a) a heavy chain variable domain at least 90% identical to SEQ ID NO:94 and a light chain variable domain at least 90% identical to SEQ ID NO:98;
(b) a heavy chain variable domain at least 95% identical to SEQ ID NO:94 and a light chain variable domain at least 95% identical to SEQ ID NO:98; or
(c) a heavy chain variable domain identical to SEQ ID NO:94 and a light chain variable domain identical to SEQ ID NO:98.

In certain embodiments, the multi-specific protein comprises the second antigen-binding site that binds BCMA, wherein the second antigen-binding site that binds BCMA comprises:
(a) a heavy chain variable domain comprising CDR1 that comprises the amino acid sequence of SEQ ID NO:149, CDR2 that comprises the amino acid sequence of SEQ ID NO:150, and CDR3 that comprises the amino acid sequence of SEQ ID NO:151, and
(b) a light chain variable domain comprising CDR1 that comprises the amino acid sequence of SEQ ID NO:153, CDR2 that comprises the amino acid sequence of SEQ ID NO:154, and CDR3 that comprises the amino acid sequence of SEQ ID NO:155.

In certain embodiments, the multi-specific protein comprises the second antigen-binding site that binds BCMA, wherein the second antigen-binding site that binds BCMA comprises:
(a) a heavy chain variable domain at least 90% identical to SEQ ID NO:148 and a light chain variable domain at least 90% identical to SEQ ID NO:152;
(b) a heavy chain variable domain at least 95% identical to SEQ ID NO:148 and a light chain variable domain at least 95% identical to SEQ ID NO:152; or
(c) a heavy chain variable domain identical to SEQ ID NO:148 and a light chain variable domain identical to SEQ ID NO:152.

In certain embodiments, the multi-specific protein comprises an additional antigen-binding site that binds BCMA, wherein the additional antigen-binding site that binds BCMA comprises:
(a) a heavy chain variable domain comprising CDR1 that comprises the amino acid sequence of SEQ ID NO:149, CDR2 that comprises the amino acid sequence of SEQ ID NO:150, and CDR3 that comprises the amino acid sequence of SEQ ID NO:151, and
(b) a light chain variable domain comprising CDR1 that comprises the amino acid sequence of SEQ ID NO:153, CDR2 that comprises the amino acid sequence of SEQ ID NO:154, and CDR3 that comprises the amino acid sequence of SEQ ID NO:155.

In certain embodiments, the multi-specific protein comprises an additional antigen-binding site that binds BCMA, wherein the additional antigen-binding site that binds BCMA comprises:
(a) a heavy chain variable domain at least 90% identical to SEQ ID NO:148 and a light chain variable domain at least 90% identical to SEQ ID NO:152;
(b) a heavy chain variable domain at least 95% identical to SEQ ID NO:148 and a light chain variable domain at least 95% identical to SEQ ID NO:152; or
(c) a heavy chain variable domain identical to SEQ ID NO:148 and a light chain variable domain identical to SEQ ID NO:152.

In certain embodiments, the protein further comprises an antibody Fc domain.

In certain embodiments, the antibody Fc domain comprises hinge and CH2 domains of a human IgG1 antibody. In certain embodiments, the Fc domain comprises an amino acid sequence at least 90% identical to amino acids 234-332 of a human IgG1 antibody.

In certain embodiments, the protein comprises a polypeptide comprising the amino acid sequence of SEQ ID NO:161. In certain embodiments, the protein comprises a polypeptide comprising an amino acid sequence at least 90%, at least 95% or at least 99% identical to the amino acid sequence of SEQ ID NO:162. In certain embodiments, the protein comprises a polypeptide comprising the amino acid sequence of SEQ ID NO:162.

In certain embodiments, the protein comprises (i) a polypeptide comprising the amino acid sequence of SEQ ID NO:162; (ii) a polypeptide comprising the amino acid sequence of SEQ ID NO:163; and (iii) a polypeptide comprising the amino acid sequence of SEQ ID NO:165. In certain embodiments, the protein comprises (i) a polypeptide comprising the amino acid sequence of SEQ ID NO:162; (ii) a polypeptide comprising the amino acid sequence of SEQ ID NO:163; and (iii) two polypeptides each comprising the amino acid sequence of SEQ ID NO:165. In certain embodiments, the polypeptides are linked as represented in FIG. 15.

In certain embodiments, the polypeptide comprising the amino acid sequence of SEQ ID NO:162 is linked to the polypeptide comprising the amino acid sequence of SEQ ID NO:163 via heterodimerization and at least one disulfide bond; wherein the polypeptide comprising the amino acid sequence of SEQ ID NO:165 is linked to the polypeptide comprising the amino acid sequence of SEQ ID NO:162 via a disulfide bond; and wherein the other polypeptide comprising the amino acid sequence of SEQ ID NO:165 is linked to the polypeptide comprising the amino acid sequence of SEQ ID NO:163 via a disulfide bond.

In certain embodiments, the multi-specific binding protein is Protein 1 (illustrated in FIG. 1A) as used in multiple examples. Protein 1 comprises (i) a polypeptide comprising the amino acid sequence of SEQ ID NO:162; (ii) a polypeptide comprising the amino acid sequence of SEQ ID NO:163; and (iii) two polypeptides each comprising the amino acid sequence of SEQ ID NO:165. The polypeptides in Protein 1 are linked as represented in FIG. 15.

In certain embodiments, a multi-specific binding protein described herein further comprises an additional antigen-binding site that binds BCMA. In certain embodiments, the second antigen-binding site of a protein described in the present disclosure is an Fab fragment that binds BCMA. In certain embodiments, the second and the additional antigen-binding site of a multi-specific binding protein described herein are Fab fragments that bind BCMA.

In certain embodiments, the second and the additional antigen-binding site of a multi-specific binding protein described herein are scFvs that bind BCMA. In certain embodiments, the heavy chain variable domain of the scFv that binds NKG2D is positioned at the N-terminus or the C-terminus of the light chain variable domain of the scFv. In certain embodiments, the light chain variable domain is positioned at the N-terminus of the heavy chain variable domain of the scFv that binds NKG2D.

In certain embodiments, the scFv that binds to NKG2D is linked to the antibody Fc domain or a portion thereof sufficient to bind CD16, or a third antigen-binding site that binds CD16. In certain embodiments, the scFv that binds to NKG2D is linked to the antibody Fc domain or a portion thereof sufficient to bind CD16, or a third antigen-binding site that binds CD16 via a hinge comprising Ala-Ser. In certain embodiments, the scFv that binds to NKG2D is linked to the C-terminus of the antibody Fc domain or a portion thereof sufficient to bind CD16, or a third antigen-binding site that binds CD16 via a flexible linker comprising the amino acid sequence of SEQ ID NO:168. In certain embodiments, the flexible linker linking the C-terminus of the Fc domain to the N-terminus of the VL domain of the scFv that binds NKG2D (e.g., SEQ ID NO:98) has the amino acid sequence of SEQ ID NO:168. In certain embodiments, the C-terminus of the antibody Fc domain is linked to the N-terminus of the light chain variable domain of the scFv that binds NKG2D.

In certain embodiments, within the scFv that binds NKG2D, a disulfide bridge is formed between the heavy chain variable domain of the scFv and the light chain variable domain of the scFv. In certain embodiments, the disulfide bridge is formed between C44 from the heavy chain variable domain and C100 from the light chain variable domain.

In certain embodiments, within the scFv that binds NKG2D, the heavy chain variable domain is linked to the light chain variable domain via a flexible linker. In certain embodiments, the flexible linker comprises (GlyGlyGlyGlySer)ₙ ((G4S)ₙ; SEQ ID NO:198), wherein n is an integer between 1-10. In certain embodiments, the flexible linker comprises the amino acid sequence of SEQ ID NO:167.

In certain embodiments, the second and the additional antigen-binding site scFvs are linked to the antibody Fc domain or a portion thereof sufficient to bind CD16, or the third antigen-binding site that binds CD16, via a hinge comprising Ala-Ser. In certain embodiments, the second and the additional antigen-binding site scFvs are linked to the antibody Fc domain via a hinge comprising Ala-Ser.

In certain embodiments, a disulfide bridge is formed between the heavy chain variable domain and the light chain variable domain of the second antigen-binding site and/or the additional antigen-binding site. In certain embodiments, the disulfide bridge is formed between C44 from the heavy chain variable domain and C100 from the light chain variable domain.

In certain embodiments, the scFv that binds NKG2D comprises a light chain variable domain positioned at the N-terminus of a heavy chain variable domain, wherein the light chain variable domain is linked to the heavy chain variable domain of the scFv via a flexible linker comprising the amino acid sequence of SEQ ID NO:167, and scFv that binds NKG2D is linked to the antibody Fc domain via a hinge comprising Ala-Ser.

In certain embodiments, a multi-specific binding protein described herein comprising a first antigen-binding site comprising an scFv that binds NKG2D, comprises:
(a) a heavy chain variable domain comprising complementarity-determining region 1 (CDR1) that comprises the amino acid sequence of SEQ ID NO:190, complementarity-determining region 2 (CDR2) that comprises the amino acid sequence of SEQ ID NO:96, and complementarity-determining region 3 (CDR3) that comprised the amino acid sequence of SEQ ID NO:191; and a light chain variable domain comprising CDR1 that comprises the amino acid sequence of SEQ ID NO:99, CDR2 that comprises the amino acid sequence of SEQ ID NO:100, and CDR3 that comprises the amino acid sequences of SEQ ID NO:101.
(b) a heavy chain variable domain comprising CDR1 that comprises the amino acid sequence of SEQ ID NO:190, CDR2 that comprises the amino acid sequence of SEQ ID NO:96, and CDR3 that comprises the amino acid sequence of SEQ ID NO:193; and a light chain variable domain comprising CDR1 that comprises the amino acid sequence of SEQ ID NO:99, CDR2 that comprises the amino acid sequence of SEQ ID NO:100, and CDR3 that comprises the amino acid sequences of SEQ ID NO:101;
(c) a heavy chain variable domain comprising CDR1 that comprises the amino acid sequence of SEQ ID NO:95, CDR2 that comprises the amino acid sequence of SEQ ID NO:96, and CDR3 that comprises the amino acid sequence of SEQ ID NO:97; and a light chain variable domain comprising CDR1 that comprises the amino acid sequence of SEQ ID NO:99, CDR2 that comprises the amino acid sequence of SEQ ID NO:100, and CDR3 that comprises the amino acid sequences of SEQ ID NO:101;
(d) a heavy chain variable domain comprising CDR1 that comprises the amino acid sequence of SEQ ID NO:188, CDR2 that comprises the amino acid sequence of SEQ ID NO:88, and CDR3 that comprises the amino acid sequence of SEQ ID NO:189; and a light chain variable domain comprising CDR1 that comprises the amino acid sequence of SEQ ID NO:91, CDR2 that comprises the amino acid sequence of SEQ ID NO:92, and CDR3 that comprises the amino acid sequences of SEQ ID NO:93;
(e) a heavy chain variable domain comprising CDR1 that comprises the amino acid sequence of SEQ ID NO:185, CDR2 that comprises the amino acid sequence of SEQ ID NO:104, and CDR3 that comprises the amino acid sequence of SEQ ID NO:192; and a light chain variable domain comprising CDR1 that comprises the amino acid sequence of SEQ ID NO:107, CDR2 that comprises the amino acid sequence of SEQ ID NO:108, and CDR3 that comprises the amino acid sequences of SEQ ID NO:109;
(f) a heavy chain variable domain comprising CDR1 that comprises the amino acid sequence of SEQ ID NO:185, CDR2 that comprises the amino acid sequence of SEQ ID NO:72, and CDR3 that comprises the amino acid sequence of SEQ ID NO:159; and a light chain variable domain comprising CDR1 that comprises the amino acid sequence of SEQ ID NO:75, CDR2 that comprises the amino acid sequence of SEQ ID NO:76, and CDR3 that comprises the amino acid sequences of SEQ ID NO:77;
(g) a heavy chain variable domain comprising CDR1 that comprises the amino acid sequence of SEQ ID NO:186, CDR2 that comprises the amino acid sequence of SEQ ID NO:80, and CDR3 that comprises the amino acid sequence of SEQ ID NO:187; and a light chain variable domain comprising CDR1 that comprises the amino acid sequence of SEQ ID NO:83, CDR2 that comprises the amino acid sequence of SEQ ID NO:84, and CDR3 that comprises the amino acid sequences of SEQ ID NO:85;
(h) a heavy chain variable domain comprising CDR1 that comprises the amino acid sequence of SEQ ID NO:190, CDR2 that comprises the amino acid sequence of SEQ ID NO:96, and CDR3 that comprises the amino acid sequence of SEQ ID NO:194; and a light chain variable domain comprising CDR1 that comprises the amino acid sequence of SEQ ID NO:99, CDR2 that comprises the amino acid sequence of SEQ ID NO:100, and CDR3 that comprises the amino acid sequences of SEQ ID NO:101;
(i) a heavy chain variable domain comprising CDR1 that comprises the amino acid sequence of SEQ ID NO:190, CDR2 that comprises the amino acid sequence of SEQ ID NO:96, and CDR3 that comprises the amino acid sequence of SEQ ID NO:195; and a light chain variable domain comprising CDR1 that comprises the amino acid sequence of SEQ ID NO:99, CDR2 that comprises the amino acid sequence of SEQ ID NO:100, and CDR3 that comprises the amino acid sequences of SEQ ID NO:101;
(j) a heavy chain variable domain comprising CDR1 that comprises the amino acid sequence of SEQ ID NO:190, CDR2 that comprises the amino acid sequence of SEQ ID NO:96, and CDR3 that comprises the amino acid sequence of SEQ ID NO:196; and a light chain variable domain comprising CDR1 that comprises the amino acid sequence of SEQ ID NO:99, CDR2 that comprises the amino acid sequence of SEQ ID NO:100, and CDR3 that comprises the amino acid sequences of SEQ ID NO:101;
(k) a heavy chain variable domain comprising CDR1 that comprises the amino acid sequence of SEQ ID NO:190, CDR2 that comprises the amino acid sequence of SEQ ID NO:96, and CDR3 that comprises the amino acid sequence of SEQ ID NO:197; and a light chain variable domain comprising CDR1 that comprises the amino acid sequence of SEQ ID NO:99, CDR2 that comprises the amino acid sequence of SEQ ID NO:100, and CDR3 that comprises the amino acid sequences of SEQ ID NO:101; or
(l) a heavy chain variable domain comprising CDR1 that comprises the amino acid sequence of SEQ ID NO:190, CDR2 that comprises the amino acid sequence of SEQ ID NO:96, and CDR3 that comprises the amino acid sequence of SEQ ID NO:160; and a light chain variable domain comprising CDR1 that comprises the amino acid sequence of SEQ ID NO:99, CDR2 that comprises the amino acid sequence of SEQ ID NO:100, and CDR3 that comprises the amino acid sequences of SEQ ID NO:101;

and a second and/or an additional antigen-binding site(s) that bind(s) BCMA comprise(s):
(a) a heavy chain variable domain comprising CDR1 that comprises the amino acid sequence of SEQ ID NO:149, CDR2 that comprises the amino acid sequence of SEQ ID NO:150, and CDR3 that comprises the amino acid sequence of SEQ ID NO:151; and a light chain variable domain comprising CDR1 that comprises the amino acid sequence of SEQ ID NO:153, CDR2 that comprises the amino acid sequence of SEQ ID NO:154, and CDR3 that comprises the amino acid sequences of SEQ ID NO:155;
(b) a heavy chain variable domain comprising CDR1 that comprises the amino acid sequence of SEQ ID NO:115, CDR2 that comprises the amino acid sequence of SEQ ID NO:116, and CDR3 that comprises the amino acid sequence of SEQ ID NO:117; and a light chain variable domain comprising CDR1 that comprises the amino acid sequence of SEQ ID NO:120, CDR2 that comprises the amino acid sequence of SEQ ID NO:121, and CDR3 that comprises the amino acid sequences of SEQ ID NO:123;
(c) a heavy chain variable domain comprising CDR1 that comprises the amino acid sequence of SEQ ID NO:125, CDR2 that comprises the amino acid sequence of SEQ ID NO:126, and CDR3 that comprises the amino acid sequence of SEQ ID NO:127; and a light chain variable domain comprising CDR1 that comprises the amino acid sequence of SEQ ID NO:129, CDR2 that comprises the amino acid sequence of SEQ ID NO:130, and CDR3 that comprises the amino acid sequences of SEQ ID NO:131;
(d) a heavy chain variable domain comprising CDR1 that comprises the amino acid sequence of SEQ ID NO:133, CDR2 that comprises the amino acid sequence of SEQ ID NO:134, and CDR3 that comprises the amino acid sequence of SEQ ID NO:135; and a light chain variable domain comprising CDR1 that comprises the amino acid sequence of SEQ ID NO:137, CDR2 that comprises the amino acid sequence of SEQ ID NO:138, and CDR3 that comprises the amino acid sequences of SEQ ID NO:139;
(e) a heavy chain variable domain comprising CDR1 that comprises the amino acid sequence of SEQ ID NO:141, CDR2 that comprises the amino acid sequence of SEQ ID NO:142, and CDR3 that comprises the amino acid sequence of SEQ ID NO:143; and a light chain variable domain comprising CDR1 that comprises the amino acid sequence of SEQ ID NO:145, CDR2 that comprises the amino acid sequence of SEQ ID NO:146, and CDR3 that comprises the amino acid sequences of SEQ ID NO:147; or
(f) a heavy chain variable domain comprising CDR1 that comprises the amino acid sequence of SEQ ID NO:115, CDR2 that comprises the amino acid sequence of SEQ ID NO:116, and CDR3 that comprises the amino acid sequence of SEQ ID NO:117; and a light chain variable domain comprising CDR1 that comprises the amino acid sequence of SEQ ID NO:120, CDR2 that comprises the amino acid sequence of SEQ ID NO:121, and CDR3 that comprises the amino acid sequences of SEQ ID NO:122.

In certain embodiments, a multi-specific binding protein described herein comprises the amino acid sequence of SEQ ID NO:162.

In certain embodiments, a multi-specific binding protein described herein comprises an amino acid sequence comprising SEQ ID NO:162, SEQ ID NO:163, and SEQ ID NO:165.

In certain embodiments, a multi-specific binding protein described herein comprises an amino acid sequence at least 90% identical to the amino acid sequence of SEQ ID NO:162.

In certain embodiments, a multi-specific binding protein described herein comprises an amino acid sequence at least 95% identical to the amino acid sequence of SEQ ID NO:162.

In certain embodiments, a multi-specific binding protein described herein comprises an amino acid sequence at least 99% identical to the amino acid sequence of SEQ ID NO:162.

In certain embodiments, a multi-specific binding protein described herein comprises an amino acid sequence at least 90%, at least 95%, or at least 99% identical to the amino acid sequence of SEQ ID NO:162, and further comprises SEQ ID NO:163 and SEQ ID NO:165.

In certain embodiments, a multi-specific binding protein described herein includes a first antigen-binding site that binds NKG2D which comprises an antibody fragment that binds NKG2D. In certain embodiments, a multi-specific binding protein described herein includes a first antigen-binding site that binds NKG2D which comprises a single-chain variable fragment (scFv) that binds NKG2D.

In certain embodiments, a multi-specific binding protein described herein includes a first antigen-binding site that binds NKG2D, which comprises a heavy chain variable domain at least 90% (e.g., at least 91%, 92%, 93%, 94%, 95%, 96%, 97%, 98%, or 99% identical) identical to the amino acid sequence of SEQ ID NO:94.

In certain embodiments, a multi-specific binding protein described herein includes a first antigen-binding site that binds NKG2D, which comprises a heavy chain variable domain at least 90% (e.g., at least 91%, 92%, 93%, 94%, 95%, 96%, 97%, 98%, or 99% identical) identical to SEQ ID NO:94 and a light chain variable domain at least 90% identical to SEQ ID NO:98. In certain embodiments, a multi-specific binding protein described herein includes a first antigen-binding site that binds NKG2D, which comprises a heavy chain variable domain at least 95% identical to SEQ ID NO:94 and a light chain variable domain at least 95% identical to SEQ ID NO:98. In certain embodiments, a multi-specific binding protein described herein comprises a first antigen-binding site that binds NKG2D, which comprises a heavy chain variable domain at least 98% identical to SEQ ID NO:94 and a light chain variable domain at least 98% identical to SEQ ID NO:98. In certain embodiments, a multi-specific binding protein described herein comprises a first antigen-binding site that binds NKG2D, which comprises a heavy chain variable domain at least 99% identical to SEQ ID NO:94 and a light chain variable domain at least 99% identical to SEQ ID NO:98. In certain embodiments, a multi-specific binding protein described herein includes a first antigen-binding site that binds NKG2D, which comprises a heavy chain variable domain identical to SEQ ID NO:94 and a light chain variable domain identical to SEQ ID NO:98.

In certain embodiments, a multi-specific binding protein described herein includes a first antigen-binding site that binds NKG2D, which comprises a heavy chain variable domain at least 90% (e.g., at least 91%, 92%, 93%, 94%, 95%, 96%, 97%, 98%, or 99% identical) to SEQ ID NO:169 and a light chain variable domain at least 90% identical to SEQ ID NO:98. In certain embodiments, a multi-specific binding protein described herein includes a first antigen-binding site that binds NKG2D, which comprises a heavy chain variable domain at least 95% identical to SEQ ID NO:169 and a light chain variable domain at least 95% identical to SEQ ID NO:98. In certain embodiments, a multi-specific binding protein described herein includes a first antigen-binding site that binds NKG2D, which comprises a heavy chain variable domain at least 98% identical to SEQ ID NO:169 and a light chain variable domain at least 98% identical to SEQ ID NO:98. In certain embodiments, a multi-specific binding protein described herein includes a first antigen-binding site that binds NKG2D, which comprises a heavy chain variable domain at least 99% identical to SEQ ID NO:169 and a light chain variable domain at least 99% identical to SEQ ID NO:98. In certain embodiments, a multi-specific binding protein described herein includes a first antigen-binding site that binds NKG2D, which comprises a heavy chain variable domain identical to SEQ ID NO:169 and a light chain variable domain identical to SEQ ID NO:98.

In certain embodiments, a multi-specific binding protein described herein includes a first antigen-binding site that binds NKG2D, which comprises a heavy chain variable domain at least 90% (e.g., at least 91%, 92%, 93%, 94%, 95%, 96%, 97%, 98%, or 99% identical) identical to SEQ ID NO:171 and a light chain variable domain at least 90% identical to SEQ ID NO:98. In certain embodiments, a multi-specific binding protein described herein includes a first antigen-binding site that binds NKG2D, which comprises a heavy chain variable domain at least 95% identical to SEQ ID NO:171 and a light chain variable domain at least 95% identical to SEQ ID NO:98. In certain embodiments, a multi-specific binding protein described herein includes a first antigen-binding site that binds NKG2D, which comprises a heavy chain variable domain at least 98% identical to SEQ ID NO:171 and a light chain variable domain at least 98% identical to SEQ ID NO:98. In certain embodiments, a multi-specific binding protein described herein includes a first antigen-binding site that binds NKG2D, which comprises a heavy chain variable domain at least 99% identical to SEQ ID NO:171 and a light chain variable domain at least 99% identical to SEQ ID NO:98. In certain embodiments, a multi-specific binding protein described herein includes a first antigen-binding site that binds NKG2D, which comprises a heavy chain variable domain identical to SEQ ID NO:171 and a light chain variable domain identical to SEQ ID NO:98.

In certain embodiments, a multi-specific binding protein described herein includes a first antigen-binding site that binds NKG2D, which comprises a heavy chain variable domain at least 90% (e.g., at least 91%, 92%, 93%, 94%, 95%, 96%, 97%, 98%, or 99% identical) identical to SEQ ID NO:173 and a light chain variable domain at least 90% identical to SEQ ID NO:98. In certain embodiments, a multi-specific binding protein described herein includes a first antigen-binding site that binds NKG2D, which comprises a heavy chain variable domain at least 95% identical to SEQ ID NO:173 and a light chain variable domain at least 95% identical to SEQ ID NO:98. In certain embodiments, a multi-specific binding protein described herein includes a first antigen-binding site that binds NKG2D, which comprises a heavy chain variable domain at least 98% identical to SEQ ID NO:173 and a light chain variable domain at least 98% identical to SEQ ID NO:98. In certain embodiments, a multi-specific binding protein described herein includes a first antigen-binding site that binds NKG2D, which comprises a heavy chain variable domain at least 99% identical to SEQ ID NO:173 and a light chain variable domain at least 99% identical to SEQ ID NO:98. In certain embodiments, a multi-specific binding protein described herein includes a first antigen-binding site that binds NKG2D, which comprises a heavy chain variable domain identical to SEQ ID NO:173 and a light chain variable domain identical to SEQ ID NO:98.

In certain embodiments, a multi-specific binding protein described herein includes a first antigen-binding site that binds NKG2D, which comprises a heavy chain variable domain at least 90% (e.g., at least 91%, 92%, 93%, 94%, 95%, 96%, 97%, 98%, or 99% identical) identical to SEQ ID NO:175 and a light chain variable domain at least 90% identical to SEQ ID NO:98. In certain embodiments, a multi-specific binding protein described herein includes a first antigen-binding site that binds NKG2D, which comprises a heavy chain variable domain at least 95% identical to SEQ ID NO:175 and a light chain variable domain at least 95% identical to SEQ ID NO:98. In certain embodiments, a multi-specific binding protein described herein includes a first antigen-binding site that binds NKG2D, which comprises a heavy chain variable domain at least 98% identical to SEQ ID NO:175 and a light chain variable domain at least 98% identical to SEQ ID NO:98. In certain embodiments, a multi-specific binding protein described herein includes a first antigen-binding site that binds NKG2D, which comprises a heavy chain variable domain at least 99% identical to SEQ ID NO:175 and a light chain variable domain at least 99% identical to SEQ ID NO:98. In certain embodiments, a multi-specific binding protein described herein includes a first antigen-binding site that binds NKG2D, which comprises a heavy chain variable domain identical to SEQ ID NO:175 and a light chain variable domain identical to SEQ ID NO:98.

In certain embodiments, a multi-specific binding protein described herein includes a first antigen-binding site that binds NKG2D, which comprises a heavy chain variable domain at least 90% (e.g., at least 91%, 92%, 93%, 94%, 95%, 96%, 97%, 98%, or 99% identical) identical to SEQ ID NO:177 and a light chain variable domain at least 90% identical to SEQ ID NO:98. In certain embodiments, a multi-specific binding protein described herein includes a first antigen-binding site that binds NKG2D, which comprises a heavy chain variable domain at least 95% identical to SEQ ID NO:177 and a light chain variable domain at least 95% identical to SEQ ID NO:98. In certain embodiments, a multi-specific binding protein described herein includes a first antigen-binding site that binds NKG2D, which comprises a heavy chain variable domain at least 98% identical to SEQ ID NO:177 and a light chain variable domain at least 98% identical to SEQ ID NO:98. In certain embodiments, a multi-specific binding protein described herein includes a first antigen-binding site that binds NKG2D, which comprises a heavy chain variable domain at least 99% identical to SEQ ID NO:177 and a light chain variable domain at least 99% identical to SEQ ID NO:98. In certain embodiments, a multi-specific binding protein described herein includes a first antigen-binding site that binds NKG2D, which comprises a heavy chain variable domain identical to SEQ ID NO:177 and a light chain variable domain identical to SEQ ID NO:98.

In certain embodiments, a multi-specific binding protein described herein includes a first antigen-binding site that binds NKG2D, which comprises a heavy chain variable domain at least 90% (e.g., at least 91%, 92%, 93%, 94%, 95%, 96%, 97%, 98%, or 99% identical) identical to SEQ ID NO:179 and a light chain variable domain at least 90% identical to SEQ ID NO:98. In certain embodiments, a multi-specific binding protein described herein includes a first antigen-binding site that binds NKG2D, which comprises a heavy chain variable domain at least 95% identical to SEQ ID NO:179 and a light chain variable domain at least 95% identical to SEQ ID NO:98. In certain embodiments, a multi-specific binding protein described herein includes a first antigen-binding site that binds NKG2D, which comprises a heavy chain variable domain at least 98% identical to SEQ ID NO:179 and a light chain variable domain at least 98% identical to SEQ ID NO:98. In certain embodiments, a multi-specific binding protein described herein includes a first antigen-binding site that binds NKG2D, which comprises a heavy chain variable domain at least 99% identical to SEQ ID NO:179 and a light chain variable domain at least 99% identical to SEQ ID NO:98. In certain embodiments, a multi-specific binding protein described herein includes a first antigen-binding site that binds NKG2D, which comprises a heavy chain variable domain identical to SEQ ID NO:179 and a light chain variable domain identical to SEQ ID NO:98.

In certain embodiments, a multi-specific binding protein described herein includes a second antigen-binding site that binds BCMA, which comprises a heavy chain variable domain at least 90% (e.g., at least 91%, 92%, 93%, 94%, 95%, 96%, 97%, 98%, or 99% identical) identical to SEQ ID NO:148 and a light chain variable domain at least 90% identical to SEQ ID NO:152. In certain embodiments, a multi-specific binding protein described herein includes a first antigen-binding site that binds BCMA, which comprises a heavy chain variable domain at least 95% identical to SEQ ID NO:148 and a light chain variable domain at least 95% identical to SEQ ID NO:152. In certain embodiments, a multi-specific binding protein described herein includes a first antigen-binding site that binds BCMA, which comprises a heavy chain variable domain at least 98% identical to SEQ ID NO:148 and a light chain variable domain at least 98% identical to SEQ ID NO:152. In certain embodiments, a multi-specific binding protein described herein includes a first antigen-binding site that binds BCMA, which comprises a heavy chain variable domain at least 99% identical to SEQ ID NO:148 and a light chain variable domain at least 99% identical to SEQ ID NO:152. In certain embodiments, a multi-specific binding protein described herein includes a first antigen-binding site that binds BCMA, which comprises a heavy chain variable domain identical to SEQ ID NO:148 and a light chain variable domain identical to SEQ ID NO:152.

In certain embodiments, a multi-specific binding protein described herein includes a second antigen-binding site that binds BCMA, which comprises a heavy chain variable domain at least 90% (e.g., at least 91%, 92%, 93%, 94%, 95%, 96%, 97%, 98%, or 99% identical) identical to SEQ ID NO:114 and a light chain variable domain at least 90% identical to SEQ ID NO:119. In certain embodiments, a multi-specific binding protein described herein includes a first antigen-binding site that binds BCMA, which comprises a heavy chain variable domain at least 95% identical to SEQ ID NO:114 and a light chain variable domain at least 95% identical to SEQ ID NO:119. In certain embodiments, a multi-specific binding protein described herein includes a first antigen-binding site that binds BCMA, which comprises a heavy chain variable domain at least 98% identical to SEQ ID NO:114 and a light chain variable domain at least 98% identical to SEQ ID NO:119. In certain embodiments, a multi-specific binding protein described herein includes a first antigen-binding site that binds BCMA, which comprises a heavy chain variable domain at least 99% identical to SEQ ID NO:114 and a light chain variable domain at least 99% identical to SEQ ID NO:119. In certain embodiments, a multi-specific binding protein described herein includes a first antigen-binding site that binds BCMA, which comprises a heavy chain variable domain identical to SEQ ID NO:114 and a light chain variable domain identical to SEQ ID NO:119.

In certain embodiments, a multi-specific binding protein described herein includes a second antigen-binding site that binds BCMA, which comprises a heavy chain variable domain at least 90% (e.g., at least 91%, 92%, 93%, 94%, 95%, 96%, 97%, 98%, or 99% identical) identical to SEQ ID NO:124 and a light chain variable domain at least 90% identical to SEQ ID NO:128. In certain embodiments, a multi-specific binding protein described herein includes a first antigen-binding site that binds BCMA, which comprises a heavy chain variable domain at least 95% identical to SEQ ID NO:124 and a light chain variable domain at least 95% identical to SEQ ID NO:128. In certain embodiments, a multi-specific binding protein described herein includes a first antigen-binding site that binds BCMA, which comprises a heavy chain variable domain at least 98% identical to SEQ ID NO:124 and a light chain variable domain at least 98% identical to SEQ ID NO:128. In certain embodiments, a multi-specific binding protein described herein includes a first antigen-binding site that binds BCMA, which comprises a heavy chain variable domain at least 99% identical to SEQ ID NO:124 and a light chain variable domain at least 99% identical to SEQ ID NO:128. In certain embodiments, a multi-specific binding protein described herein includes a first antigen-binding site that binds BCMA, which comprises a heavy chain variable domain identical to SEQ ID NO:124 and a light chain variable domain identical to SEQ ID NO:128.

In certain embodiments, a multi-specific binding protein described herein includes a second antigen-binding site that binds BCMA, which comprises a heavy chain variable domain at least 90% (e.g., at least 91%, 92%, 93%, 94%, 95%, 96%, 97%, 98%, or 99% identical) identical to SEQ ID NO:132 and a light chain variable domain at least 90% identical to SEQ ID NO:136. In certain embodiments, a multi-specific binding protein described herein includes a first antigen-binding site that binds BCMA, which comprises a heavy chain variable domain at least 95% identical to SEQ ID NO:132 and a light chain variable domain at least 95% identical to SEQ ID NO:136. In certain embodiments, a multi-specific binding protein described herein includes a first antigen-binding site that binds BCMA, which comprises a heavy chain variable domain at least 98% identical to SEQ ID NO:132 and a light chain variable domain at least 98% identical to SEQ ID NO:136. In certain embodiments, a multi-specific binding protein described herein includes a first antigen-binding site that binds BCMA, which comprises a heavy chain variable domain at least 99% identical to SEQ ID NO:132 and a light chain variable domain at least 99% identical to SEQ ID NO:136. In certain embodiments, a multi-specific binding protein described herein includes a first antigen-binding site that binds BCMA, which comprises a heavy chain variable domain identical to SEQ ID NO:132 and a light chain variable domain identical to SEQ ID NO:136.

In certain embodiments, a multi-specific binding protein described herein includes a second antigen-binding site that binds BCMA, which comprises a heavy chain variable domain at least 90% (e.g., at least 91%, 92%, 93%, 94%, 95%, 96%, 97%, 98%, or 99% identical) identical to SEQ ID NO:140 and a light chain variable domain at least 90% identical to SEQ ID NO:144. In certain embodiments, a multi-specific binding protein described herein includes a first antigen-binding site that binds BCMA, which comprises a heavy chain variable domain at least 95% identical to SEQ ID NO:140 and a light chain variable domain at least 95% identical to SEQ ID NO:144. In certain embodiments, a multi-specific binding protein described herein includes a first antigen-binding site that binds BCMA, which comprises a heavy chain variable domain at least 98% identical to SEQ ID NO:140 and a light chain variable domain at least 98% identical to SEQ ID NO:144. In certain embodiments, a multi-specific binding protein described herein includes a first antigen-binding site that binds BCMA, which comprises a heavy chain variable domain at least 99% identical to SEQ ID NO:140 and a light chain variable domain at least 99% identical to SEQ ID NO:144. In certain embodiments, a multi-specific binding protein described herein includes a first antigen-binding site that binds BCMA, which comprises a heavy chain variable domain identical to SEQ ID NO:140 and a light chain variable domain identical to SEQ ID NO:144.

In certain embodiments, a multi-specific binding protein described herein includes a second antigen-binding site that binds BCMA, which comprises a heavy chain variable domain at least 90% (e.g., at least 91%, 92%, 93%, 94%, 95%, 96%, 97%, 98%, or 99% identical) identical to SEQ ID NO:114 and a light chain variable domain at least 90% identical to SEQ ID NO:118. In certain embodiments, a multi-specific binding protein described herein includes a first antigen-binding site that binds BCMA, which comprises a heavy chain variable domain at least 95% identical to SEQ ID NO:114 and a light chain variable domain at least 95% identical to SEQ ID NO:118. In certain embodiments, a multi-specific binding protein described herein includes a first antigen-binding site that binds BCMA, which comprises a heavy chain variable domain at least 98% identical to SEQ ID NO:114 and a light chain variable domain at least 98% identical to SEQ ID NO:118. In certain embodiments, a multi-specific binding protein described herein includes a first antigen-binding site that binds BCMA, which comprises a heavy chain variable domain at least 99% identical to SEQ ID NO:114 and a light chain variable domain at least 99% identical to SEQ ID NO:118. In certain embodiments, a multi-specific binding protein described herein includes a first antigen-binding site that binds BCMA, which comprises a heavy chain variable domain identical to SEQ ID NO:114 and a light chain variable domain identical to SEQ ID NO:118.

In certain embodiments, the multi-specific binding protein described herein further comprises an additional antigen-binding site that binds BCMA. In certain embodiments, the additional antigen-binding site comprises the same CDR1, CDR2, and CDR3 of heavy chain variable domain and the same CDR1, CDR2, and CDR3 of light chain variable domain of the second antigen-binding site that binds BCMA. In certain embodiments, the additional antigen-binding site comprises a heavy chain variable domain at least 90% (e.g., at least 91%, 92%, 93%, 94%, 95%, 96%, 97%, 98%, or 99% identical) identical to the heavy chain variable domain of the second antigen-binding site that binds BCMA, and a light chain variable domain at least 90% (e.g., at least 91%, 92%, 93%, 94%, 95%, 96%, 97%, 98%, or 99% identical) identical to the light chain variable domain of the second antigen-binding site that binds BCMA. In certain embodiments, the additional antigen-binding site comprises a heavy chain variable domain identical to the heavy chain variable domain of the second antigen-binding site that binds BCMA, and a light chain variable domain identical to the light chain variable domain of the second antigen-binding site that binds BCMA.

In certain embodiments, a multi-specific binding protein described herein comprises an antibody Fc domain or a portion thereof sufficient to bind CD16, or a third antigen-binding site that binds CD16. In certain embodiments, a multi-specific binding protein described herein comprises an antibody Fc domain. The antibody Fc domain can bind CD16. In certain embodiments, a multi-specific binding protein described herein comprises a portion of an antibody Fc domain that retains the binding affinity of the antibody Fc domain to CD16, i.e., sufficient to bind CD16. In certain embodiments, a multi-specific binding protein described herein comprises a third antigen-binding site that binds CD16. In certain embodiments, the third antigen-binding site that binds CD16 comprises a Fab fragment. In certain embodiments, the third antigen-binding site that binds CD16 comprises a scFV.

In certain embodiments, the scFv that binds to NKG2D is linked to the antibody Fc domain or the portion thereof sufficient to bind CD16, or the third antigen-binding site that binds CD16. In certain embodiments, the scFv that binds to NKG2D is linked to the antibody Fc domain or the portion thereof sufficient to bind CD16, or the third antigen-binding site that binds CD16 via a hinge comprising Ala-Ser. In certain embodiments, the scFv that binds to NKG2D is linked to the C-terminus of the antibody Fc domain or the portion thereof sufficient to bind CD16, or the third antigen-binding site that binds CD16 via a flexible linker. In certain embodiments, the flexible linker comprises the amino acid sequence of SEQ ID NO:168. In certain embodiments, the C-terminus of the antibody Fc domain or the portion thereof sufficient to bind CD16, or the third antigen-binding site that binds CD16 is linked to the N-terminus of the light chain variable domain of the scFv that binds NKG2D. In certain embodiments, the flexible linker linking the C-terminus of the antibody Fc domain or the portion thereof sufficient to bind CD16, or the third antigen-binding site that binds CD16, to the N-terminus of the VL domain of the scFv that binds NKG2D (e.g., SEQ ID NO:98) has the amino acid sequence of SEQ ID NO:168.

In certain embodiments, a multi-specific binding protein described herein comprises an antibody Fc domain. In certain embodiments, the scFv that binds to NKG2D is linked to the antibody Fc domain. In certain embodiments, the scFv that binds to NKG2D is linked to the antibody Fc domain via a hinge comprising Ala-Ser. In certain embodiments, the scFv that binds to NKG2D is linked to the C-terminus of the antibody Fc domain via a flexible linker. In certain embodiments, the flexible linker comprises the amino acid sequence of SEQ ID NO:167 or SEQ ID NO:168. In certain embodiments, the C-terminus of the antibody Fc domain is linked to the N-terminus of the light chain variable domain of the scFv that binds NKG2D. In certain embodiments, the flexible linker linking the C-terminus of the Fc domain to the N-terminus of the VL domain of the scFv that binds NKG2D (e.g., SEQ ID NO:98) has the amino acid sequence of SEQ ID NO:167 or SEQ ID NO:168.

In certain embodiments, a multi-specific binding protein described herein includes an antibody Fc domain comprising hinge and CH2 domains of a human IgG1 antibody.

In certain embodiments, a multi-specific binding protein described herein includes an Fc domain comprising an amino acid sequence at least 90% identical to amino acids 234-332 of a human IgG1 antibody. In certain embodiments, a multi-specific binding protein described herein includes an Fc domain comprising an amino acid sequence at least 95% identical to amino acids 234-332 of a human IgG1 antibody. In certain embodiments, a multi-specific binding protein described herein includes an Fc domain comprising an amino acid sequence at least 98% identical to amino acids 234-332 of a human IgG1 antibody. In certain embodiments, a multi-specific binding protein described herein includes an Fc domain comprising amino acid sequence at least 90% identical to the Fc domain of human IgG1. In certain embodiments, a multi-specific binding protein described herein includes an Fc domain comprising amino acid sequence at least 95% identical to the Fc domain of human IgG1. In certain embodiments, a multi-specific binding protein described herein includes an Fc domain comprising amino acid sequence at least 98% identical to the Fc domain of human IgG1. In certain embodiments, a multi-specific binding protein described herein includes an Fc domain comprising amino acid sequence at least 90% identical to the Fc domain of human IgG1 and differs at one or more positions of Q347, Y349, T350, L351, S354, E356, E357, K360, Q362, S364, T366, L368, K370, N390, K392, T394, D399, S400, D401, F405, Y407, K409, T411, and K439.

In certain embodiments, a multi-specific binding protein described herein includes an Fc domain of an human IgG1 comprising Q347R, D399V, and F405T substitutions. A multi-specific binding protein described herein includes an Fc domain comprising Q347R, D399V, and F405T substitutions, linked to an scFv that bind NKG2D.

In certain embodiments, a multi-specific binding protein described herein includes an Fc domain of an human IgG1 comprising K360E and K409W substitutions.

In certain embodiments, a multi-specific binding protein described herein includes an Fc domain comprising K360E and K409W substitutions, linked to the second antigen binding site.

In certain embodiments, the first antigen-binding site binds to NKG2D with a K_{D} of 2 to 120 nM, as measured by surface plasmon resonance. In certain embodiments, a multi-specific binding protein described herein binds to NKG2D with a K_{D} of 2 to 120 nM, as measured by surface plasmon resonance.

### 5.3 Iberdomide (CC-220)

In one aspect, iberdomide (CC-220) can be used with the combination treatments described herein. In certain embodiments, iberdomide as described herein can be administered in combination with the protein that comprises an antigen-binding site that binds to an antigen on a natural killer (NK) cell as described in Section 5.1 in the methods of treating, managing, and/or preventing cancer described in Section 5.8. In certain embodiments, iberdomide as described herein can be administered in combination with the protein that comprises an antigen-binding site that binds NKG2D as described in Section 5.2 in the in the methods of treating, managing, and/or preventing cancer described in Section 5.8.

In certain embodiments, iberdomide (CAS Registry Number 1323403-33-3) has the following structure:

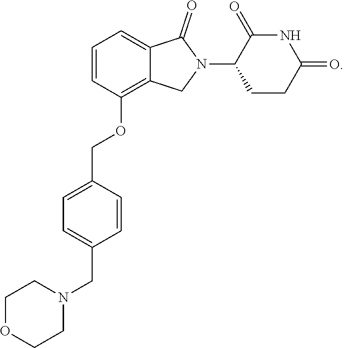 [SMILES: O=C1CC[C@H](N2Cc3c(OCc4ccc(CN5CCOCC5)cc4)cccc3C2=O)C(=O)N1]

In certain embodiments, iberdomide as described herein is referred to as (S)-3-(4-((4-(Morpholinomethyl)benzyl)oxy)-1-oxoisoindolin-2-yl)piperidine-2,6-dione, (3s)-3-[4-({4-[(Morpholin-4-Yl)methyl]phenyl}methoxy)-1-Oxo-1,3-Dihydro-2h-Isoindol-2-Yl]piperidine-2,6-Dione, (3 S)-3-[7-[[4-(morpholin-4-ylmethyl)phenyl]methoxy]-3-oxo-1H-isoindol-2-yl]piperidine-2,6-dione, (S)-3-[4-(4-Morpholin-4-yl-methyl-benzyloxy)-1-oxo-1,3-dihydro-isoindol-2-yl]-piperidine-2,6-dione, or (3S)-3-[7-[[4-(morpholin-4-ylmethyl)phenyl]methoxy]-3-oxo-1H-isoindol-2-yl]piperidine-2,6-dione. Methods of preparing iberdomide are described, e.g., in US 20110196150, the entirety of which is incorporated herein by reference.

In certain embodiments, iberdomide as described herein can be in a form of hydrochloride salt. In certain embodiments, iberdomide hydrochloride is referred to as (3S)-3-(4-{[4-(morpholin-4-ylmethyl)benzyl] oxy}-1-oxo-1,3-dihydro-2H-isoindol-2-yl)piperidine-2,6-dione hydrochloride (1:1) or 2,6-piperidinedione, 3-[1,3-dihydro-4-[[4-(4-morpholinylmethyl)phenyl]methoxy]-1-oxo-2H-isoindol-2-yl]-, (3S)-, hydrochloride (1:1).

Without being bound by any particular theory, iberdomide as described herein may function as described in the embodiments below. In certain embodiments, iberdomide as described herein and the protein as described in Section 5.1 or Section 5.2 may activate NK cells to a greater degree than either of the protein and said iberdomide alone. In certain embodiments, iberdomide as described herein and the protein as described herein may activate an immune cell other than NK cells to a greater degree than either of the protein and said iberdomide alone. In certain embodiments, iberdomide as described herein may have synergistic anti-cancer effect with the protein as described herein.

### 5.4 Avadomide (CC-122)

In one aspect, avadomide (CC-122) can be used with the combination treatments described herein. In certain embodiments, avadomide as described herein can be administered in combination with the protein that comprises an antigen-binding site that binds to an antigen on a natural killer (NK) cell as described in Section 5.1 in the methods of treating, managing, and/or preventing cancer described in Section 5.8. In certain embodiments, avadomide as described herein can be administered in combination with the protein that comprises an antigen-binding site that binds NKG2D as described in Section 5.2 in the in the methods of treating, managing, and/or preventing cancer described in Section 5.8.

In certain embodiments, avadomide (CAS Registry Number 1015474-32-4) has the following structure:

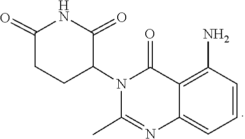 [SMILES: Cc1nc2cccc(N)c2c(=O)n1C1CCC(=O)NC1=O]

In certain embodiments, avadomide as described herein is referred to as 3-(5-amino-2-methyl-4-oxoquinazolin-3(4H)-yl)piperidine-2,6-dione, 3-(5-amino-2-methyl-4-oxo-4H-quinazolin-3-yl)-piperidine-2,6-dione, or 3-(5-amino-2-methyl-4-oxoquinazolin-3-yl)piperidine-2,6-dione.

In certain embodiments, avadomide as described herein can be in a form of hydrochloride salt. In certain embodiments, avadomide hydrochloride (CAS Registry Number 1398053-45-6) is referred to as 3-(5-amino-2-methyl-4-oxoquinazolin-3-yl)piperidine-2,6-dione hydrochloride, 2,6-piperidinedione,3-(5-amino-2-methyl-4-oxo-3(4H-quinazolinyl), hydrochloride (1:1), or (3RS)-3-(5-amino-2-methyl-4-oxoquinazolin-3(4H)-yl)piperidine-2,6-dione monohydrochloride.

Without being bound by any particular theory, avadomide as described herein may function as described in the embodiments below. In certain embodiments, avadomide as described herein and the protein as described in Section 5.1 or Section 5.2 may activate NK cells to a greater degree than either of the protein and said avadomide alone. In certain embodiments, avadomide as described herein and the protein as described herein may activate an immune cell other than NK cells to a greater degree than either of the protein and said avadomide alone. In certain embodiments, avadomide as described herein may have synergistic anti-cancer effect with the protein as described herein.

### 5.5 Lenalidomide

In one aspect, lenalidomide can be used with the combination treatments described herein. In certain embodiments, lenalidomide as described herein can be administered in combination with the protein that comprises an antigen-binding site that binds to an antigen on a natural killer (NK) cell as described in Section 5.1 in the methods of treating, managing, and/or preventing cancer described in Section 5.8. In certain embodiments, lenalidomide as described herein can be administered in combination with the protein that comprises an antigen-binding site that binds NKG2D as described in Section 5.2 in the in the methods of treating, managing, and/or preventing cancer described in Section 5.8.

In certain embodiments, lenalidomide (CAS Registry Number 191732-72-6) has the following structure:

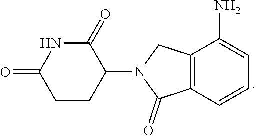 [SMILES: Nc1cccc2c1CN(C1CCC(=O)NC1=O)C2=O]

In certain embodiments, lenalidomide as described herein is referred to as 3-(7-amino-3-oxo-1H-isoindol-2-yl)piperidine-2,6-dione, 3-(4-amino-1-oxoisoindolin-2-yl)piperidine-2,6-dione, 3-(4-Amino-1-oxo-1,3-dihydro-2H-isoindol-2-yl)piperidine-2,6-dione, 3-(7-amino-3-oxo-1H-isoindol-2-yl)piperidine-2,6-dione, 3-(4-amino-1-oxo-2,3-dihydro-1H-isoindol-2-yl)piperidine-2,6-dione, 3-(4-Amino-1-oxo-1,3-dihydro-isoindol-2-yl)-piperidine-2,6-dione, 3-(4-amino-1,3-dihydro-1-oxo-2h-isoindol-2-yl)-2,6-piperidinedione, 3-(4-amino-1-oxo-1,3-dihydro-2H-isoindol-2-yl)-2,6-dioxopiperidine, 3-(4-amino-1-oxo-1,3-dihydro-2H-isoindol-2-yl)-2,6-piperidinedione, or 4-amino-2-(6-hydroxy-2-oxo-2,3,4,5-tetrahydropyridin-3-yl)-2,3-dihydro-1H-isoindol-1-one.

Without being bound by any particular theory, lenalidomide as described herein may function as described in the embodiments below. In certain embodiment, lenalidomide as described herein and the protein as described in Section 5.1 or Section 5.2 may activate NK cells to a greater degree than either of the protein and said lenalidomide alone. In certain embodiments, lenalidomide as described herein and the protein as described herein may activate an immune cell other than NK cells to a greater degree than either of the protein and said lenalidomide alone. In certain embodiments, lenalidomide as described herein may have synergistic anti-cancer effect with the protein as described herein.

### 5.6 Pomalidomide

In one aspect, pomalidomide can be used with the combination treatments described herein. In certain embodiments, pomalidomide as described herein can be administered in combination with the protein that comprises an antigen-binding site that binds to an antigen on a natural killer (NK) cell as described in Section 5.1 in the methods of treating, managing, and/or preventing cancer described in Section 5.8. In certain embodiments, pomalidomide as described herein can be administered in combination with the protein that comprises an antigen-binding site that binds NKG2D as described in Section 5.2 in the in the methods of treating, managing, and/or preventing cancer described in Section 5.8.

In certain embodiments, pomalidomide (CAS Registry Number 19171-19-8) has the following structure:

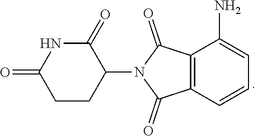 [SMILES: Nc1cccc2c1C(=O)N(C1CCC(=O)NC1=O)C2=O]

In certain embodiments, pomalidomide as described herein is referred to as 4-amino-2-(2,6-dioxopiperidin-3-yl)isoindoline-1,3-dione, 4-Amino-2-(2,6-dioxo-3-piperidyl)isoindoline-1,3-dione, 3-Amino-N-(2,6-dioxo-3-piperidyl)phthalimide, 4-amino-2-(2,6-dioxo-3-piperidinyl)-1H-Isoindole-1,3(2H)-dione, 4-amino-2-(2,6-dioxopiperidin-3-yl)-1H-isoindole-1,3(2H)-dione, 4-amino-2-(2,6-dioxopiperidin-3-yl)isoindole-1,3-dione, 3-amino-N-(2,6-dioxo-3-piperidyl)phthalamide, 3-Aminophthalimidoglutarimide, 1,3-dioxo-2-(2,6-dioxopiperidin-3-yl)-4-aminoisoindoline, 4-amino-2-(2,6-dioxo(3-piperidyl))isoindoline-1,3-dione, 4-Amino-2-(2,6-dioxo-3-piperidinyl)isoindole-1,3-dione, 4-Amino-2-(2,6-dioxo-3-piperidyl) isoindoline-1,3-dione, or 4-amino-2-(2,6-dioxo-piperidin-3-yl)isoindole-1,3-dione.

Without being bound by any particular theory, pomalidomide as described herein may function as described in the embodiments below. In certain embodiment, pomalidomide as described herein and the protein as described in Section 5.1 or Section 5.2 may activate NK cells to a greater degree than either of the protein and said pomalidomide alone. In certain embodiments, pomalidomide as described herein and the protein as described herein may activate an immune cell other than NK cells to a greater degree than either of the protein and said pomalidomide alone. In certain embodiments, pomalidomide as described herein may have synergistic anti-cancer effect with the protein as described herein.

### 5.7 CC-92480

In one aspect, CC-92480 can be used with the combination treatments described herein. In certain embodiments, CC-92480 as described herein can be administered in combination with the protein that comprises an antigen-binding site that binds to an antigen on a natural killer (NK) cell as described in Section 5.1 in the methods of treating, managing, and/or preventing cancer described in Section 5.8. In certain embodiments, CC-92480 as described herein can be administered in combination with the protein that comprises an antigen-binding site that binds NKG2D as described in Section 5.2 in the in the methods of treating, managing, and/or preventing cancer described in Section 5.8.

In certain embodiments, CC-92480 (CAS Registry Number 2259648-80-9) has the following structure:

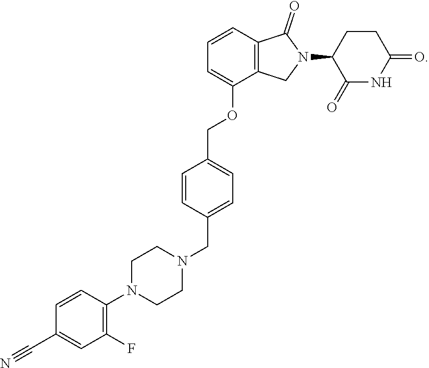 [SMILES: N#Cc1ccc(N2CCN(Cc3ccc(COc4cccc5c4CN([C@H]4CCC(=O)NC4=O)C5=O)cc3)CC2)c(F)c1]

In certain embodiments, CC-92480 as described herein is referred to as (S)-4-(4-(4-(((2-(2,6-dioxopiperidin-3-yl)-1-oxoisoindolin-4-yl)oxy)methyl)benzyl)piperazin-1-yl)-3-fluorobenzonitrile, 4-[4-[[4-[[2-[(3S)-2,6-dioxopiperidin-3-yl]-1-oxo-3H-isoindol-4-yl]oxymethyl]phenyl]methyl]piperazin-1-yl]-3-fluorobenzonitrile, or (83S)-12-fluoro-71,82,86-trioxo-71,73-dihydro-6-oxa-7(4,2)-isoindola-2(1,4)-piperazina-8(3)-piperidina-1(1),4(1,4)-dibenzenaoctaphane-14-carbonitrile.

Without being bound by any particular theory, CC-92480 as described herein may function as described in the embodiments below. In certain embodiment, CC-92480 as described herein and the protein as described in Section 5.1 or Section 5.2 may activate NK cells to a greater degree than either of the protein and said CC-92480 alone. In certain embodiments, CC-92480 as described herein and the protein as described herein may activate an immune cell other than NK cells to a greater degree than either of the protein and said CC-92480 alone. In certain embodiments, CC-92480 as described herein may have synergistic anti-cancer effect with the protein as described herein.

### 5.8 Method of Treating, Managing and/or Preventing Diseases

Provided herein are methods of treating, managing and/or preventing cancer comprising administering to a subject a therapeutically or prophylactically effective combination of (a) a protein that comprises (i) an antigen-binding site that binds an antigen on a natural killer (NK) cell, and (ii) an antigen-binding site that binds an antigen on a cell of said cancer as described in Section 5.1, and (b) iberdomide (CC-220), avadomide (CC-122), lenalidomide, pomalidomide, or CC-92480 as described in Sections 5.3 to 5.7.

Also provided herein are methods of treating, managing and/or preventing cancer comprising administering to a subject a therapeutically or prophylactically effective combination of (a) a protein that comprises (i) an antigen-binding site that binds NKG2D, and (ii) an antigen-binding site that binds an antigen on a cell of said cancer, and (ii) an antigen-binding site that binds an antigen on a cell of said cancer as described in Section 5.2; and (b) iberdomide (CC-220), avadomide (CC-122), lenalidomide, pomalidomide, or CC-92480 as described in Sections 5.3 to 5.7.

In certain embodiments, the protein described herein and lenalidomide, pomalidomide, avadomide, iberdomide, or CC-92480 can have synergistic anti-cancer effect. In certain embodiments, the protein described herein and lenalidomide, pomalidomide, avadomide, iberdomide, or CC-92480 can have additive anti-cancer effect. In certain embodiments, the protein described herein and lenalidomide, pomalidomide, avadomide, iberdomide, or CC-92480 not have an antagonistic anti-cancer effect. The synergistic, additive, or antagonistic effect may be determined by methods known in the art, for example methods based on multiple drug effect equations and quantifications by the combination index (CI).

In certain embodiments, the methods provided herein treat cancer in a subject. In certain embodiments, the methods provided herein manage cancer in a subject. In certain embodiments, the methods provided herein can result in progression-free survival (PFS). In certain embodiments, the methods provided herein can result in event-free survival (EFS). In certain embodiments, the methods provided herein can result in disease-free survival (DFS).

In certain embodiments, the methods provided herein can result in improvement in complete response rate (CRR). In certain embodiments, the methods provided herein can result in improvement in progression-free survival (PFS). In certain embodiments, the methods provided herein can result in an improvement in overall response rate. In certain embodiments, the methods provided herein can result in an improvement in objective response rate. In certain embodiments, the methods provided herein can result in an improvement in event-free survival (EFS). In certain embodiments, the methods provided herein can result in an improvement in disease-free survival (DFS). In certain embodiments, the methods provided herein can result in an improvement in overall survival (OS). In certain embodiments, the methods provided herein can result in reduced adverse events (AEs). In certain embodiments, the methods provided herein can result in an improvement in duration of response (DOR). In certain embodiments, the methods provided herein can result in an improvement in health-rated quality of life. In certain embodiments, the methods provided herein can result in an improvement in time to response (TTR). In certain embodiments, the methods provided herein can result in an improvement in time to progression (TTP). In certain embodiments, the methods provided herein can result in an improvement in time-to-treatment failure (TTF). In certain embodiments, the methods provided herein can result in an improvement in very good partial response or better rate (VGPR). In certain embodiments, one or more of the improvements described above are in comparison to the results from no treatment, the methods of treating or managing by administering one of the agents in the combination alone, or existing methods of treating cancer.

In certain embodiments in which the disease, disorder or condition is multiple myeloma, administration of the combination of the protein described herein and lenalidomide, pomalidomide, avadomide, iberdomide, or CC-92480 to a subject with multiple myeloma can result in an improvement of response by the subject from no response (NR) to at least partial response (PR); from PR to at least very good partial response (VGPR); from VGPR to at least complete response (CR); or from CR to strict complete response (sCR), as compared to administration of said protein or said lenalidomide, pomalidomide, avadomide, iberdomide, or CC-92480, alone. In specific embodiments, said subject has relapsed after therapy with said protein alone, or with said lenalidomide, pomalidomide, avadomide, iberdomide, or CC-92480 alone. In specific embodiments, the subject had achieved no response to administration of said protein, or said lenalidomide, pomalidomide, avadomide, iberdomide, or CC-92480, and improved to at least PR after administration of a combination of said protein and said lenalidomide, pomalidomide, avadomide, iberdomide, or CC-92480 the subject had achieved PR after administration of said protein, or said lenalidomide, pomalidomide, avadomide, iberdomide, or CC-92480, and improved to at least VGPR after administration of a combination of said protein and said lenalidomide, pomalidomide, avadomide, iberdomide, or CC-92480; the subject had achieved VGPR after administration of said protein, or said lenalidomide, pomalidomide, avadomide, iberdomide, or CC-92480, and improved to at least CR after administration of a combination of said protein and said lenalidomide, pomalidomide, avadomide, iberdomide, or CC-92480; or the subject had achieved CR after administration of said protein, or said lenalidomide, pomalidomide, avadomide, iberdomide, or CC-92480, and improved to at least sCR after administration of a combination of said protein and said lenalidomide, pomalidomide, avadomide, iberdomide, or CC-92480.

In certain embodiments, if available, standardized criteria can be used to ascertain response. A variety of response criteria have been considered appropriate (e.g., RECIST criteria) (Therasse et al, J Natl Cancer Inst, 92: 205-16 (2000)). In certain embodiments, for certain indications (e.g., non-Hodgkin's lymphoma), Lugano classification may be used (e.g., Cheson et al, J Clin Oncol, 32(27):3059-3068 (2014)). In certain embodiments, for certain indications (e.g., multiple myeloma), efficacy endpoint may be based on International Myeloma Working Group (IMWG) criteria. In certain embodiments, for certain indications (e.g., mantle cell lymphoma), response can be determined based on review of radiographic scans according to a modified version of the International Workshop Lymphoma Response Criteria (Cheson et al, J Clin Oncol, 17(4):1244 (1999)). In certain embodiments, for certain indications (e.g., follicular or marginal zone lymphoma), efficacy can be established in the intent-to-treat (ITT) population based on progression-free survival using International Working Group response criteria (e.g. as modified in 2007).

In certain embodiments, the methods provided herein may be used to prevent cancer in a subject. In certain embodiments, the methods may be used to prevent cancer in a healthy subject who has never been diagnosed with cancer. In certain embodiments, the methods may be used to prevent cancer reoccurring in a subject who has survived cancer.

### 5.8.1 Diseases

The methods may be used to treat a variety of cancers. In some embodiments, the cells of the cancer express BCMA. The methods of treating, managing and/or preventing can be characterized according to the cancer to be treated, managed or prevented.

In certain other embodiments, the cancer is leukemia, myeloma, non-Hodgkin's lymphoma (NHL), Hodgkin's lymphoma (HL), and Waldenstrom's macroglobulinemia.

In certain other embodiments, the cancer is non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma (DLBCL), primary mediastinal B-cell lymphoma, follicular lymphoma, small lymphocytic lymphoma, mantle cell lymphoma (MCL), marginal zone B-cell lymphoma, extranodal marginal zone B-cell lymphoma, marginal zone lymphoma (MZL), primary cutaneous anaplastic large cell lymphoma, follicular large cell lymphoma, nodal marginal zone B-cell lymphoma, splenic marginal zone B-cell lymphoma, Burkitt lymphoma, lymphoplasmacytic lymphoma, intravascular large B-cell lymphoma, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma. In certain embodiments, the cancer is classic Hodgkin lymphoma (cHL), or nodular lymphocyte-predominant Hodgkin lymphoma. In certain embodiments, the non-Hodgkin's lymphoma is lymphomatoid granulomatosis, hairy cell leukemia, post-transplantation lymphoproliferative disorder, true histiocytic lymphoma, primary effusion lymphoma, or plasmablastic lymphoma. In certain embodiments, the non-Hodgkin's lymphoma is follicle center lymphoma.

In certain embodiments, the cancer is multiple myeloma (MM), acute lymphoblastic leukemia (ALL), chronic lymphocytic leukemia, B cell lymphomas, chronic myeloid leukemia (CIVIL), chronic myelomonocytic leukemia (CMML), or acute myeloid leukemia (AML). In certain embodiments, the cancer is chronic lymphocytic leukemia/small lymphocytic lymphoma (CLL/SLL). In certain embodiments, the cancer is acute myeloid leukemia (AML).

In certain embodiments, the cancer is multiple myeloma. In certain embodiments, the cancer is mantle cell lymphoma (MCL). In certain embodiments, the cancer is follicular lymphoma (FL). In certain embodiments, the cancer is marginal zone lymphoma (MZL). In certain other embodiments, the cancer is non-Hodgkin's lymphoma. In certain embodiments, the cancer is diffuse B-cell lymphoma. In certain embodiments, the cancer is diffuse large B-cell lymphoma (DLBCL). In certain embodiments, the DLBCL is germinal center B-cell (GCB) DLBCL. In certain embodiments, the DLBCL is activated B-cell (ABC) DLBCL.

The cancer can be characterized according to the presence of a particular antigen expressed on the surface of the cancer cell. In certain embodiments, the cancer cell expresses one or more of the following in addition to BCMA: CD2, CD19, CD20, CD30, CD38, CD40, CD52, CD70, EGFR/ERBB1, IGF1R, HER3/ERBB3, HER4/ERBB4, MUC1, cMET, SLAMF7, PSCA, MICA, MICB, TRAILR1, TRAILR2, MAGE-A3, B7.1, B7.2, CTLA4, and/or PD1.

In certain embodiments, the cancer is newly diagnosed. In certain embodiments, the methods for provided herein may be used in a treatment-naive subject, i.e., a subject that has not yet received treatment.

In certain embodiments, the cancer is relapsed or refractory. In certain embodiments, the subject has failed at least one prior therapy. In certain embodiments, the methods provided herein may be used a subject that has not responded to standard treatment.

In certain embodiments, the subject is less than 18 years old. In certain embodiments, the subject is 18 years old or older. In certain embodiments, the methods may be used to treat a subject regardless of the subject's age, although some diseases or disorders are more common in certain age groups.

In certain embodiments, the methods may be used to treat a subject who has undergone surgery in an attempt to treat the disease or condition at issue, as well as one who has not. Because subjects with cancer have heterogeneous clinical manifestations and varying clinical outcomes, the treatment given to as subject may vary, depending on one's prognosis. The skilled clinician will be able to readily determine without undue experimentation specific secondary agents, types of surgery, and types of non-drug based standard therapy that can be effectively used to treat an individual patient with cancer.

### 5.8.2 Methods of Treating, Managing or Preventing

(i) Combinations

In certain embodiments, the methods of treating, managing and/or preventing cancer provided herein comprise administering to a subject (a) a protein that comprises (i) an antigen-binding site that binds an antigen on a natural killer (NK) cell, and (ii) an antigen-binding site that binds an antigen on a cell of said cancer, and (b) iberdomide (CC-220).

In certain embodiments, the methods provided herein comprise administering to a subject (a) a protein that comprises (i) an antigen-binding site that binds an antigen on a natural killer (NK) cell, and (ii) an antigen-binding site that binds an antigen on a cell of said cancer, and (b) avadomide (CC-122).

In certain embodiments, the methods of treating, managing and/or preventing cancer provided herein comprise administering to a subject (a) a protein that comprises (i) an antigen-binding site that binds an antigen on a natural killer (NK) cell, and (ii) an antigen-binding site that binds an antigen on a cell of said cancer, and (b) lenalidomide.

In certain embodiments, the methods of treating, managing and/or preventing cancer provided herein comprise administering to a subject (a) a protein that comprises (i) an antigen-binding site that binds an antigen on a natural killer (NK) cell, and (ii) an antigen-binding site that binds an antigen on a cell of said cancer, and (b) pomalidomide. In certain embodiments, the methods of treating, managing and/or preventing cancer provided herein do not comprise administering IL-2.

In certain embodiments, the methods provided herein comprise administering to a subject (a) a protein that comprises (i) an antigen-binding site that binds an antigen on a natural killer (NK) cell, and (ii) an antigen-binding site that binds an antigen on a cell of said cancer, and (b) CC-92480.

In certain embodiments, the methods of treating, managing and/or preventing cancer provided herein comprise administering to a subject (a) a protein that comprises (i) an antigen-binding site that binds an antigen on a natural killer (NK) cell, and (ii) an antigen-binding site that binds an antigen on a cell of said cancer, and (b) two agents selected from iberdomide (CC-220), avadomide (CC-122), lenalidomide, pomalidomide, and CC-92480.

In certain embodiments, the methods of treating, managing and/or preventing cancer provided herein comprise administering to a subject (a) a protein that comprises (i) an antigen-binding site that binds an antigen on a natural killer (NK) cell, and (ii) an antigen-binding site that binds an antigen on a cell of said cancer, and (b) three agents selected from iberdomide (CC-220), avadomide (CC-122), lenalidomide, pomalidomide, and CC-92480.

In certain embodiments, the methods of treating, managing and/or preventing cancer provided herein comprise administering to a subject (a) a protein that comprises (i) an antigen-binding site that binds NKG2D, and (ii) an antigen-binding site that binds an antigen on a cell of said cancer, and (ii) an antigen-binding site that binds an antigen on a cell of said cancer; and (b) iberdomide (CC-220).

In certain embodiments, the methods of treating, managing and/or preventing cancer provided herein comprise administering to a subject (a) a protein that comprises (i) an antigen-binding site that binds NKG2D, and (ii) an antigen-binding site that binds an antigen on a cell of said cancer, and (ii) an antigen-binding site that binds an antigen on a cell of said cancer; and (b) avadomide (CC-122).

In certain embodiments, the methods of treating, managing and/or preventing cancer provided herein comprise administering to a subject (a) a protein that comprises (i) an antigen-binding site that binds NKG2D, and (ii) an antigen-binding site that binds an antigen on a cell of said cancer, and (ii) an antigen-binding site that binds an antigen on a cell of said cancer; and (b) lenalidomide.

In certain embodiments, the methods of treating, managing and/or preventing cancer provided herein comprise administering to a subject (a) a protein that comprises (i) an antigen-binding site that binds NKG2D, and (ii) an antigen-binding site that binds an antigen on a cell of said cancer, and (ii) an antigen-binding site that binds an antigen on a cell of said cancer; and (b) pomalidomide. In certain embodiments, the methods of treating, managing and/or preventing cancer provided herein do not comprise administering IL-2.

In certain embodiments, the methods of treating, managing and/or preventing cancer provided herein comprise administering to a subject (a) a protein that comprises (i) an antigen-binding site that binds NKG2D, and (ii) an antigen-binding site that binds an antigen on a cell of said cancer, and (ii) an antigen-binding site that binds an antigen on a cell of said cancer; and (b) CC-92480.

In certain embodiments, the methods of treating, managing and/or preventing cancer provided herein comprise administering to a subject (a) a protein that comprises (i) an antigen-binding site that binds NKG2D, and (ii) an antigen-binding site that binds an antigen on a cell of said cancer, and (b) two agents selected from iberdomide (CC-220), avadomide (CC-122), lenalidomide, pomalidomide, and CC-92480.

In certain embodiments, the methods of treating, managing and/or preventing cancer provided herein comprise administering to a subject (a) a protein that comprises (i) an antigen-binding site that binds NKG2D, and (ii) an antigen-binding site that binds an antigen on a cell of said cancer, and (b) three agents selected from iberdomide (CC-220), avadomide (CC-122), lenalidomide, pomalidomide, and CC-92480.

In certain embodiments, the methods provide herein further comprise administering a therapy conventionally used to treat or manage cancer. Examples of such conventional therapies include, but are not limited to, surgery, chemotherapy, radiation therapy, hormonal therapy, biological therapy and immunotherapy.

In certain embodiments, the methods provide herein further comprise administering in combination or alternation with a therapeutically or prophylactically effective amount of one or more additional therapeutic agents, for example a third agent. In certain embodiments, the additional therapeutic agent is dexamethasone, iberdomide, avadomide, lenalidomide, pomalidomide, or CC-92480.

(ii) Doses

The amount of (a) the protein as described in Section 5.1 or Section 5.2 and (b) iberdomide (CC-220), avadomide (CC-122), lenalidomide, pomalidomide, or CC-92480 may be selected in order to achieve a desired combined therapeutic effect. In certain embodiments, the combination of (a) the protein as described in Section 5.1 or Section 5.2 and (b) iberdomide (CC-220), avadomide (CC-122), lenalidomide, pomalidomide or CC-92480 is administered in a therapeutically or prophylactically effective amount. In certain embodiments, while the combination is administered in a therapeutically or prophylactically effective amount, both (a) the protein as described in Section 5.1 or Section 5.2 and (b) iberdomide (CC-220), avadomide (CC-122), lenalidomide, pomalidomide, or CC-92480 are administered in a therapeutically or prophylactically effective amount. In certain embodiments, while the combination is in a therapeutically or prophylactically effective amount, one of (a) the protein as described in Section 5.1 or Section 5.2 and (b) iberdomide (CC-220), avadomide (CC-122), lenalidomide, pomalidomide, or CC-92480 is administered in a therapeutically or prophylactically effective amount. In certain embodiments, while the combination is in a therapeutically or prophylactically effective amount, neither (a) the protein as described in Section 5.1 or Section 5.2 nor (b) iberdomide (CC-220), avadomide (CC-122), lenalidomide, pomalidomide, or CC-92480 alone is administered in a therapeutically or prophylactically effective amount.

In certain embodiments, the protein as described herein is administered in a therapeutically or prophylactically effective amount. In certain embodiments, the protein provided herein is administered via intravenous, intraarterial, intraperitoneal, intramuscular, subcutaneous, intrapleural, intrathecal, or intracavitary route, or by perfusion through a catheter or by direct intralesional injection.

In certain embodiments, the protein is administered once or more times daily, weekly, monthly or yearly, or even once every 2 to 20 years. In certain embodiments, the protein is administered on certain days in a 28-day cycle.

In certain embodiments, the protein is administered in an amount from about 0.005 mg to about 5,000 mg per day, from about 0.01 mg to about 4,000 mg per day, from about 0.1 mg to about 3,000 mg per day, from about 0.5 mg to about 2,000 mg per day, from about 0.5 mg to about 1,000 mg per day, from about 0.5 mg to about 900 mg per day, from about 1 mg to about 800 mg per day, from about 1 mg to about 700 mg per day, from about 1 mg to about 600 mg per day, from about 2 mg to about 500 mg per day, from about 5 mg to about 400 mg per day, from about 10 mg to about 300 mg per day, from about 20 mg to about 200 mg per day, or from about 20 mg to about 100 mg per day.

In certain embodiments, the protein is administered in amounts based on body weight. Example amounts based on body weight are from about 0.01 μg to about 100 mg per kg of body weight, such as about 0.01 μg to about 100 mg/kg of body weight, about 0.01 μg to about 50 mg/kg of body weight, about 0.01 μg to about 10 mg/kg of body weight, about 0.01 μg to about 1 mg/kg of body weight, about 0.01 μg to about 100 μg/kg of body weight, about 0.01 μg to about 50 μg/kg of body weight, about 0.01 μg to about 10 μg/kg of body weight, about 0.01 μg to about 1 μg/kg of body weight, about 0.01 μg to about 0.1 μg/kg of body weight, about 0.1 μg to about 100 mg/kg of body weight, about 0.1 μg to about 50 mg/kg of body weight, about 0.1 μg to about 10 mg/kg of body weight, about 0.1 μg to about 1 mg/kg of body weight, about 0.1 μg to about 100 μg/kg of body weight, about 0.1 μg to about 10 μg/kg of body weight, about 0.1 μg to about 1 μg/kg of body weight, about 1 μg to about 100 mg/kg of body weight, about 1 μg to about 50 mg/kg of body weight, about 1 μg to about 10 mg/kg of body weight, about 1 μg to about 1 mg/kg of body weight, about 1 μg to about 100 μg/kg of body weight, about 1 μg to about 50 μg/kg of body weight, about 1 μg to about 10 μg/kg of body weight, about 10 μg to about 100 mg/kg of body weight, about 10 μg to about 50 mg/kg of body weight, about 10 μg to about 10 mg/kg of body weight, about 10 μg to about 1 mg/kg of body weight, about 10 μg to about 100 μg/kg of body weight, about 10 μg to about 50 μg/kg of body weight, about 50 μg to about 100 mg/kg of body weight, about 50 μg to about 50 mg/kg of body weight, about 50 μg to about 10 mg/kg of body weight, about 50 μg to about 1 mg/kg of body weight, about 50 μg to about 100 μg/kg of body weight, about 100 μg to about 100 mg/kg of body weight, about 100 μg to about 50 mg/kg of body weight, about 100 μg to about 10 mg/kg of body weight, about 100 μg to about 1 mg/kg of body weight, about 1 mg to about 100 mg/kg of body weight, about 1 mg to about 50 mg/kg of body weight, about 1 mg to about 10 mg/kg of body weight, about 10 mg to about 100 mg/kg of body weight, about 10 mg to about 50 mg/kg of body weight, and about 50 mg to about 100 mg/kg of body weight.

In certain embodiments, iberdomide (CC-220) is administered in a therapeutically or prophylactically effective amount. In preferred embodiments, iberdomide is administered in an amount of about 0.1 mg to about 2 mg per day. In preferred embodiments, iberdomide is administered on certain days of a 28-day cycle, for example days 1 to 21 of a 28-day cycle. In preferred embodiments, iberdomide is administered orally. In certain embodiments, iberdomide is administered according to the locally approved label or Pharmacy manual for preparation, administration, and storage information.

In certain embodiments, avadomide (CC-122) is administered in a therapeutically or prophylactically effective amount. In certain embodiments, avadomide is administered in an amount of about 2 mg to about 50 mg per day. In preferred embodiments, avadomide is administered in an amount of about 2 mg, about 3 mg, about 4 mg, and about 5 mg per day. In preferred embodiments, avadomide is administered on certain days of a 7-day cycle, for example days 1 to 5 of a 7-day cycle. In preferred embodiments, avadomide is administered on certain days of a 28-day cycle, for example days 1 to 21 of a 28-day cycle. In preferred embodiments, avadomide is administered orally. In certain embodiments, avadomide is administered according to the locally approved label or Pharmacy manual for preparation, administration, and storage information.

In certain embodiments, lenalidomide is administered in a therapeutically or prophylactically effective amount. In preferred embodiments, lenalidomide is administered in an amount of about 10 mg to about 25 mg per day. In preferred embodiments, lenalidomide is administered on certain days of a 28-day cycle, for example days 1 to 21 of a 28-day cycle. In preferred embodiments, lenalidomide is administered orally. In certain embodiments, lenalidomide is administered according to the locally approved label or Pharmacy manual for preparation, administration, and storage information, for example, according to the label approved by the U.S. Food and Drug Administration of REVLIMID®.

In certain embodiments, pomalidomide is administered in a therapeutically or prophylactically effective amount. In preferred embodiments, pomalidomide is administered in an amount of about 3 mg to about 5 mg per day. In preferred embodiments, pomalidomide is administered on certain days of a 28-day cycle, for example days 1 to 21 of a 28-day cycle. In preferred embodiments, pomalidomide is administered orally. In certain embodiments, pomalidomide is administered according to the locally approved label or Pharmacy manual for preparation, administration, and storage information, for example, according to the label approved by the U.S. Food and Drug Administration of POMALYST®.

In certain embodiments, CC-92480 is administered in a therapeutically or prophylactically effective amount. In certain embodiments, CC-92480 is administered in an amount of about 0.1 mg to about 2 mg per day. In certain embodiments, CC-92480 is administered in an amount of about 0.3 mg, about 0.6 mg, about 0.8 mg, or about 1 mg once daily. In certain embodiments, CC-92480 is administered over a 21-day cycle. In certain embodiments, CC-92480 is administered over a 28-day cycle. In certain embodiments, CC-92480 is administered over a 21-day cycle from Cycle 1 to Cycle 8 and over a 28-day cycle from Cycle 9 onwards. In certain embodiments, CC-92480 is administered for 7 days followed by 7 days of rest, for 14 days followed by 7 days of rest, or for 21 days followed by 7 days of rest. In preferred embodiments, CC-92480 is administered orally. In certain embodiments, CC-92480 is administered according to the locally approved label or Pharmacy manual for preparation, administration, and storage information.

In certain embodiments, the methods described herein further comprises administration of dexamethasone. In certain embodiments, the recommended daily dose range of dexamethasone, for the conditions described herein lie within the range of from about 0.5 mg to about 100 mg per day, preferably given as a single once-a-day dose, or in divided doses throughout a day. In some embodiments, the dosage ranges from about 1 mg to about 100 mg per day. In other embodiments, the dosage ranges from about 0.5 mg to about 20 mg per day. Specific doses include 0.5 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 60 mg, 70 mg, 80 mg, 90 mg, or 100 mg per day.

(iii) Administration

The relative timing of administration of (a) the protein as described in Section 5.1 or Section 5.2 and (b) iberdomide (CC-220), avadomide (CC-122), lenalidomide, pomalidomide, or CC-92480 as described in Sections 5.3 to 5.7,
optionally with any addition agent may be selected in order to achieve a desired combined therapeutic effect. For example, when administering a combination therapy to a subject in need of such administration, the therapeutic agents in the combination, or a pharmaceutical composition or compositions comprising the therapeutic agents, may be administered in any order such as, for example, sequentially, concurrently, together, simultaneously and the like. Further, for example, the protein may be administered during a time when (b) iberdomide (CC-220), avadomide (CC-122), lenalidomide, pomalidomide, or CC-92480 exerts its prophylactic or therapeutic effect, or vice versa. Persons of ordinary skill in the art can estimate repetition rates for dosing based on measured residence times and concentrations of the targetable construct or complex in bodily fluids or tissues.

In certain embodiments, the protein provided herein is administered once per day from one day to six months, from one week to three months, from one week to four weeks, from one week to three weeks, or from one week to two weeks. In certain embodiments, the protein is administered once per day for one week, two weeks, three weeks, or four weeks.

In certain embodiments, the protein provided herein is administered daily. As used herein, the term “daily” or “per day” is intended to mean that an agent is administered once each day or more than once each day.

In certain embodiments, the protein provided herein is administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), one or more additional agents. In certain embodiments, the additional agent is iberdomide (CC-220), avadomide (CC-122), lenalidomide, pomalidomide, or CC-92480. In certain embodiments, the protein provided herein in combination with the one or more additional agents can also be administered on an alternating dosing schedule, with or without a resting period (e.g., no therapeutic agent is administered on certain days of the schedule). In certain embodiments, the administration of the protein described herein in combination with the one or more additional agents includes, but is not limited to, sequential administration and concomitant administration.

### 5.9 Compositions and/or Formulations

In another aspect, also provided herein are compositions and/or formulations comprising the protein as described in Section 5.1 or Section 5.2. Also provided herein are compositions and/or formulations comprising iberdomide (CC-220), avadomide (CC-122), lenalidomide, pomalidomide, and CC-92480 as described in Sections 5.3 to 5.7.

The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer R, Science 249:1527-1533, 1990.

The intravenous drug delivery formulation of the present disclosure may be contained in a bag, a pen, or a syringe. In certain embodiments, the bag may be connected to a channel comprising a tube and/or a needle. In certain embodiments, the formulation may be a lyophilized formulation or a liquid formulation. In certain embodiments, the formulation may freeze-dried (lyophilized) and contained in about 12-60 vials. In certain embodiments, the formulation may be freeze-dried and 45 mg of the freeze-dried formulation may be contained in one vial. In certain embodiments, the about 40 mg-about 100 mg of freeze-dried formulation may be contained in one vial. In certain embodiments, freeze dried formulation from 12, 27, or 45 vials are combined to obtained a therapeutic dose of the protein in the intravenous drug formulation. In certain embodiments, the formulation may be a liquid formulation and stored as about 250 mg/vial to about 1000 mg/vial. In certain embodiments, the formulation may be a liquid formulation and stored as about 600 mg/vial. In certain embodiments, the formulation may be a liquid formulation and stored as about 250 mg/vial.

In certain embodiments, the protein provided herein could exist in a liquid aqueous pharmaceutical formulation including a therapeutically or prophylactically effective amount of the protein in a buffered solution forming a formulation.

These compositions may be sterilized by conventional sterilization techniques, or may be sterile filtered. The resulting aqueous solutions may be packaged for use as-is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents. The composition in solid form can also be packaged in a container for a flexible quantity.

In certain embodiments, the present disclosure provides a formulation with an extended shelf life including the protein of the present disclosure, in combination with mannitol, citric acid monohydrate, sodium citrate, disodium phosphate dihydrate, sodium dihydrogen phosphate dihydrate, sodium chloride, polysorbate 80, water, and sodium hydroxide.

In certain embodiments, an aqueous formulation is prepared including the protein of the present disclosure in a pH-buffered solution. The buffer of this invention may have a pH ranging from about 4 to about 8, e.g., from about 4.5 to about 6.0, or from about 4.8 to about 5.5, or may have a pH of about 5.0 to about 5.2. Ranges intermediate to the above recited pH's are also intended to be part of this disclosure. For example, ranges of values using a combination of any of the above recited values as upper and/or lower limits are intended to be included. Examples of buffers that will control the pH within this range include acetate (e.g., sodium acetate), succinate (such as sodium succinate), gluconate, histidine, citrate and other organic acid buffers.

In certain embodiments, the formulation includes a buffer system which contains citrate and phosphate to maintain the pH in a range of about 4 to about 8. In certain embodiments the pH range may be from about 4.5 to about 6.0, or from about pH 4.8 to about 5.5, or in a pH range of about 5.0 to about 5.2. In certain embodiments, the buffer system includes citric acid monohydrate, sodium citrate, disodium phosphate dihydrate, and/or sodium dihydrogen phosphate dihydrate. In certain embodiments, the buffer system includes about 1.3 mg/mL of citric acid (e.g., 1.305 mg/mL), about 0.3 mg/mL of sodium citrate (e.g., 0.305 mg/mL), about 1.5 mg/mL of disodium phosphate dihydrate (e.g., 1.53 mg/mL), about 0.9 mg/mL of sodium dihydrogen phosphate dihydrate (e.g., 0.86), and about 6.2 mg/mL of sodium chloride (e.g., 6.165 mg/mL). In certain embodiments, the buffer system includes 1-1.5 mg/mL of citric acid, 0.25 to 0.5 mg/mL of sodium citrate, 1.25 to 1.75 mg/mL of disodium phosphate dihydrate, 0.7 to 1.1 mg/mL of sodium dihydrogen phosphate dihydrate, and 6.0 to 6.4 mg/mL of sodium chloride. In certain embodiments, the pH of the formulation is adjusted with sodium hydroxide.

A polyol, which acts as a tonicifier and may stabilize the protein, may also be included in the formulation. The polyol is added to the formulation in an amount which may vary with respect to the desired isotonicity of the formulation. In certain embodiments, the aqueous formulation may be isotonic. The amount of polyol added may also be altered with respect to the molecular weight of the polyol. For example, a lower amount of a monosaccharide (e.g., mannitol) may be added, compared to a disaccharide (such as trehalose). In certain embodiments, the polyol which may be used in the formulation as a tonicity agent is mannitol. In certain embodiments, the mannitol concentration may be about 5 to about 20 mg/mL. In certain embodiments, the concentration of mannitol may be about 7.5 to 15 mg/mL. In certain embodiments, the concentration of mannitol may be about 10-14 mg/mL. In certain embodiments, the concentration of mannitol may be about 12 mg/mL. In certain embodiments, the polyol sorbitol may be included in the formulation.

A detergent or surfactant may also be added to the formulation. Exemplary detergents include nonionic detergents such as polysorbates (e.g., polysorbates 20, 80 etc.) or poloxamers (e.g., poloxamer 188). The amount of detergent added is such that it reduces aggregation of the formulated antibody and/or minimizes the formation of particulates in the formulation and/or reduces adsorption. In certain embodiments, the formulation may include a surfactant which is a polysorbate. In certain embodiments, the formulation may contain the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th ed., 1996). In certain embodiments, the formulation may contain between about 0.1 mg/mL and about 10 mg/mL of polysorbate 80, or between about 0.5 mg/mL and about 5 mg/mL. In certain embodiments, about 0.1% polysorbate 80 may be added in the formulation.

In certain embodiments, the protein provided herein is formulated as a liquid formulation. The liquid formulation may be presented at a 10 mg/mL concentration in either a USP/Ph Eur type I 50R vial closed with a rubber stopper and sealed with an aluminum crimp seal closure. The stopper may be made of elastomer complying with USP and Ph Eur. In certain embodiments vials may be filled with 61.2 mL of the protein product solution in order to allow an extractable volume of 60 mL. In certain embodiments, the liquid formulation may be diluted with 0.9% saline solution.

In certain embodiments, the liquid formulation of the disclosure may be prepared as a 10 mg/mL concentration solution in combination with a sugar at stabilizing levels. In certain embodiments the liquid formulation may be prepared in an aqueous carrier. In certain embodiments, a stabilizer may be added in an amount no greater than that which may result in a viscosity undesirable or unsuitable for intravenous administration. In certain embodiments, the sugar may be disaccharides, e.g., sucrose. In certain embodiments, the liquid formulation may also include one or more of a buffering agent, a surfactant, and a preservative.

In certain embodiments, the pH of the liquid formulation may be set by addition of a pharmaceutically acceptable acid and/or base. In certain embodiments, the pharmaceutically acceptable acid may be hydrochloric acid. In certain embodiments, the base may be sodium hydroxide.

In certain embodiments, the protein provided herein can be deamidated. In certain embodiments, deamidation may occur during fermentation, harvest/cell clarification, purification, drug substance/drug product storage and during sample analysis.

In certain embodiments, the liquid formulation of the present disclosure may be preserved under conditions of pH and humidity to prevent deamination of the protein product.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

A preservative may be optionally added to the formulations herein to reduce bacterial action. The addition of a preservative may, for example, facilitate the production of a multi-use (multiple-dose) formulation.

Intravenous (IV) formulations may be the preferred administration route in particular instances, such as when a patient is in the hospital after transplantation receiving all drugs via the IV route. In certain embodiments, the liquid formulation is diluted with 0.9% Sodium Chloride solution before administration. In certain embodiments, the diluted drug product for injection is isotonic and suitable for administration by intravenous infusion.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

A preservative may be optionally added to the formulations herein to reduce bacterial action. The addition of a preservative may, for example, facilitate the production of a multi-use (multiple-dose) formulation.

The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

In certain embodiments, the protein provided herein could exist in a lyophilized formulation including the proteins and a lyoprotectant. The lyoprotectant may be sugar, e.g., disaccharides. In certain embodiments, the lyoprotectant may be sucrose or maltose. The lyophilized formulation may also include one or more of a buffering agent, a surfactant, a bulking agent, and/or a preservative.

The amount of sucrose or maltose useful for stabilization of the lyophilized drug product may be in a weight ratio of at least 1:2 protein to sucrose or maltose. In certain embodiments, the protein to sucrose or maltose weight ratio may be of from 1:2 to 1:5.

In certain embodiments, the pH of the formulation, prior to lyophilization, may be set by addition of a pharmaceutically acceptable acid and/or base. In certain embodiments the pharmaceutically acceptable acid may be hydrochloric acid. In certain embodiments, the pharmaceutically acceptable base may be sodium hydroxide.

Before lyophilization, the pH of the solution containing the protein of the present disclosure may be adjusted between 6 to 8. In certain embodiments, the pH range for the lyophilized drug product may be from 7 to 8.

In certain embodiments, a salt or buffer components may be added in an amount of 10 mM-200 mM. The salts and/or buffers are pharmaceutically acceptable and are derived from various known acids (inorganic and organic) with “base forming” metals or amines. In certain embodiments, the buffer may be phosphate buffer. In certain embodiments, the buffer may be glycinate, carbonate, citrate buffers, in which case, sodium, potassium or ammonium ions can serve as counterion.

In certain embodiments, a “bulking agent” may be added. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents.

A preservative may be optionally added to the formulations herein to reduce bacterial action. The addition of a preservative may, for example, facilitate the production of a multi-use (multiple-dose) formulation.

In certain embodiments, the lyophilized drug product may be constituted with an aqueous carrier. The aqueous carrier of interest herein is one which is pharmaceutically acceptable (e.g., safe and non-toxic for administration to a human) and is useful for the preparation of a liquid formulation, after lyophilization. Illustrative diluents include sterile water for injection (SWFI), bacteriostatic water for injection (BWFI), a pH buffered solution (e.g., phosphate-buffered saline), sterile saline solution, Ringer's solution or dextrose solution.

In certain embodiments, the lyophilized drug product of the current disclosure is reconstituted with either Sterile Water for Injection, USP (SWFI) or 0.9% Sodium Chloride Injection, USP. During reconstitution, the lyophilized powder dissolves into a solution.

In certain embodiments, the lyophilized protein product of the instant disclosure is constituted to about 4.5 mL water for injection and diluted with 0.9% saline solution (sodium chloride solution).

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The specific dose can be a uniform dose for each patient, for example, 50-5000 mg of protein. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al, Clinica Chimica Acta 308: 43-53 (2001); Steimer et al, Clinica Chimica Acta 308: 33-41 (2001)).

In certain embodiments, the compositions and/or formulations comprising the protein described herein further comprise an additional agent. In other embodiments, the protein described herein can be in a separated composition and/or formulation from the composition and/or formulation of the additional agent. In certain embodiments, the additional agent is iberdomide (CC-220), avadomide (CC-122), lenalidomide, pomalidomide, or CC-92480.

In certain embodiments, the compositions and/or formulations of iberdomide is described in the locally approved label or Pharmacy manual for preparation, administration, and storage information.

In certain embodiments, the compositions and/or formulations of avadomide is described in the locally approved label or Pharmacy manual for preparation, administration, and storage information.

In certain embodiments, the compositions and/or formulations of lenalidomide is described in the locally approved label or Pharmacy manual for preparation, administration, and storage information, for example, according to the label approved by the U.S Food and Drug Administration of REVLIMID®.

In certain embodiments, the compositions and/or formulations of pomalidomide is described in the locally approved label or Pharmacy manual for preparation, administration, and storage information, for example, according to the label approved by the U.S Food and Drug Administration of POMALYST®.

### 5.10 Assays

A person having ordinary skill in the art may use the assays as described herein and/or techniques known in the art to study the compositions and methods described herein, for example to evaluate the protein as described in Section 5.1 or Section 5.2, and/or iberdomide (CC-220), avadomide (CC-122), lenalidomide, pomalidomide, and CC-92480 as described in Sections 5.3 to 5.7. The examples provided in Section 6 also demonstrate in more detail how such assays may be used.

### 5.10.1 Flow Cytometry Analysis

Flow cytometry analysis can be used to identify cell populations. Specifically, flow cytometry analysis may be used to evaluate NK and T cell activation. For example, percentage of NK cells can be analyzed via flow cytometry gated on live CD3-CD19-CD14-CD56+CD16+NK cells.

### 5.10.2 In Vitro and/or Ex Vivo Assays on Immune Cell-Mediated Lysis

In vitro and/or ex vivo assays may be used to evaluate the ability to mediate and/or enhance immune cell-mediated lysis of target cells, for example NK cell-mediated lysis of cancer cells, of the protein as described herein alone, iberdomide (CC-220), avadomide (CC-122), lenalidomide, pomalidomide, or CC-92480 alone, or the combination of the protein and iberdomide (CC-220), avadomide (CC-122), lenalidomide, pomalidomide, or CC-92480. EC₅₀ values can be calculated based on titration of treatment concentrations. Maximum lysis can be evaluated. Synergistic effect can be evaluated in in vitro cell-based assays as well.

For example, ex vivo cytotoxicity and antibody-dependent cellular cytotoxicity (ADCC) assays may be used to evaluate the efficacy of the protein as described herein alone, iberdomide (CC-220), avadomide (CC-122), lenalidomide, pomalidomide, or CC-92480 alone, or the combination of the protein and iberdomide (CC-220), avadomide (CC-122), lenalidomide, pomalidomide, or CC-92480.

### 5.10.3 Animal Studies

In vivo animal studies using cancer animal models may be used to evaluate the anti-cancer effect of the protein as described herein in combination with one or more additional agents as described herein. Exemplary animal models include, but are not limited to, disseminated tumor models and blood cancer models. Exemplary species include, but are not limited to, mice, rats, zebrafish, rabbits, dogs, cats, non-human primates and pigs.

### 5.10.4 Clinical Studies

Clinical studies may be used to evaluate the methods of treating, managing and preventing cancer in a subject using the protein as described herein in combination with one or more additional agents as described herein.

Primary and secondary outcome measures that have been known or used in the art for particular indications may be evaluated. For example, for the treatment of diffuse large B-cell lymphoma (DLBCL), primary outcome measures may include maximum tolerated dose/maximum administered dose (MTD/MAD), frequency of dose-limiting toxicities (DLTs) associated with the addition of one agent to another agent, complete response rate (CRR), percentage of participants experiencing positron emission tomography (PET)-negative complete response (CR), whereas secondary outcome measures may include overall response rate, percentage of participants who achieve a PR or CR according to the Lugano criteria, Overall response rate by predictive gene signature, progression-free survival (PFS), event-free survival (EFS), overall survival (OS), and adverse events (AEs). For the treatment of relapsed/refractory B-cell malignancies, primary outcome measures may include dose-limiting toxicity rates and complete response rate (CRR); whereas secondary outcome measures may include progression-free survival (PFS), overall survival (OS), overall response rate, duration of response (DOR), event-free survival (EFS), pharmacokinetic parameters (for example, Cₘₐₓ, Tₘₐₓ and AUC), health-rated quality of life, quality of life evaluations, and adverse events (AEs). For the treatment of multiple myeloma, primary outcome measures may include maximum tolerated dose (MTD) and overall response rate; whereas secondary outcome measures may include overall response rate, time to response (TTR), duration of response (DOR), pharmacokinetic parameters (for example, Cₘₐₓ, Tₘₐₓ, t1/2, CLss/F, Vss/F and AUC), progression-free survival (PFS), overall survival (OS), very good partial response or better rate (VGPR), and adverse events (AEs).

### 6. EXAMPLES

### 6.1 Example 1: Iberdomide (CC-220), Avadomide (CC-122), and Lenalidomide Mediated Degradation of Aiolos and Ikaros in Primary NK Cells

Peripheral blood mononuclear cells (PBMCs) from healthy donors (n=7) were treated with dose titrations of iberdomide, avadomide, or lenalidomide for 5 hours. Intracellular staining of Aiolos and Ikaros in primary human NK cells was performed by flow cytometry gating on live, CD3-CD4-CD8-CD56+CD16+NK cell populations.

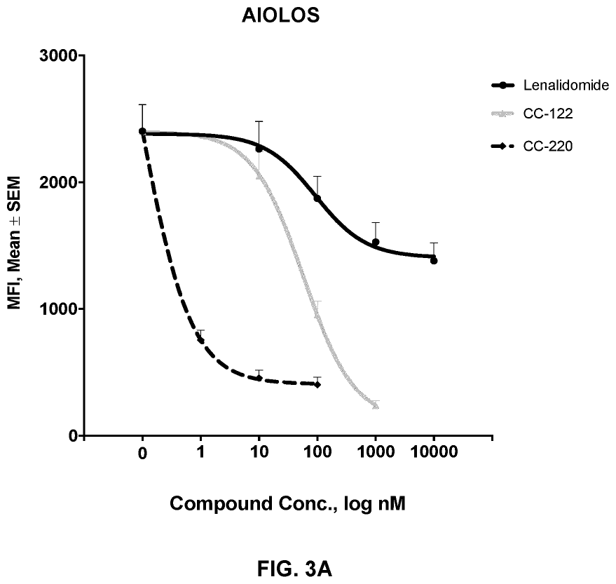

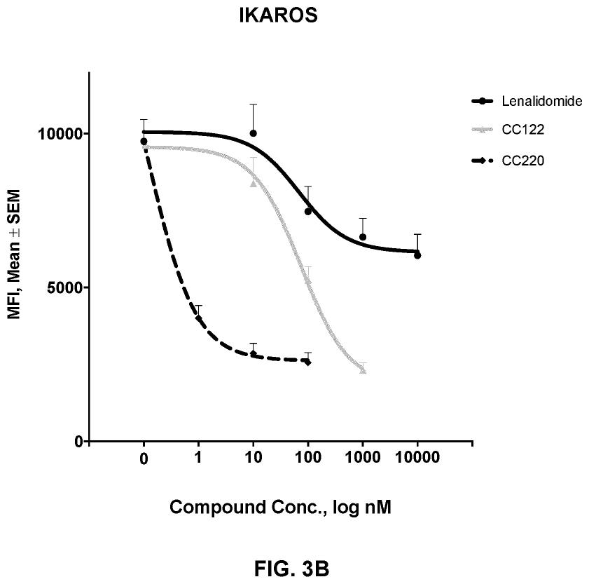

As shown in FIGS. 3A-3B, iberdomide, avadomide, or lenalidomide mediates degradation of Aiolos and Ikaros in primary NK cells.

### 6.2 Example 2: Avadomide (CC-122) and Lenalidomide Dose Dependently Enhanced NK Cell Proliferation in DLBCL Patient Samples

PBMCs from diffuse large B-cell lymphoma (DLBCL) patients (n=6) were treated with avadomide or lenalidomide for 1 hour prior to being stimulated with anti-CD3 for 3 days. Percentage of NK cells was analyzed via flow cytometry gated on live CD3−CD19−CD14−CD56+CD16+NK cells. Cytokinic NK cells were CD56^{high}CD16^{low} and cytotoxic NK cells were CD56^{low}CD16^{high} populations.

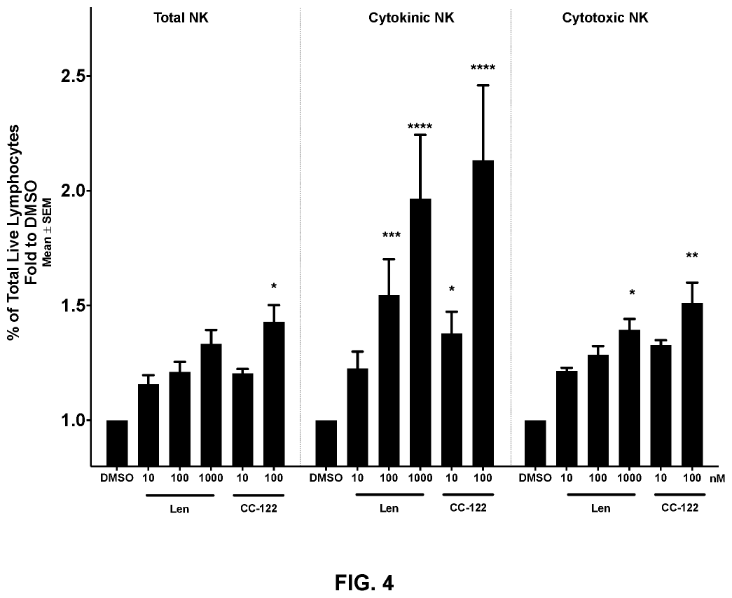

As shown in FIG. 4, avadomide and lenalidomide dose dependently enhanced NK cell proliferation in DLBCL patient samples.

### 6.3 Example 3: Avadomide (CC-122) and Lenalidomide Significantly Expanded NKG2D+ NK Cells in DLBCL Patient PBMC

PBMCs from diffuse large B-cell lymphoma (DLBCL) patients (n=6) were treated with avadomide or lenalidomide for 1 hour prior to being stimulated with anti-CD3 for 3 days. Surface expression of NKG2D was measured by flow cytometry gated on live CD3-CD19-CD14-CD56+CD16+NK cells.

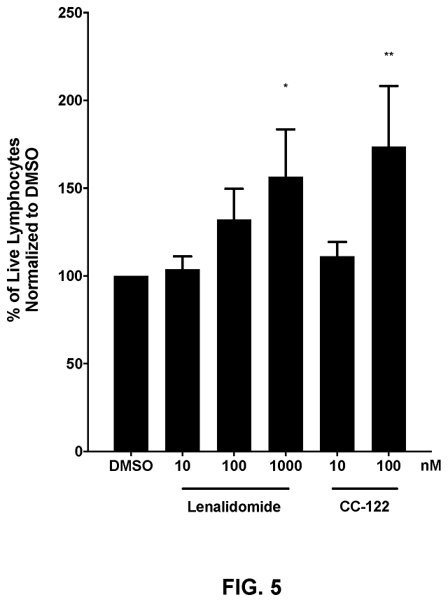

As shown in FIG. 5, avadomide and lenalidomide significantly expanded NKG2D+NK cells in DLBCL patient PBMC.

### 6.4 Example 4: Iberdomide (CC-220) Enhanced NK Cell Cytotoxicity Against CLL Cell Lines

Freshly isolated human NK cells from normal donors (n=4) were treated overnight with supernatants obtained from iberdomide treated and anti-CD3 stimulated PBMCs prior to co-culture with CSFE labeled chronic lymphocytic leukemia (CLL) cell lines for 4 hours. Live target cells were gated on CFSE+Annexin-Topro-population by flow cytometry.

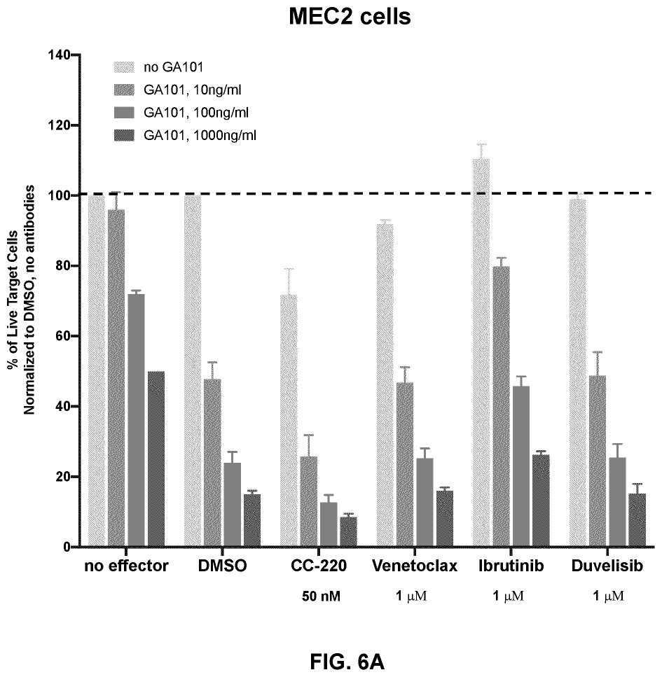

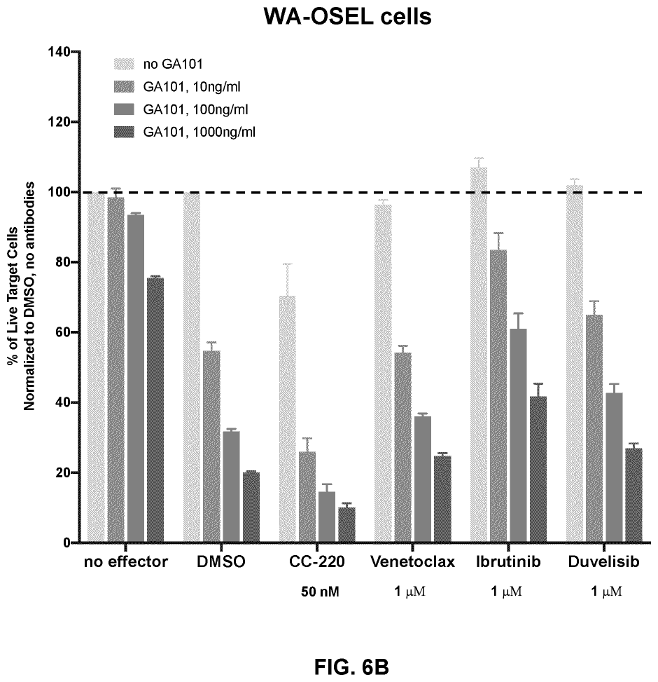

As shown in FIGS. 6A-6B, compared to other therapeutic agents such as venetoclax, ibrutinib, or duvelisib, iberdomide enhanced NK cell mediated cytotoxicity and antibody-dependent cellular cytotoxicity (ADCC) against CLL cell lines. In this example, obinutuzumab (GA101) was used to mediate ADCC.

### 6.5 Example 5: Avadomide (CC-122) Upregulated Antigen Presentation Molecules on Tumor Cells

Karpas 422, WSU-DLCL2, U2932 and TMD8 cells were treated with avadomide at a concentration of 0.1, 1, or 10 μM. The expression of Aiolos, Ikaros, IRG7 and IRF4 were measured by western blot.

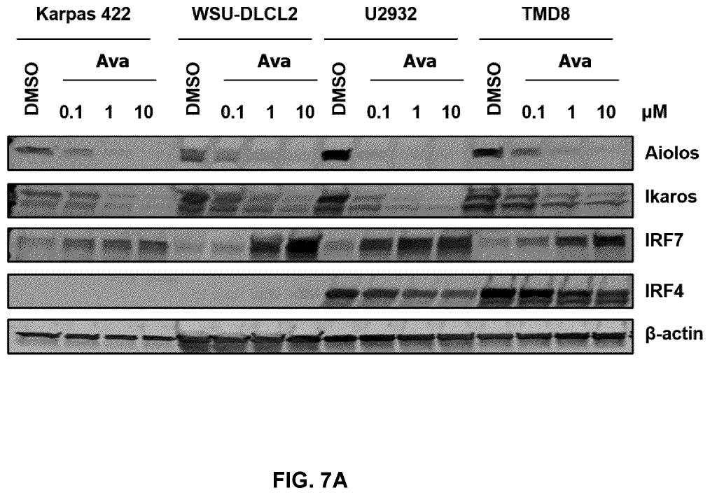

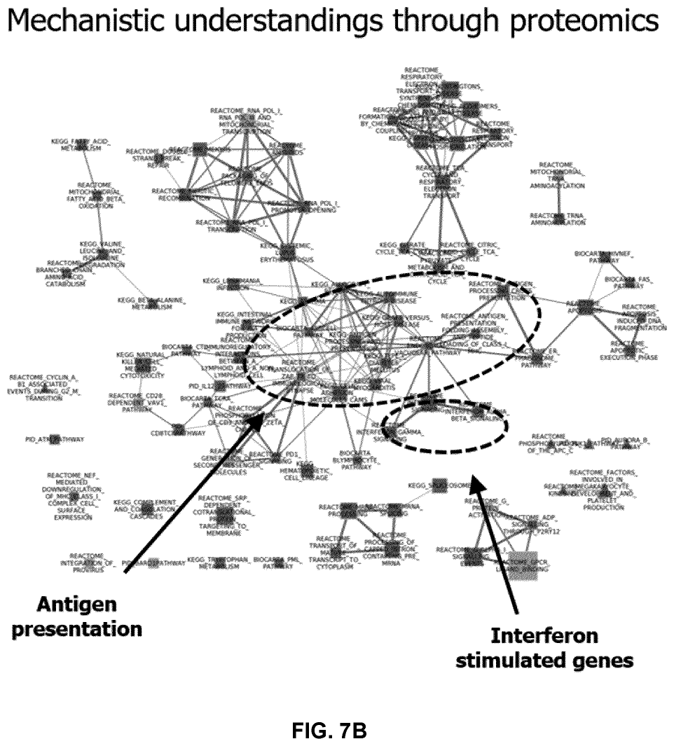

As shown in FIG. 7A, avadomide mediated degradation of Aiolos and Ikaros in these cells. The induction of interferon stimulated genes was correlated with the degradation of Aiolos and Ikaros. Degradation of Aiolos and Ikaros in DLBCL cells upregulated interferon stimulated genes, as well as molecules involved in antigen presentation. Such direct regulation was shown using Chromatin Immunoprecipitation sequencing (ChIP-seq) analysis and generic methods, see, e.g., Hagner et al, Blood, 126(6):779-789 (2015), which is incorporated by reference in its entirety.

FIG. 7B illustrates pathway analysis of proteins induced in CLL cells treated with avadomide.

### 6.6 Example 6: Aiolos and Ikaros Directly Regulated Expression of NKG2D Receptor MICA

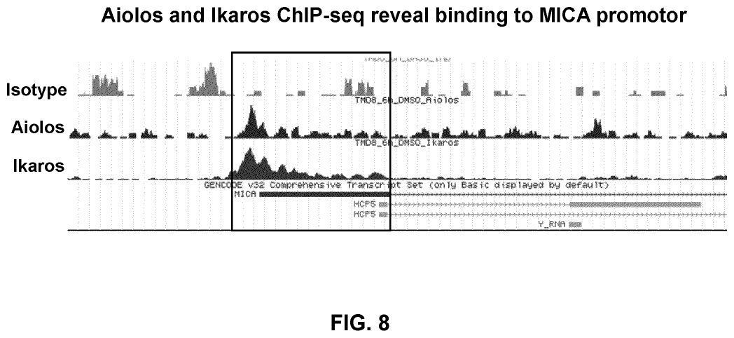

Chromatin Immunoprecipitation sequencing (ChIP-seq) analysis revealed that Aiolos and Ikaros were binding to MICA promotor (FIG. 8). Thus, Aiolos and Ikaros regulated the expression of MICA. Accordingly, degradation of Aiolos and Ikaros increased MICA expression, thereby enhancing NK cell cytotoxicity.

### 6.7 Example 7: Aiolos and Ikaros Directly Regulate Promoters of MHC I/II and Immune Recognition Genes in DLBCL Cell Lines

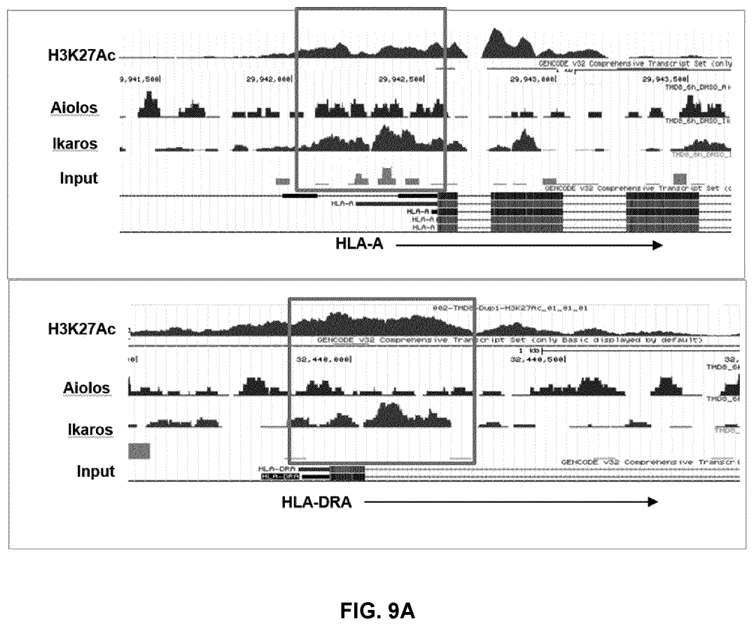

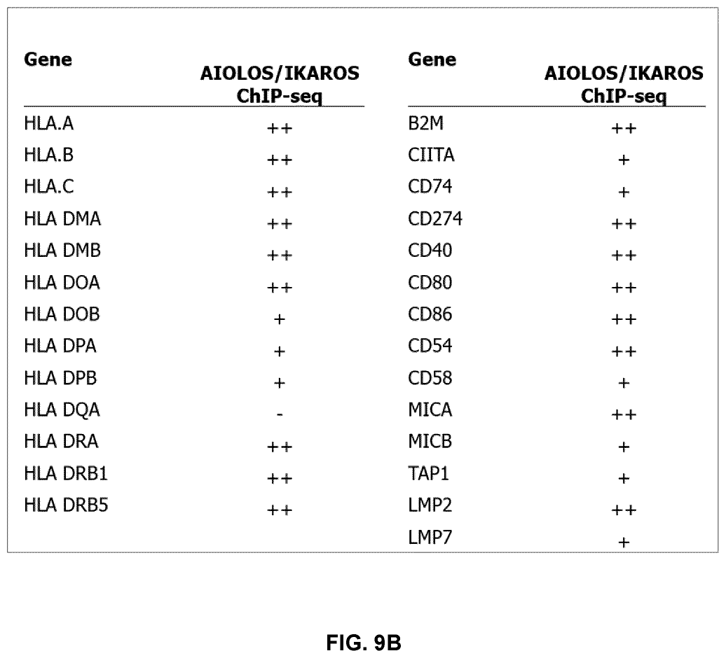

ChIP-seq analysis revealed that Aiolos and Ikaros were binding to the TSS of most of the MHC I/II and T/NK co-stimulatory genes, as well as those critically involved in antigen processing and presenting machinery in DLBCL cell lines (FIGS. 9A-9B).

### 6.8 Example 8: Avadomide (CC-122) Treatment Increased Expression of HLA-DR and CD86 in DLBCL Cell Lines

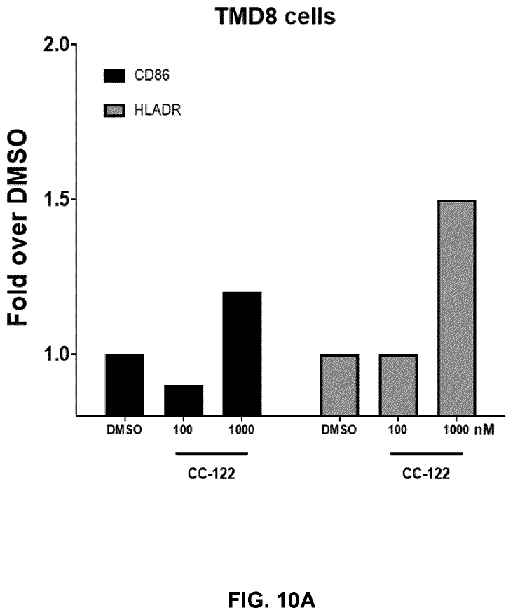

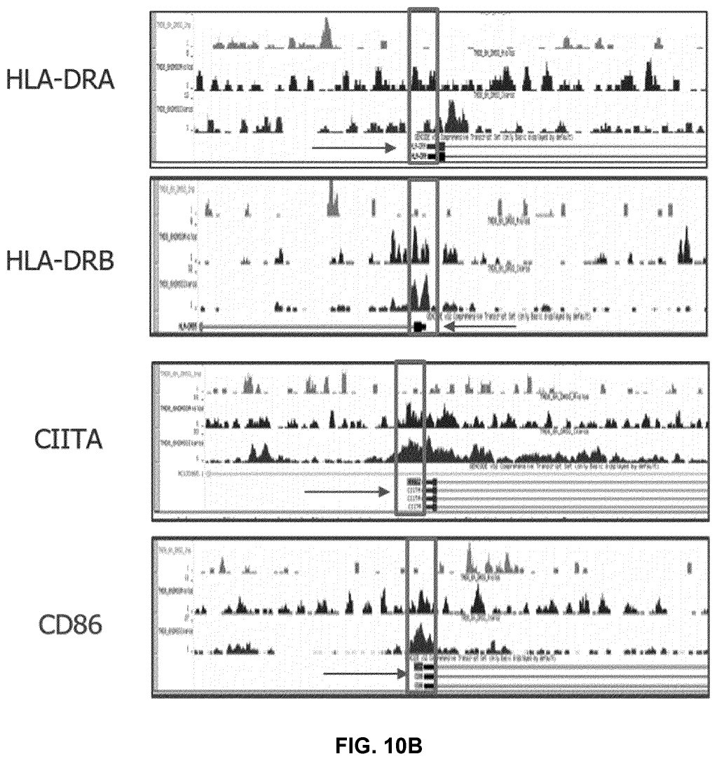

DLBCL cell line TMD8 was treated with avadomide for 48 hours. Flow cytometry analysis of the treated TMD8 cells revealed that avadomide-mediated Aiolos/Ikaros degradation enhanced expression of HLA-DR and CD86 (FIG. 10A). The result corroborated the ChIP-seq analysis (FIG. 10B).

### 6.9 Example 9: Pretreatment of PBMCs with Different Concentrations of CC-92480 Enhanced Protein 1 Induced Antibody-Dependent Cellular Cytotoxicity (ADCC)

PBMCs from two healthy donors were pre-incubated for 24 hours or 48 hours in the presence of CC-92480 or a DMSO control. CC-92480 was added at 0.001 nM, 0.01 nM, 0.1 nM, 1 nM and 10 nM concentrations. After pretreatment, PBMCs were incubated for an additional 18 hours or overnight in the presence of H929 target cells with or without the addition of 0.1 nM of Protein 1. Samples were analyzed via flow cytometry for H929 depletion. Specific lysis was calculated as following: (1−((test sample H929 count)/(H929 count in DMSO control without Protein 1)))*100.

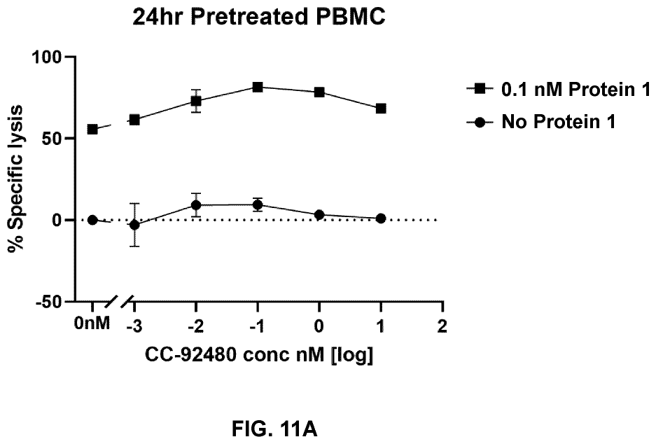

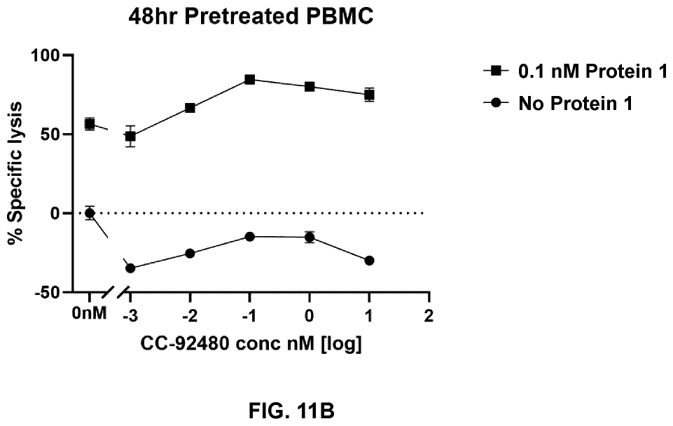

FIGS. 11A-B represent data from one of two different healthy PBMC donors that were pretreated with 0 nM to 10 nM of CC-92480 for either 24 hours (FIG. 11A) or 48 hours (FIG. 11B). After pretreatment, PBMCs were cultured with H929 target cells with or without 0.1 nM Protein 1 overnight. H929 lysis was determined via flow cytometry. Pretreatment of PBMCs with different concentrations of CC-92480 enhanced Protein 1 induced ADCC.

### 6.10 Example 10: Pretreatment of PBMCs with Different Concentrations of Iberdomide (CC-220) Enhanced Protein 1 Induced ADCC

PBMCs from two healthy donors were pre-incubated for 24 hours or 48 hours in the presence of CC-220 or a DMSO control. CC-220 was added at 0.01 nM, 0.1 nM, 1 nM, 10 nM and 100 nM concentrations. After pretreatment, PBMCs were incubated for an additional 18 hours or overnight in the presence of H929 target cells with or without the addition of 0.1 nM of Protein 1. Samples were analyzed via flow cytometry for H929 depletion. Specific lysis was calculated as following: (1−((test sample H929 count)/(H929 count in DMSO control without Protein 1)))*100.

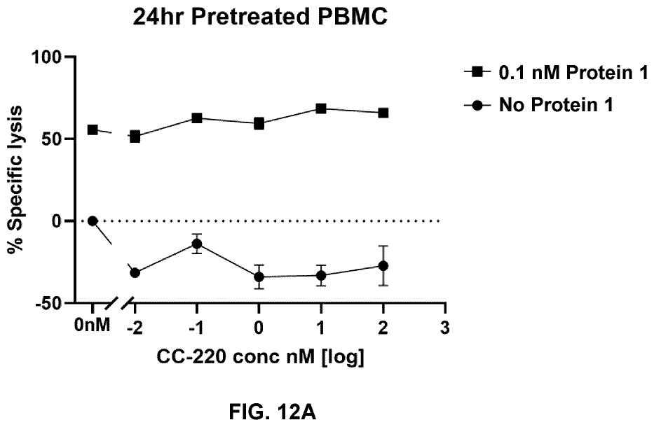

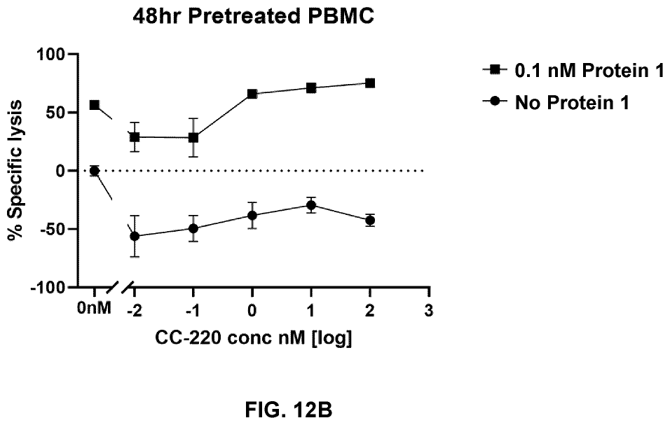

FIGS. 12A-B represent data from one of two different healthy PBMC donors that were pretreated with 0 nM to 10 nM of CC-92480 for either 24 hours (FIG. 12A) or 48 hours (FIG. 12B). After pretreatment, PBMCs were cultured with H929 target cells with or without 0.1 nM Protein 1 overnight. H929 lysis was determined via flow cytometry. Pretreatment of PBMCs with different concentrations of CC-220 enhances Protein 1 induced ADCC.

Table 10 shows that pretreatment of PBMCs with CC-92480 or CC-220 increased Protein 1 mediated ADCC. Data is normalized to % change of sample from the control treated DMSO sample+/−Protein 1. For % lysis without Protein 1, the control sample is PBMC pretreated with DMSO+H929. For % lysis with Protein 1, the control sample is PBMC pretreated with DMSO+H929+0.1 nM of Protein 1.

TABLE 10
Pretreatment of PBMCs with different concentrations of CC-92480 or
Iberdomide (CC-220) increased Protein 1 mediated ADCC

| | Pretreatment | Pretreatment | | % lysis | | % lysis |
|---|---|---|---|---|---|---|
| | concentration | time point | Donor | without | % lysis with | difference |
| Compound | (nM) | (hour) | number | Protein 1 | Protein 1 | with Protein 1 |
| CC-220 | 1 | 24 | 1 | −34.02 | 8.43 | 42.45 |
| CC-220 | 1 | 48 | 1 | −38.19 | 21.68 | 59.87 |
| CC-220 | 1 | 24 | 2 | −28.02 | 46.34 | 74.36 |
| CC-220 | 1 | 48 | 2 | 37.75 | 73.47 | 35.72 |
| CC-220 | 100 | 24 | 1 | −27.15 | 23.24 | 50.39 |
| CC-220 | 100 | 48 | 1 | −42.41 | 43.07 | 85.48 |
| CC-220 | 100 | 24 | 2 | −39.11 | 16.50 | 55.61 |
| CC-220 | 100 | 48 | 2 | 30.14 | 66.54 | 36.40 |
| CC-92480 | 0.1 | 24 | 1 | 9.42 | 58.27 | 48.85 |
| CC-92480 | 0.1 | 48 | 1 | −14.86 | 64.54 | 79.40 |
| CC-92480 | 0.1 | 24 | 2 | −8.82 | 67.82 | 76.64 |
| CC-92480 | 0.1 | 48 | 2 | 45.23 | 82.02 | 36.79 |
| CC-92480 | 10 | 24 | 1 | 1.04 | 28.79 | 27.75 |
| CC-92480 | 10 | 48 | 1 | −29.89 | 42.46 | 72.35 |
| CC-92480 | 10 | 24 | 2 | −10.19 | −43.51 | −33.32 |
| CC-92480 | 10 | 48 | 2 | 6.85 | 59.57 | 52.72 |

### 6.11 Example 11: Pretreatment of PBMCs with Single Concentration of CC-92480 or Iberdomide (CC-220) Enhanced Protein 1 Induced ADCC

PBMCs from a healthy donor were preincubated for 24 hours or 48 hours in the presence of CC-92480 at 0.1 nM or CC-220 (iberdomide) at 1 nM or a DMSO control. After pretreatment, PBMCs were incubated for an additional 18 hours or overnight in the presence of H929 target cells with concentrations of Protein 1 ranging from 0 nM to 10 nM. Samples were analyzed via flow cytometry for H929 depletion. Specific lysis was calculated as following: (1−((test sample H929 count)/(H929 count in DMSO control without Protein 1)))*100.

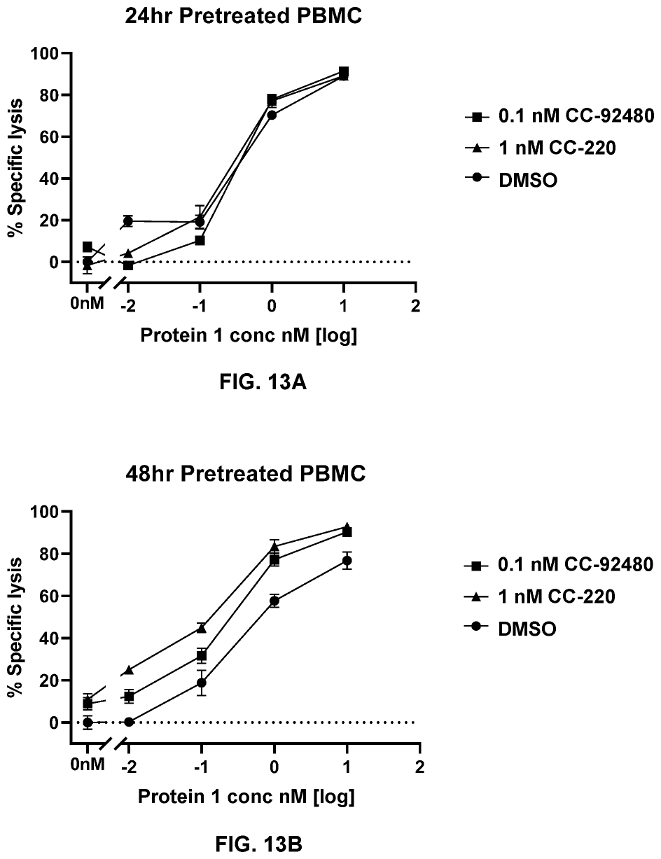

PBMCs were pretreated 0.1 nM of CC-92480 or 1 nM of CC-220 or DMSO control for either 24 hours (FIG. 13A) or 48 hours (FIG. 13B). After pretreatment, PBMCs were cultured with H929 target cells and 0 nM to 10 nM of Protein 1 overnight. H929 lysis was determined via flow cytometry. Pretreatment of PBMCs with single concentration of CC-92480 or CC-220 enhanced Protein 1 induced ADCC.

### 6.12 Example 12: Pretreatment of Both PBMC and 11929 Target Cells with CC-92480 or Iberdomide (CC-220) Enhanced Protein 1 Induced ADCC

PBMCs from a healthy donor were pre-incubated for 48 hours in the presence of CC-92480 or CC-220 (iberdomide) or a DMSO control. Additionally, H929 cells were also pre-incubated separately from the PBMCs for 48 hours with CELMoD. For CC-92480, PBMC and H929 were incubated in 0.1 nM, 1 nM or 10 nM concentrations. For CC-220, cells were incubated in 1 nM or 10 nM concentrations. Cells were also pre-incubated in with a DMSO control. After pretreatment, PBMC and H929 were combined based on their matched treatments and incubated for an additional 18 hours or overnight in the presence of a dose range of Protein 1, 0 nM to 10 nM. Samples were analyzed via flow cytometry for H929 depletion. Specific lysis was calculated as following: (1−((test sample H929 count)/(H929 count in DMSO control without PBMC and without Protein 1)))*100.

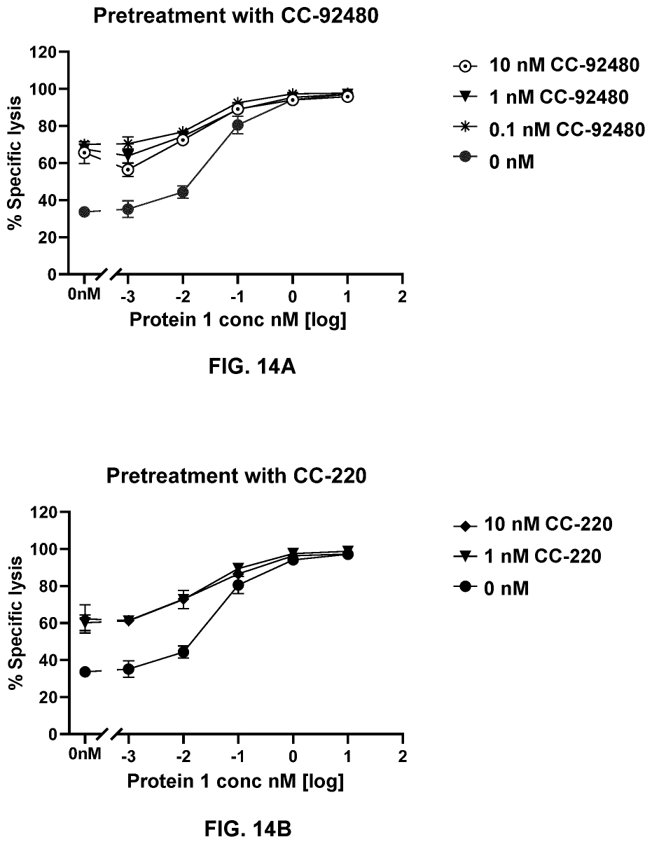

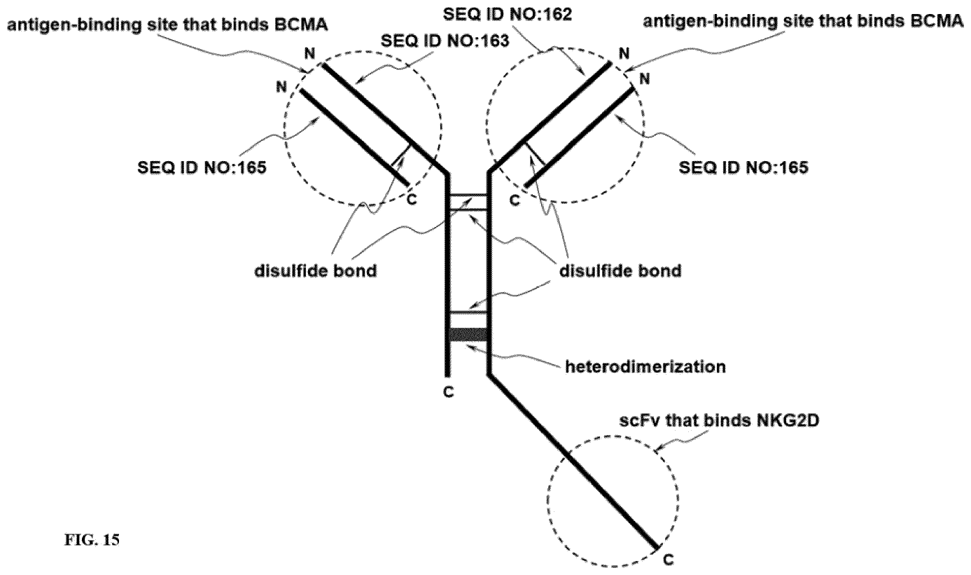

PBMC and H929 cells were pretreated separately with varying doses of CC-92480 (FIG. 14A) or CC-220 (FIG. 14B) or a DMSO control for 48 hours. After pretreatment, PBMCs and H929 target cells were combined and co-cultured overnight with a dose range of Protein 1 at 0 nM to 10 nM. H929 cell depletion was analyzed by flow cytometry. This experiment suggested that pretreating the H929 target cells with CC-92480 or CC-220 makes the target cells more susceptible to lysis. The increased lysis was most notably in the mid-range of the Protein 1 dose curve (0.01-0.1 nM). Pretreatment of both PBMC and H929 target cells with CC-92480 or CC-220 (iberdomide) enhanced Protein 1 induced ADCC.

### EQUIVALENTS

The methods and pharmaceutical compositions disclosed herein are not to be limited in scope by the specific embodiments described herein. Indeed, various modifications of methods and pharmaceutical compositions in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

TABLE OF SEQUENCES

| SEQ | | |
|---|---|---|
| ID | | |
| NO. | Description | Sequence |
| 1 | Heavy chain variable region of | QVOLQQWGAGLLKPSETLSLTCAVYGGSFSGYY |
| | clone ADI-27705 | WSWIRQPPGKGLEWIGEIDHSGSTNYNPSLKSR |
| | | VTISVDTSKNQFSLKLSSVTAADTAVYYCARAR |
| | | GPWSFDPWGQGTLVTVSS |
| 2 | Light chain variable region of | DIQMTQSPSTLSASVGDRVTITCRASQSISSWL |
| | clone ADI-27705 | AWYQQKPGKAPKLLIYKASSLESGVPSRFSGSG |
| | | SGTEFTLTISSLQPDDFATYYCQQYNSYPITFG |
| | | GGTKVEIK |
| 3 | CDR1 of the heavy chain | GSFSGYYWS |
| | variable region of clone ADI- | |
| | 27705 | |
| 4 | CDR2 of the heavy chain | EIDHSGSTNYNPSLKS |
| | variable region of clone ADI- | |
| | 27705 | |
| 5 | CDR3 of the heavy chain | ARARGPWSFDP |
| | variable region of clone ADI- | |
| | 27705 | |
| 6 | Heavy chain variable region of | QVQLQQWGAGLLKPSETLSLTCAVYGGSFSGYY |
| | clone ADI-27724 | WSWIRQPPGKGLEWIGEIDHSGSTNYNPSLKSR |
| | | VTISVDTSKNQFSLKLSSVTAADTAVYYCARAR |
| | | GPWSFDPWGQGTLVTVSS |
| 7 | Light chain variable region of | EIVLTQSPGTLSLSPGERATLSCRASQSVSSSY |
| | clone ADI-27724 | LAWYQQKPGQAPRLLIYGASSRATGIPDRFSGS |
| | | GSGTDFTLTISRLEPEDFAVYYCQQYGSSPITF |
| | | GGGTKVEIK |
| 8 | Heavy chain variable region of | QVOLQQWGAGLLKPSETLSLTCAVYGGSFSGYY |
| | clone ADI-27740 (A40) | WSWIRQPPGKGLEWIGEIDHSGSTNYNPSLKSR |
| | | VTISVDTSKNQFSLKLSSVTAADTAVYYCARAR |
| | | GPWSFDPWGQGTLVTVSS |
| 9 | Light chain variable region of | DIQMTQSPSTLSASVGDRVTITCRASQSIGSWL |
| | clone ADI-27740 (A40) | AWYQQKPGKAPKLLIYKASSLESGVPSRFSGSG |
| | | SGTEFTLTISSLQPDDFATYYCQQYHSFYTFGG |
| | | GTKVEIK |
| 10 | Heavy chain variable region of | QVQLQQWGAGLLKPSETLSLTCAVYGGSFSGYY |
| | clone ADI-27741 | WSWIRQPPGKGLEWIGEIDHSGSTNYNPSLKSR |
| | | VTISVDTSKNQFSLKLSSVTAADTAVYYCARAR |
| | | GPWSFDPWGQGTLVTVSS |
| 11 | Light chain variable region of | DIQMTQSPSTLSASVGDRVTITCRASQSIGSWL |
| | clone ADI-27741 | AWYQQKPGKAPKLLIYKASSLESGVPSRFSGSG |
| | | SGTEFTLTISSLQPDDFATYYCQQSNSYYTFGG |
| | | GTKVEIK |
| 12 | Heavy chain variable region of | QVOLQQWGAGLLKPSETLSLTCAVYGGSFSGYY |
| | clone ADI-27743 | WSWIRQPPGKGLEWIGEIDHSGSTNYNPSLKSR |
| | | VTISVDTSKNQFSLKLSSVTAADTAVYYCARAR |
| | | GPWSFDPWGQGTLVTVSS |
| 13 | Light chain variable region of | DIQMTQSPSTLSASVGDRVTITCRASQSISSWL |
| | clone ADI-27743 | AWYQQKPGKAPKLLIYKASSLESGVPSRFSGSG |
| | | SGTEFTLTISSLQPDDFATYYCQQYNSYPTFGG |
| | | GTKVEIK |
| 14 | Heavy chain variable region of | QVOLQQWGAGLLKPSETLSLTCAVYGGSFSGYY |
| | clone ADI-28153 | WSWIRQPPGKGLEWIGEIDHSGSTNYNPSLKSR |
| | | VTISVDTSKNQFSLKLSSVTAADTAVYYCARAR |
| | | GPWGFDPWGQGTLVTVSS |
| 15 | Light chain variable region of | ELQMTQSPSSLSASVGDRVTITCRTSQSISSYL |
| | clone ADI-28153 | NWYQQKPGQPPKLLIYWASTRESGVPDRFSGSG |
| | | SGTDFTLTISSLOPEDSATYYCQQSYDIPYTFG |
| | | QGTKLEIK |
| 16 | Heavy chain variable region of | QVQLQQWGAGLLKPSETLSLTCAVYGGSFSGYY |
| | clone ADI-28226 | WSWIRQPPGKGLEWIGEIDHSGSTNYNPSLKSR |
| | (C26) | VTISVDTSKNQFSLKLSSVTAADTAVYYCARAR |
| | | GPWSFDPWGQGTLVTVSS |
| 17 | Light chain variable region of | DIQMTOSPSTLSASVGDRVTITCRASQSISSWL |
| | clone ADI-28226 | AWYQQKPGKAPKLLIYKASSLESGVPSRFSGSG |
| | (C26) | SGTEFTLTISSLQPDDFATYYCQQYGSFPITFG |
| | | GGTKVEIK |
| 18 | Heavy chain variable region of | QVQLQQWGAGLLKPSETLSLTCAVYGGSFSGYY |
| | clone ADI-28154 | WSWIRQPPGKGLEWIGEIDHSGSTNYNPSLKSR |
| | | VTISVDTSKNQFSLKLSSVTAADTAVYYCARAR |
| | | GPWSFDPWGQGTLVTVSS |
| 19 | Light chain variable region of | DIQMTQSPSTLSASVGDRVTITCRASQSISSWL |
| | clone ADI-28154 | AWYQQKPGKAPKLLIYKASSLESGVPSRFSGSG |
| | | SGTDFTLTISSLQPDDFATYYCQQSKEVPWTFG |
| | | QGTKVEIK |
| 20 | Heavy chain variable region of | QVOLQQWGAGLLKPSETLSLTCAVYGGSFSGYY |
| | clone ADI-29399 | WSWIRQPPGKGLEWIGEIDHSGSTNYNPSLKSR |
| | | VTISVDTSKNQFSLKLSSVTAADTAVYYCARAR |
| | | GPWSFDPWGQGTLVTVSS |
| 21 | Light chain variable region of | DIQMTQSPSTLSASVGDRVTITCRASQSISSWL |
| | clone ADI-29399 | AWYQQKPGKAPKLLIYKASSLESGVPSRFSGSG |
| | | SGTEFTLTISSLQPDDFATYYCQQYNSFPTFGG |
| | | GTKVEIK |
| 22 | Heavy chain variable region of | QVQLQQWGAGLLKPSETLSLTCAVYGGSFSGYY |
| | clone ADI-29401 | WSWIRQPPGKGLEWIGEIDHSGSTNYNPSLKSR |
| | | VTISVDTSKNQFSLKLSSVTAADTAVYYCARAR |
| | | GPWSFDPWGQGTLVTVSS |
| 23 | Light chain variable region of | DIQMTQSPSTLSASVGDRVTITCRASQSIGSWL |
| | clone ADI-29401 | AWYQQKPGKAPKLLIYKASSLESGVPSRFSGSG |
| | | SGTEFTLTISSLQPDDFATYYCQQYDIYPTFGG |
| | | GTKVEIK |
| 24 | Heavy chain variable region of | QVQLQQWGAGLLKPSETLSLTCAVYGGSFSGYY |
| | clone ADI-29403 | WSWIRQPPGKGLEWIGEIDHSGSTNYNPSLKSR |
| | | VTISVDTSKNQFSLKLSSVTAADTAVYYCARAR |
| | | GPWSFDPWGQGTLVTVSS |
| 25 | Light chain variable region of | DIQMTQSPSTLSASVGDRVTITCRASQSISSWL |
| | clone ADI-29403 | AWYQQKPGKAPKLLIYKASSLESGVPSRFSGSG |
| | | SGTEFTLTISSLQPDDFATYYCQQYDSYPTFGG |
| | | GTKVEIK |
| 26 | Heavy chain variable region of | QVQLQQWGAGLLKPSETLSLTCAVYGGSFSGYY |
| | clone ADI-29405 | WSWIRQPPGKGLEWIGEIDHSGSTNYNPSLKSR |
| | | VTISVDTSKNQFSLKLSSVTAADTAVYYCARAR |
| | | GPWSFDPWGQGTLVTVSS |
| 27 | Light chain variable region of | DIQMTQSPSTLSASVGDRVTITCRASQSISSWL |
| | clone ADI-29405 | AWYQQKPGKAPKLLIYKASSLESGVPSRFSGSG |
| | | SGTEFTLTISSLQPDDFATYYCQQYGSFPTFGG |
| | | GTKVEIK |
| 28 | Heavy chain variable region of | QVQLQQWGAGLLKPSETLSLTCAVYGGSFSGYY |
| | clone ADI-29407 | WSWIRQPPGKGLEWIGEIDHSGSTNYNPSLKSR |
| | | VTISVDTSKNQFSLKLSSVTAADTAVYYCARAR |
| | | GPWSFDPWGQGTLVTVSS |
| 29 | Light chain variable region of | DIQMTQSPSTLSASVGDRVTITCRASQSISSWL |
| | clone ADI-29407 | AWYQQKPGKAPKLLIYKASSLESGVPSRFSGSG |
| | | SGTEFTLTISSLQPDDFATYYCQQYQSFPTFGG |
| | | GTKVEIK |
| 30 | Heavy chain variable region of | QVQLQQWGAGLLKPSETLSLTCAVYGGSFSGYY |
| | clone ADI-29419 | WSWIRQPPGKGLEWIGEIDHSGSTNYNPSLKSR |
| | | VTISVDTSKNQFSLKLSSVTAADTAVYYCARAR |
| | | GPWSFDPWGQGTLVTVSS |
| 31 | Light chain variable region of | DIQMTQSPSTLSASVGDRVTITCRASQSISSWL |
| | clone ADI-29419 | AWYQQKPGKAPKLLIYKASSLESGVPSRFSGSG |
| | | SGTEFTLTISSLQPDDFATYYCQQYSSFSTFGG |
| | | GTKVEIK |
| 32 | Heavy chain variable region of | QVQLQQWGAGLLKPSETLSLTCAVYGGSFSGYY |
| | clone ADI-29421 | WSWIRQPPGKGLEWIGEIDHSGSTNYNPSLKSR |
| | | VTISVDTSKNQFSLKLSSVTAADTAVYYCARAR |
| | | GPWSFDPWGQGTLVTVSS |
| 33 | Light chain variable region of | DIQMTQSPSTLSASVGDRVTITCRASQSISSWL |
| | clone ADI-29421 | AWYQQKPGKAPKLLIYKASSLESGVPSRFSGSG |
| | | SGTEFTLTISSLQPDDFATYYCQQYESYSTFGG |
| | | GTKVEIK |
| 34 | Heavy chain variable region of | QVQLQQWGAGLLKPSETLSLTCAVYGGSFSGYY |
| | clone ADI-29424 | WSWIRQPPGKGLEWIGEIDHSGSTNYNPSLKSR |
| | | VTISVDTSKNQFSLKLSSVTAADTAVYYCARAR |
| | | GPWSFDPWGQGTLVTVSS |
| 35 | Light chain variable region of | DIQMTQSPSTLSASVGDRVTITCRASQSISSWL |
| | clone ADI-29424 | AWYQQKPGKAPKLLIYKASSLESGVPSRFSGSG |
| | | SGTEFTLTISSLQPDDFATYYCQQYDSFITFGG |
| | | GTKVEIK |
| 36 | Heavy chain variable region of | QVQLQQWGAGLLKPSETLSLTCAVYGGSFSGYY |
| | clone ADI-29425 | WSWIRQPPGKGLEWIGEIDHSGSTNYNPSLKSR |
| | | VTISVDTSKNQFSLKLSSVTAADTAVYYCARAR |
| | | GPWSFDPWGQGTLVTVSS |
| 37 | Light chain variable region of | DIQMTQSPSTLSASVGDRVTITCRASQSISSWL |
| | clone ADI-29425 | AWYQQKPGKAPKLLIYKASSLESGVPSRFSGSG |
| | | SGTEFTLTISSLQPDDFATYYCQQYQSYPTFGG |
| | | GTKVEIK |
| 38 | Heavy chain variable region of | QVQLQQWGAGLLKPSETLSLTCAVYGGSFSGYY |
| | clone ADI-29426 | WSWIRQPPGKGLEWIGEIDHSGSTNYNPSLKSR |
| | | VTISVDTSKNQFSLKLSSVTAADTAVYYCARAR |
| | | GPWSFDPWGQGTLVTVSS |
| 39 | Light chain variable region of | DIQMTQSPSTLSASVGDRVTITCRASQSIGSWL |
| | clone ADI-29426 | AWYQQKPGKAPKLLIYKASSLESGVPSRFSGSG |
| | | SGTEFTLTISSLQPDDFATYYCQQYHSFPTFGG |
| | | GTKVEIK |
| 40 | Heavy chain variable region of | QVQLQQWGAGLLKPSETLSLTCAVYGGSFSGYY |
| | clone ADI-29429 | WSWIRQPPGKGLEWIGEIDHSGSTNYNPSLKSR |
| | | VTISVDTSKNQFSLKLSSVTAADTAVYYCARAR |
| | | GPWSFDPWGQGTLVTVSS |
| 41 | Light chain variable region of | DIQMTQSPSTLSASVGDRVTITCRASQSIGSWL |
| | clone ADI-29429 | AWYQQKPGKAPKLLIYKASSLESGVPSRFSGSG |
| | | SGTEFTLTISSLQPDDFATYYCQQYELYSYTFG |
| | | GGTKVEIK |
| 42 | Heavy chain variable region of | QVOLQQWGAGLLKPSETLSLTCAVYGGSFSGYY |
| | clone ADI-29447 | WSWIRQPPGKGLEWIGEIDHSGSTNYNPSLKSR |
| | (F47) | VTISVDTSKNQFSLKLSSVTAADTAVYYCARAR |
| | | GPWSFDPWGQGTLVTVSS |
| 43 | Light chain variable region of | DIQMTQSPSTLSASVGDRVTITCRASQSISSWL |
| | clone ADI-29447 | AWYQQKPGKAPKLLIYKASSLESGVPSRFSGSG |
| | (F47) | SGTEFTLTISSLQPDDFATYYCQQYDTFITFGG |
| | | GTKVEIK |
| 44 | Heavy chain variable region of | QVQLVQSGAEVKKPGSSVKVSCKASGGTFSSYA |
| | clone ADI-27727 | ISWVRQAPGQGLEWMGGIIPIFGTANYAQKFQG |
| | | RVTITADESTSTAYMELSSLRSEDTAVYYCARG |
| | | DSSIRHAYYYYGMDVWGQGTTVTVSS |
| 45 | CDR1 of the heavy chain | GTFSSYAIS |
| | variable region of clone ADI- | |
| | 27727 (non-Kabat) | |
| 46 | CDR2 of the heavy chain | GIIPIFGTANYAQKFQG |
| | variable region of clone ADI- | |
| | 27727 | |
| 47 | CDR3 of the heavy chain | ARGDSSIRHAYYYYGMDV |
| | variable region of clone ADI- | |
| | 27727 (non-Kabat) | |
| 48 | Light chain variable region of | DIVMTQSPDSLAVSLGERATINCKSSQSVLYSS |
| | clone ADI-27727 | NNKNYLAWYQQKPGQPPKLLIYWASTRESGVPD |
| | | RFSGSGSGTDFTLTISSLQAEDVAVYYCQQYYS |
| | | TPITFGGGTKVEIK |
| 49 | CDR1 of the light chain | KSSQSVLYSSNNKNYLA |
| | variable region of clone ADI- | |
| | 27727 | |
| 50 | CDR2 of the light chain | WASTRES |
| | variable region of clone ADI- | |
| | 27727 | |
| 51 | CDR3 of the light chain | QQYYSTPIT |
| | variable region of clone ADI- | |
| | 27727 | |
| 52 | Heavy chain variable region of | QLQLQESGPGLVKPSETLSLTCTVSGGSISSSS |
| | clone ADI-29443 (F43) | YYWGWIRQPPGKGLEWIGSIYYSGSTYYNPSLK |
| | | SRVTISVDTSKNQFSLKLSSVTAADTAVYYCAR |
| | | GSDRFHPYFDYWGQGTLVTVSS |
| 53 | CDR1 of the heavy chain | GSISSSSYYWG |
| | variable region of clone ADI- | |
| | 29443 (F43) (non-Kabat) | |
| 54 | CDR2 of the heavy chain | SIYYSGSTYYNPSLKS |
| | variable region of clone ADI- | |
| | 29443 (F43) | |
| 55 | CDR3 of the heavy chain | ARGSDRFHPYFDY |
| | variable region of clone ADI- | |
| | 29443 (F43) (non-Kabat) | |
| 56 | Light chain variable region of | EIVLTQSPATLSLSPGERATLSCRASQSVSRYL |
| | clone ADI-29443 (F43) | AWYQQKPGQAPRLLIYDASNRATGIPARFSGSG |
| | | SGTDFTLTISSLEPEDFAVYYCQQFDTWPPTFG |
| | | GGTKVEIK |
| 57 | CDR1 of the light chain | RASQSVSRYLA |
| | variable region of clone ADI- | |
| | 29443 (F43) | |
| 58 | CDR2 of the light chain | DASNRAT |
| | variable region of clone ADI- | |
| | 29443 (F43) | |
| 59 | CDR3 of the light chain | QQFDTWPPT |
| | variable region of clone ADI- | |
| | 29443 (F43) | |
| 60 | Heavy chain variable region of | QVQLQQWGAGLLKPSETLSLTCAVYGGSFSGYY |
| | clone ADI-29404 (F04) | WSWIRQPPGKGLEWIGEIDHSGSTNYNPSLKSR |
| | | VTISVDTSKNQFSLKLSSVTAADTAVYYCARAR |
| | | GPWSFDPWGQGTLVTVSS |
| 61 | Light chain variable region of | DIQMTQSPSTLSASVGDRVTITCRASQSISSWL |
| | clone ADI-29404 (F04) | AWYQQKPGKAPKLLIYKASSLESGVPSRFSGSG |
| | | SGTEFTLTISSLQPDDFATYYCEQYDSYPTFGG |
| | | GTKVEIK |
| 62 | Heavy chain variable region of | QVQLVQSGAEVKKPGSSVKVSCKASGGTFSSYA |
| | clone ADI-28200 | ISWVRQAPGOGLEWMGGIIPIFGTANYAQKFQG |
| | | RVTITADESTSTAYMELSSLRSEDTAVYYCARR |
| | | GRKASGSFYYYYGMDVWGQGTTVTVSS |
| 63 | CDR1 of the heavy chain | GTFSSYAIS |
| | variable region of clone ADI- | |
| | 28200 (non-Kabat) | |
| 64 | CDR2 of the heavy chain | GIIPIFGTANYAQKFQG |
| | variable region of clone ADI- | |
| | 28200 | |
| 65 | CDR3 of the heavy chain | ARRGRKASGSFYYYYGMDV |
| | variable region of clone ADI- | |
| | 28200 | |
| 66 | Light chain variable region of | DIVMTQSPDSLAVSLGERATINCESSQSLLNSG |
| | clone ADI-28200 | NQKNYLTWYQQKPGQPPKPLIYWASTRESGVPD |
| | | RFSGSGSGTDFTLTISSLQAEDVAVYYCONDYS |
| | | YPYTFGQGTKLEIK |
| 67 | CDR1 of the light chain | ESSQSLLNSGNQKNYLT |
| | variable region of clone ADI- | |
| | 28200 | |
| 68 | CDR2 of the light chain | WASTRES |
| | variable region of clone ADI- | |
| | 28200 | |
| 69 | CDR3 of the light chain | QNDYSYPYT |
| | variable region of clone ADI- | |
| | 28200 | |
| 70 | Heavy chain variable region of | QVQLVQSGAEVKKPGASVKVSCKASGYTFTSYY |
| | clone ADI-29379 (E79) | MHWVRQAPGOGLEWMGIINPSGGSTSYAQKFQG |
| | | RVTMTRDTSTSTVYMELSSLRSEDTAVYYCARG |
| | | APNYGDTTHDYYYMDVWGKGTTVTVSS |
| 71 | CDR1 of the heavy chain | YTFTSYYMH |
| | variable region of clone ADI- | |
| | 29379 (E79) (non-Kabat) | |
| 72 | CDR2 of the heavy chain | IINPSGGSTSYAQKFQG |
| | variable region of clone ADI- | |
| | 29379 (E79) | |
| 73 | CDR3 of the heavy chain | ARGAPNYGDTTHDYYYMDV |
| | variable region of clone ADI- | |
| | 29379 (E79) (non-Kabat) | |
| 74 | Light chain variable region of | EIVMTQSPATLSVSPGERATLSCRASQSVSSNL |
| | clone ADI-29379 (E79) | AWYQQKPGQAPRLLIYGASTRATGIPARFSGSG |
| | | SGTEFTLTISSLQSEDFAVYYCQQYDDWPFTFG |
| | | GGTKVEIK |
| 75 | CDR1 of the light chain | RASQSVSSNLA |
| | variable region of clone ADI- | |
| | 29379 (E79) | |
| 76 | CDR2 of the light chain | GASTRAT |
| | variable region of clone ADI- | |
| | 29379 (E79) | |
| 77 | CDR3 of the light chain | QQYDDWPFT |
| | variable region of clone ADI- | |
| | 29379 (E79) | |
| 78 | Heavy chain variable region of | QVQLVQSGAEVKKPGASVKVSCKASGYTFTGYY |
| | clone ADI-29463 (F63) | MHWVRQAPGQGLEWMGWINPNSGGTNYAQKFQG |
| | | RVTMTRDTSISTAYMELSRLRSDDTAVYYCARD |
| | | TGEYYDTDDHGMDVWGQGTTVTVSS |
| 79 | CDR1 of the heavy chain | YTFTGYYMH |
| | variable region of clone ADI- | |
| | 29463 (F63) (non-Kabat) | |
| 80 | CDR2 of the heavy chain | WINPNSGGTNYAQKFQG |
| | variable region of clone ADI- | |
| | 29463 (F63) | |
| 81 | CDR3 of the heavy chain | ARDTGEYYDTDDHGMDV |
| | variable region of clone ADI- | |
| | 29463 (F63) (non-Kabat) | |
| 82 | Light chain variable region of | EIVLTQSPGTLSLSPGERATLSCRASQSVSSNL |
| | clone ADI-29463 (F63) | AWYQQKPGQAPRLLIYGASTRATGIPARFSGSG |
| | | SGTEFTLTISSLQSEDFAVYYCQQDDYWPPTFG |
| | | GGTKVEIK |
| 83 | CDR1 of the light chain | RASQSVSSNLA |
| | variable region of clone ADI- | |
| | 29463 (F63) | |
| 84 | CDR2 of the light chain | GASTRAT |
| | variable region of clone ADI- | |
| | 29463 (F63) | |
| 85 | CDR3 of the light chain | QQDDYWPPT |
| | variable region of clone ADI- | |
| | 29463 (F63) | |
| 86 | Heavy chain variable region of | EVQLLESGGGLVQPGGSLRLSCAASGFTFSSYA |
| | clone ADI-27744 (A44) | MSWVRQAPGKGLEWVSAISGSGGSTYYADSVKG |
| | | RFTISRDNSKNTLYLQMNSLRAEDTAVYYCAKD |
| | | GGYYDSGAGDYWGQGTLVTVSS |
| 87 | CDR1 of the heavy chain | FTFSSYAMS |
| | variable region of clone ADI- | |
| | 27744 (A44) (non-Kabat) | |
| 88 | CDR2 of the heavy chain | AISGSGGSTYYADSVKG |
| | variable region of clone ADI- | |
| | 27744 (A44) | |
| 89 | CDR3 of the heavy chain | AKDGGYYDSGAGDY |
| | variable region of clone ADI- | |
| | 27744 (A44) (non-Kabat) | |
| 90 | Light chain variable region of | DIQMTQSPSSVSASVGDRVTITCRASQGIDSWL |
| | clone ADI-27744 (A44) | AWYQQKPGKAPKLLIYAASSLQSGVPSRFSGSG |
| | | SGTDFTLTISSLOPEDFATYYCQQGVSYPRTFG |
| | | GGTKVEIK |
| 91 | CDR1 of the light chain | RASQGIDSWLA |
| | variable region of clone ADI- | |
| | 27744 (A44) | |
| 92 | CDR2 of the light chain | AASSLQS |
| | variable region of clone ADI- | |
| | 27744 (A44) | |
| 93 | CDR3 of the light chain | QQGVSYPRT |
| | variable region of clone ADI- | |
| | 27744 (A44) | |
| 94 | Heavy chain variable region of | EVQLVESGGGLVKPGGSLRLSCAASGFTFSSYS |
| | clone ADI-27749 (A49) | MNWVRQAPGKGLEWVSSISSSSSYIYYADSVKG |
| | | RFTISRDNAKNSLYLQMNSLRAEDTAVYYCARG |
| | | APMGAAAGWFDPWGQGTLVTVSS |
| 95 | CDR1 of the heavy chain | FTFSSYSMN |
| | variable region of clone ADI- | |
| | 27749 (A49), A49MI, A49MQ, | |
| | A49ML, A49MF, A49MV, or | |
| | A49-consensus (non-Kabat) | |
| 96 | CDR2 of the heavy chain | SISSSSSYIYYADSVKG |
| | variable region of clone ADI- | |
| | 27749 (A49), A49MI, A49MQ, | |
| | A49ML, A49MF, A49MV, or | |
| | A49-consensus (non-Kabat) | |
| 97 | CDR3 of the heavy chain | ARGAPMGAAAGWFDP |
| | variable region of clone ADI- | |
| | 27749 (A49) (non-Kabat) | |
| 98 | Light chain variable region of | DIQMTQSPSSVSASVGDRVTITCRASQGISSWL |
| | clone ADI-27749 (A49), A49MI, | AWYQQKPGKAPKLLIYAASSLQSGVPSRFSGSG |
| | A49MQ, A49ML, A49MF, A49MV, or | SGTDFTLTISSLQPEDFATYYCQQGVSFPRTFG |
| | A49-consensus | GGTKVEIK |
| 99 | CDR1 of the light chain | RASQGISSWLA |
| | variable region of clone ADI- | |
| | 27749 (A49), A49MI, A49MQ, | |
| | A49ML, A49MF, A49MV, or | |
| | A49-consensus | |
| 100 | CDR2 of the light chain | AASSLQS |
| | variable region of clone ADI- | |
| | 27749 (A49), A49MI, A49MQ, | |
| | A49ML, A49MF, A49MV, or | |
| | A49-consensus | |
| 101 | CDR3 of the light chain | QQGVSFPRT |
| | variable region of clone ADI- | |
| | 27749 (A49), A49MI, A49MQ, | |
| | A49ML, A49MF, A49MV, or | |
| | A49-consensus | |
| 102 | Heavy chain variable region of | QVQLVQSGAEVKKPGASVKVSCKASGYTFTSYY |
| | clone ADI-29378 (E78) | MHWVRQAPGOGLEWMGIINPSGGSTSYAQKFQG |
| | | RVTMTRDTSTSTVYMELSSLRSEDTAVYYCARE |
| | | GAGFAYGMDYYYMDVWGKGTTVTVSS |
| 103 | CDR1 of the heavy chain | YTFTSYYMH |
| | variable region of clone ADI- | |
| | 29378 (E78) (non-Kabat) | |
| 104 | CDR2 of the heavy chain | IINPSGGSTSYAQKFQG |
| | variable region of clone ADI- | |
| | 29378 (E78) | |
| 105 | CDR3 of the heavy chain | AREGAGFAYGMDYYYMDV |
| | variable region of clone ADI- | |
| | 29378 (E78) | |
| 106 | Light chain variable region of | EIVLTQSPATLSLSPGERATLSCRASQSVSSYL |
| | clone ADI-29378 (E78) | AWYQQKPGQAPRLLIYDASNRATGIPARFSGSG |
| | | SGTDFTLTISSLEPEDFAVYYCQQSDNWPFTFG |
| | | GGTKVEIK |
| 107 | CDR1 of the light chain | RASQSVSSYLA |
| | variable region of clone ADI- | |
| | 29378 (E78) | |
| 108 | CDR2 of the light chain | DASNRAT |
| | variable region of clone ADI- | |
| | 29378 (E78) | |
| 109 | CDR3 of the light chain | QQSDNWPFT |
| | variable region of clone ADI- | |
| | 29378 (E78) | |
| 110 | Heavy chain variable domain of | QVQLVESGGGLVKPGGSLRLSCAASGFTFSSYG |
| | an antigen-binding site that | MHWVRQAPGKGLEWVAFIRYDGSNKYYADSVKG |
| | can bind to NKG2D | RFTISRDNSKNTLYLQMNSLRAEDTAVYYCAKD |
| | | RGLGDGTYFDYWGQGTTVTVSS |
| 111 | Light chain variable domain of | QSALTQPASVSGSPGQSITISCSGSSSNIGNNA |
| | an antigen-binding site that | VNWYQQLPGKAPKLLIYYDDLLPSGVSDRFSGS |
| | can bind to NKG2D | KSGTSAFLAISGLQSEDEADYYCAAWDDSLNGP |
| | | VFGGGTKLTVL |
| 112 | Heavy chain variable domain of | QVHLQESGPGLVKPSETLSLTCTVSDDSISSYY |
| | an antigen-binding site that | WSWIRQPPGKGLEWIGHISYSGSANYNPSLKSR |
| | can bind to NKG2D | VTISVDTSKNQFSLKLSSVTAADTAVYYCANWD |
| | | DAFNIWGQGTMVTVSS |
| 113 | Light chain variable domain of | EIVLTQSPGTLSLSPGERATLSCRASQSVSSSY |
| | an antigen-binding site that | LAWYQQKPGQAPRLLIYGASSRATGIPDRFSGS |
| | can bind to NKG2D | GSGTDFTLTISRLEPEDFAVYYCQQYGSSPWTF |
| | | GQGTKVEIK |
| 114 | Heavy chain variable domain of | QVQLVQSGAEVKKPGASVKVSCKASGYSFPDYY |
| | clone 1 (US14/776,649) | INWVROAPGOGLEWMGWIYFASGNSEYNQKFTG |
| | | RVTMTRDTSSSTAYMELSSLRSEDTAVYFCASL |
| | | YDYDWYFDVWGQGTMVTVSS |
| 115 | CDR1 of the heavy chain | DYYIN |
| | variable domain of clone 1 | |
| | (US14/776,649) | |
| 116 | CDR2 of the heavy chain | WIYFASGNSEYNQKFTG |
| | variable domain of clone 1 | |
| | (US14/776,649) | |
| 117 | CDR3 of the heavy chain | LYDYDWYFDV |
| | variable domain of clone 1 | |
| | (US14/776,649) | |
| 118 | Light chain variable domain of | DIVMTQTPLSLSVTPGEPASISCKSSQSLVHSN |
| | clone 1 (US14/776,649) | GNTYLHWYLQKPGQSPQLLIYKVSNRFSGVPDR |
| | | FSGSGSGADFTLKISRVEAEDVGVYYCAETSHV |
| | | PWTFGQGTKLEIK |
| 119 | Light chain variable domain of | DIVMTQTPLSLSVTPGQPASISCKSSQSLVHSN |
| | clone 1 (US14/776,649) | GNTYLHWYLQKPGQSPQLLIYKVSNRFSGVPDR |
| | | FSGSGSGTDFTLKISRVEAEDVGIYYCSQSSIY |
| | | PWTFGQGTKLEIK |
| 120 | CDR1 of the light chain | KSSQSLVHSNGNTYLH |
| | variable domain of clone 1 | |
| | (US14/776,649) | |
| 121 | CDR2 of the light chain | KVSNRFS |
| | variable domain of clone 1 | |
| | (US14/776,649) | |
| 122 | CDR3 of the light chain | AETSHVPWT |
| | variable domain of clone 1 | |
| | (US14/776,649) | |
| 123 | CDR3 of the light chain | SQSSIYPWT |
| | variable domain of clone 1 | |
| | (US14/776,649) | |
| 124 | Heavy chain variable domain of | QIQLVQSGPELKKPGETVKISCKASGYTFTDYS |
| | clone 2 (PCT/US15/64269) | INWVKRAPGKGLKWMGWINTETREPAYAYDFRG |
| | | RFAFSLETSASTAYLQINNLKYEDTATYFCALD |
| | | YSYAMDYWGQGTSVTVSS |
| 125 | CDR1 of the heavy chain | DYSIN |
| | variable domain of clone 2 | |
| | (PCT/US15/64269) | |
| 126 | CDR2 of the heavy chain | WINTETREPAYAYDFR |
| | variable domain of clone 2 | |
| | (PCT/US15/64269) | |
| 127 | CDR3 of the heavy chain | DYSYAMDY |
| | variable domain of clone 2 | |
| | (PCT/US15/64269) | |
| 128 | Light chain variable domain of | DIVLTQSPPSLAMSLGKRATISCRASESVTILG |
| | clone 2 (PCT/US15/64269) | SHLIHWYQQKPGQPPTLLIQLASNVQTGVPARF |
| | | SGSGSRTDFTLTIDPVEEDDVAVYYCLQSRTIP |
| | | RTFGGGTKLEIK |
| 129 | CDR1 of the light chain | RASESVTILGSHLIH |
| | variable domain of clone 2 | |
| | (PCT/US15/64269) | |
| 130 | CDR2 of the light chain | LASNVQT |
| | variable domain of clone 2 | |
| | (PCT/US15/64269) | |
| 131 | CDR3 of the light chain | LQSRTIPRT |
| | variable domain of clone 2 | |
| | (PCT/US15/64269) | |
| 132 | Heavy chain variable domain of | QVQLVQSGAEVKKPGSSVKVSCKASGGTFSNYW |
| | clone 3 (US14/122,391) | MHWVRQAPGQGLEWMGATYRGHSDTYYNQKFKG |
| | | RVTITADKSTSTAYMELSSLRSEDTAVYYCARG |
| | | ATYNGYDVLDNWGQGTLVTVSS |
| 133 | CDR1 of the heavy chain | NYWMH |
| | variable domain of clone 3 | |
| | (US14/122,391) | |
| 134 | CDR2 of the heavy chain | ATYRGHSDTYYNQKFKG |
| | variable domain of clone 3 | |
| | (US14/122,391) | |
| 135 | CDR3 of the heavy chain | GAIYNGYDVLDN |
| | variable domain of clone 3 | |
| | (US14/122,391) | |
| 136 | Light chain variable domain of | DIQMTQSPSSLSASVGDRVTITCSASQDISNYL |
| | clone 3 (US14/122,391) | NWYQQKPGKAPKLLIYYTSNLHSGVPSRFSGSG |
| | | SGTDFTLTISSLQPEDFATYYCQQYRKLPWTFG |
| | | QGTKLEIKR |
| 137 | CDR1 of the light chain | SASQDISNYLN |
| | variable domain of clone 3 | |
| | (US14/122,391) | |
| 138 | CDR2 of the light chain | YTSNLHS |
| | variable domain of clone 3 | |
| | (US14/122,391) | |
| 139 | CDR3 of the light chain | QQYRKLPWT |
| | variable domain of clone 3 | |
| | (US14/122,391) | |
| 140 | Heavy chain variable domain of | QLQLQESGPGLVKPSETLSLTCTVSGGSISSSS |
| | clone 4 (US20170051068) | YFWGWIRQPPGKGLEWIGSIYYSGITYYNPSLK |
| | | SRVTISVDTSKNQFSLKLSSVTAADTAVYYCAR |
| | | HDGATAGLFDYWGQGTLVTVSS |
| 141 | CDR1 of the heavy chain | SSSYFWG |
| | variable domain of clone 4 | |
| | (US20170051068) | |
| 142 | CDR2 of the heavy chain | SIYYSGITYYNPSLKS |
| | variable domain of clone 4 | |
| | (US20170051068) | |
| 143 | CDR3 of the heavy chain | HDGATAGLFDY |
| | variable domain of clone 4 | |
| | (US20170051068) | |
| 144 | Light chain variable domain of | SYVLTQPPSVSVAPGQTARITCGGNNIGSKSVH |
| | clone 4 (US20170051068) | WYQQPPGQAPVVVVYDDSDRPSGIPERFSGSNS |
| | | GNTATLTISRVEAGDEAVYYCQVWDSSSDHVVF |
| | | GGGTKLTVL |
| 145 | CDR1 of the light chain | GGNNIGSKSVH |
| | variable domain of clone 4 | |
| | (US20170051068) | |
| 146 | CDR2 of the light chain | DDSDRPS |
| | variable domain of clone 4 | |
| | (US20170051068) | |
| 147 | CDR3 of the light chain | QVWDSSSDHVV |
| | variable domain of clone 4 | |
| | (US20170051068) | |
| 148 | Heavy chain variable domain of | EVQLLESGGGLVQPGGSLRLSCAASGFTFSDNA |
| | clone 5 (Mab42 | MGWVRQAPGKGLEWVSAISGPGSSTYYADSVKG |
| | (WO2017021450)) | RFTISRDNSKNTLYLQMNSLRAEDTAVYYCAKV |
| | | LGWFDYWGQGTLVTVSS |
| 149 | CDR1 of the heavy chain | DNAMG |
| | variable domain of clone 5 | |
| | (Mab42 (WO2017021450)) | |
| 150 | CDR2 of the heavy chain | AISGPGSSTYYADSVKG |
| | variable domain of clone 5 | |
| | (Mab42 (WO2017021450)) | |
| 151 | CDR3 of the heavy chain | VLGWFDY |
| | variable domain of clone 5 | |
| | (Mab42 (WO2017021450)) | |
| 152 | Light chain variable domain of | EIVLTQSPGTLSLSPGERATLSCRASQSVSDEY |
| | clone 5 (Mab42 | LSWYQQKPGQAPRLLIHSASTRATGIPDRFSGS |
| | (WO2017021450)) | GSGTDFTLAISRLEPEDFAVYYCQQYGYPPDFT |
| | | FGQGTKVEIK |
| 153 | CDR1 of the light chain | RASQSVSDEYLSW |
| | variable domain of clone 5 | |
| | (Mab42 (WO2017021450)) | |
| 154 | CDR2 of the light chain | HSASTRAT |
| | variable domain of clone 5 | |
| | (Mab42 (WO2017021450)) | |
| 155 | CDR3 of the light chain | QQYGYPPDFT |
| | variable domain of clone 5 | |
| | (Mab42 (WO2017021450)) | |
| 156 | Amino acid sequence can be | MLQMAGQCSQNEYFDSLLHACIPCQLRCSSNTP |
| | used for screening of novel | PLTCQRYCNASVTNSVKGTNAILWTCLGLSLII |
| | antigen-binding sites that can | SLAVEVLMFLLRKINSEPLKDEFKNTGSGLLGM |
| | bind to BCMA | ANIDLEKSRTGDEIILPRGLEYTVEECTCEDCI |
| | | KSKPKVDSDHCFPLPAMEEGATILVTTKTNDYC |
| | | KSLPAALSATEIEKSISAR |
| 157 | 83 A10 heavy chain variable | EVQLLESGGGLVQPGGSLRLSCAASGFTFSSYA |
| | domain | MSWVRQAPGKGLEWVSAISGSGGSTYYADSVKG |
| | | RFTISRDNSKNTLYLQMNSLRAEDTAVYYCAKV |
| | | LGWFDYWGQGTLVTVSS |
| 158 | 83A10 light chain variable | EIVLTQSPGTLSLSPGERATLSCRASQSVSSSY |
| | domain | LAWYQQKPGQAPRLLIYGASSRATGIPDRFSGS |
| | | GSGTDFTLTISRLEPEDFAVYYCQQYGYPPDFT |
| | | FGQGTKVEIK |
| 159 | CDR3 of the heavy chain | GAPNYGDTTHDYYYMDV |
| | variable region of clone ADI- | |
| | 29379 (E79) | |
| 160 | CDR3 of the heavy chain | GAPXGAAAGWFDP, wherein X is M, L, |
| | variable region of clone A49- | I, V, Q, or F |
| | consensus | |
| 161 | NKG2D-binding scFv | DIQMTQSPSSVSASVGDRVTITCRASQGISSWL |
| | | AWYQQKPGKAPKLLIYAASSLQSGVPSRFSGSG |
| | | SGTDFTLTISSLOPEDFATYYCQQGVSFPRTFG |
| | | CGTKVEIKGGGGSGGGGSGGGGSGGGGSEVQLV |
| | | ESGGGLVKPGGSLRLSCAASGFTFSSYSMNWVR |
| | | QAPGKCLEWVSSISSSSSYIYYADSVKGRFTIS |
| | | RDNAKNSLYLQMNSLRAEDTAVYYCARGAPMGA |
| | | AAGWFDPWGQGTLVTVSS |
| 162 | F4-BCMAFc-AJchainB- | EVQLLESGGGLVQPGGSLRLSCAASGFTFSDNA |
| | NKG2D-binding scFv | MGWVRQAPGKGLEWVSAISGPGSSTYYADSVKG |
| | | RFTISRDNSKNTLYLQMNSLRAEDTAVYYCAKV |
| | | LGWFDYWGQGTLVTVSSASTKGPSVFPLAPSSK |
| | | STSGGTAALGCLVKDYFPEPVTVSWNSGALTSG |
| | | VHTFPAVLQSSGLYSLSSVVTVPSSSLGTQTYI |
| | | CNVNHKPSNTKVDKKVEPKSCDKTHTCPPCPAP |
| | | ELLGGPSVFLFPPKPKDTLMISRTPEVTCVVVD |
| | | VSHEDPEVKFNWYVDGVEVHNAKTKPREEQYNS |
| | | TYRVVSVLTVLHQDWLNGKEYKCKVSNKALPAP |
| | | IEKTISKAKGQPREPRVYTLPPCRDELTKNQVS |
| | | LTCLVKGFYPSDIAVEWESNGQPENNYKTTPPV |
| | | LVSDGSFTLYSKLTVDKSRWQQGNVFSCSVMHE |
| | | ALHNHYTOKSLSLSPGSGSGGGGSDIQMTQSPS |
| | | SVSASVGDRVTITCRASQGISSWLAWYQQKPGK |
| | | APKLLIYAASSLQSGVPSRFSGSGSGTDFTLTI |
| | | SSLQPEDFATYYCQQGVSFPRTFGCGTKVEIKG |
| | | GGGSGGGGSGGGGSGGGGSEVQLVESGGGLVKP |
| | | GGSLRLSCAASGFTFSSYSMNWVRQAPGKCLEW |
| | | VSSISSSSSYIYYADSVKGRFTISRDNAKNSLY |
| | | LQMNSLRAEDTAVYYCARGAPMGAAAGWFDPWG |
| | | QGTLVTVSS |
| 163 | Anti-BCMA VH-CH1-Fc | EVQLLESGGGLVQPGGSLRLSCAASGFTFSDNA |
| | | MGWVRQAPGKGLEWVSAISGPGSSTYYADSVKG |
| | | RFTISRDNSKNTLYLQMNSLRAEDTAVYYCAKV |
| | | LGWFDYWGQGTLVTVSSASTKGPSVFPLAPSSK |
| | | STSGGTAALGCLVKDYFPEPVTVSWNSGALTSG |
| | | VHTFPAVLQSSGLYSLSSVVTVPSSSLGTQTYI |
| | | CNVNHKPSNTKVDKKVEPKSCDKTHTCPPCPAP |
| | | ELLGGPSVFLFPPKPKDTLMISRTPEVTCVVVD |
| | | VSHEDPEVKFNWYVDGVEVHNAKTKPREEQYNS |
| | | TYRVVSVLTVLHQDWLNGKEYKCKVSNKALPAP |
| | | IEKTISKAKGQPREPQVCTLPPSRDELTENQVS |
| | | LTCLVKGFYPSDIAVEWESNGQPENNYKTTPPV |
| | | LDSDGSFFLYSWLTVDKSRWQQGNVFSCSVMHE |
| | | ALHNHYTQKSLSLSPG |
| 164 | (G4S)₄ linker | GGGGSGGGGSGGGGSGGGGS |
| 165 | Anti-BCMA-Whole LC | EIVLTQSPGTLSLSPGERATLSCRASQSVSDEY |
| | | LSWYQQKPGQAPRLLIHSASTRATGIPDRESGS |
| | | GSGTDFTLAISRLEPEDFAVYYCQQYGYPPDFT |
| | | FGQGTKVEIKRTVAAPSVFIFPPSDEQLKSGTA |
| | | SVVCLLNNFYPREAKVQWKVDNALQSGNSQESV |
| | | TEQDSKDSTYSLSSTLTLSKADYEKHKVYACEV |
| | | THQGLSSPVTKSFNRGEC |
| 166 | F3- NKG2D-binding scFv-Fc- | DIQMTQSPSSVSASVGDRVTITCRASQGISSWL |
| | AJchainB [V_{L}(G4S)₄V_{H})] | AWYQQKPGKAPKLLIYAASSLQSGVPSRFSGSG |
| | | SGTDFTLTISSLQPEDFATYYCQQGVSFPRTFG |
| | | [symbol] GTKVEIKGGGGSGGGGSGGGGSGGGGSEVQLV |
| | | ESGGGLVKPGGSLRLSCAASGFTFSSYSMNWVR |
| | | QAPGK [symbol] LEWVSSISSSSSYIYYADSVKGRFTIS |
| | | RDNAKNSLYLQMNSLRAEDTAVYYCARGAPMGA |
| | | AAGWFDPWGQGTLVTVSSASDKTHTCPPCPAPE |
| | | LLGGPSVFLFPPKPKDTLMISRTPEVTCVVVDV |
| | | SHEDPEVKFNWYVDGVEVHNAKTKPREEQYNST |
| | | YRVVSVLTVLHQDWLNGKEYKCKVSNKALPAPI |
| | | EKTISKAKGQPREPRVYTLPP [symbol] RDELTKNQVSL |
| | | TCLVKGFYPSDIAVEWESNGQPENNYKTTPPVL |
| | | VSDGSFTLYSKLTVDKSRWQQGNVFSCSVMHEA |
| | | LHNHYTQKSLSLSPG |
| 167 | G₄S linker | GGGGS |
| 168 | SGSGGGGS linker | SGSGGGGS |
| 169 | Heavy chain variable region of | EVQLVESGGGLVKPGGSLRLSCAASGFTFSSYS |
| | clone A49MI | MNWVRQAPGKGLEWVSSISSSSSYIYYADSVKG |
| | | RFTISRDNAKNSLYLQMNSLRAEDTAVYYCARG |
| | | APIGAAAGWFDPWGQGTLVTVSS |
| 170 | CDR3 of the heavy chain | ARGAPIGAAAGWFDP |
| | variable region of clone A49MI | |
| | (non-Kabat) | |
| 171 | Heavy chain variable region of | EVQLVESGGGLVKPGGSLRLSCAASGFTFSSYS |
| | clone A49MQ | MNWVRQAPGKGLEWVSSISSSSSYIYYADSVKG |
| | | RFTISRDNAKNSLYLQMNSLRAEDTAVYYCARG |
| | | APQGAAAGWFDPWGQGTLVTVSS |
| 172 | CDR3 of the heavy chain | ARGAPQGAAAGWFDP |
| | variable region of clone | |
| | A49MQ (non-Kabat) | |
| 173 | Heavy chain variable region of | EVQLVESGGGLVKPGGSLRLSCAASGFTFSSYS |
| | clone A49ML | MNWVRQAPGKGLEWVSSISSSSSYIYYADSVKG |
| | | RFTISRDNAKNSLYLQMNSLRAEDTAVYYCARG |
| | | APLGAAAGWFDPWGQGTLVTVSS |
| 174 | CDR3 of the heavy chain | ARGAPLGAAAGWFDP |
| | variable region of clone | |
| | A49ML (non-Kabat) | |
| 175 | Heavy chain variable region of | EVQLVESGGGLVKPGGSLRLSCAASGFTFSSYS |
| | clone A49MF | MNWVRQAPGKGLEWVSSISSSSSYIYYADSVKG |
| | | RFTISRDNAKNSLYLQMNSLRAEDTAVYYCARG |
| | | APFGAAAGWFDPWGQGTLVTVSS |
| 176 | CDR3 of the heavy chain | ARGAPFGAAAGWFDP |
| | variable region of clone A49MF | |
| | (non-Kabat) | |
| 177 | Heavy chain variable region of | EVQLVESGGGLVKPGGSLRLSCAASGFTFSSYS |
| | clone A49MV | MNWVRQAPGKGLEWVSSISSSSSYIYYADSVKG |
| | | RFTISRDNAKNSLYLQMNSLRAEDTAVYYCARG |
| | | APVGAAAGWFDPWGQGTLVTVSS |
| 178 | CDR3 of the heavy chain | ARGAPVGAAAGWFDP |
| | variable region of clone | |
| | A49MV (non-Kabat) | |
| 179 | Heavy chain variable region of | EVQLVESGGGLVKPGGSLRLSCAASGFTFSSYS |
| | clone A49-consensus | MNWVRQAPGKGLEWVSSISSSSSYIYYADSVKG |
| | | RFTISRDNAKNSLYLQMNSLRAEDTAVYYCARG |
| | | APXGAAAGWFDPWGQGTLVTVSS, wherein |
| | | X is M, L, I, V, Q, or F |
| 180 | CDR3 of the heavy chain | ARGAPXGAAAGWFDP, wherein X is M, |
| | variable region of clone A49- | L, I, V, Q, or F |
| | consensus (non-Kabat) | |
| 181 | CDR1 of the heavy chain | SYAIS |
| | variable region of clone ADI- | |
| | 28200 | |
| 182 | CDR3 of the heavy chain | GDSSIRHAYYYYGMDV |
| | variable region of clone ADI- | |
| | 27727 | |
| 183 | CDR1 of the heavy chain | SSSYYWG |
| | variable region of clone ADI- | |
| | 24443 (F43) | |
| 184 | CDR3 of the heavy chain | GSDRFHPYFDY |
| | variable region of clone ADI- | |
| | 24443 (F43) | |
| 185 | CDR1 of the heavy chain | SYYMH |
| | variable region of clone ADI- | |
| | 29379 (E79) or ADI-29378 | |
| | (E78) | |
| 186 | CDR1 of the heavy chain | GYYMH |
| | variable region of clone ADI- | |
| | 29463 (F63) | |
| 187 | CDR3 of the heavy chain | DTGEYYDTDDHGMDV |
| | variable region of clone ADI- | |
| | 29463 (F63) | |
| 188 | CDR1 of the heavy chain | SYAMS |
| | variable region of clone ADI- | |
| | 27744 (A44) | |
| 189 | CDR3 of the heavy chain | DGGYYDSGAGDY |
| | variable region of clone ADI- | |
| | 27744 (A44) | |
| 190 | CDR1 of the heavy chain | SYSMN |
| | variable region of clone | |
| | AA49MI, 49MQ, A49ML, A49MF, | |
| | A49MV, or A49-consensus | |
| 191 | CDR3 of the heavy chain | GAPMGAAAGWFDP |
| | variable region of clone ADI- | |
| | 27749 (A49) | |
| 192 | CDR3 of the heavy chain | EGAGFAYGMDYYYMDV |
| | variable region of clone ADI- | |
| | 29378 (E78) | |
| 193 | CDR3 of the heavy chain | GAPIGAAAGWFDP |
| | variable region of clone A49MI | |
| 194 | CDR3 of the heavy chain | GAPQGAAAGWFDP |
| | variable region of clone | |
| | A49MQ | |
| 195 | CDR3 of the heavy chain | GAPLGAAAGWFDP |
| | variable region of clone | |
| | A49ML | |
| 196 | CDR3 of the heavy chain | GAPFGAAAGWFDP |
| | variable region of clone A49MF | |
| 197 | CDR3 of the heavy chain | GAPVGAAAGWFDP |
| | variable region of clone | |
| | A49MV | |
| 198 | (G4S)ₙ linker | (GlyGlyGlyGlySer)ₙ, wherein n is |
| | | an integer between 1-10 |

What is claimed:

1. A method of treating, managing and/or preventing cancer, comprising administering to a subject in need of such treatment, management and/or prevention a therapeutically or prophylactically effective combination of:
(a) a protein that comprises (i) an antigen-binding site that binds an antigen on a natural killer (NK) cell, and (ii) an antigen-binding site that binds an antigen on a cell of said cancer; and
(b) iberdomide (CC-220), avadomide (CC-122), or lenalidomide.

2. A method of treating, managing and/or preventing cancer, comprising administering to a subject in need of such treatment, management and/or prevention a therapeutically or prophylactically effective combination of:
(a) a protein that comprises (i) an antigen-binding site that binds an antigen on a natural killer (NK) cell, and (ii) an antigen-binding site that binds an antigen on a cell of said cancer; and
(b) iberdomide (CC-220), avadomide (CC-122), lenalidomide, or pomalidomide, and
wherein the method does not comprise administering IL-2.

3. A method of treating, managing and/or preventing cancer, comprising administering to a subject in need of such treatment, management and/or prevention a therapeutically or prophylactically effective combination of:
(a) a protein that comprises (i) an antigen-binding site that binds NKG2D, and (ii) an antigen-binding site that binds an antigen on a cell of said cancer; and
(b) iberdomide (CC-220), avadomide (CC-122), or lenalidomide.

4. A method of treating, managing and/or preventing cancer, comprising administering to a subject in need of such treatment, management and/or prevention a therapeutically or prophylactically effective combination of:
(a) a protein that comprises (i) an antigen-binding site that binds NKG2D, and (ii) an antigen-binding site that binds an antigen on a cell of said cancer; and
(b) iberdomide (CC-220), avadomide (CC-122), lenalidomide, or pomalidomide, and
wherein the method does not comprise administering IL-2.

5. The method of any of claims 1 to 2, wherein said antigen-binding site that binds to an antigen on an NK cell binds NKG2D.

6. The method of any of claims 1 to 5, wherein said protein and said iberdomide (CC-220), avadomide (CC-122), lenalidomide, or pomalidomide activate NK cells to a greater degree than either of said protein or said iberdomide (CC-220), avadomide (CC-122), lenalidomide, or pomalidomide, alone.

7. The method of any of claims 1 to 6, wherein said antigen on said cancer cell is BCMA.

8. The method of any one of claims 1 to 7, wherein said protein further comprises an antibody Fc domain capable of binding CD16, a portion of an antibody Fc domain capable of binding CD16, or an antigen-binding site that binds CD16.

9. The method of any one of claims 3 to 8, wherein said antigen-binding site that binds NKG2D comprises a single-chain variable fragment (scFv) that binds NKG2D.

10. The method of claim 9, wherein said scFv that binds to NKG2D is linked to said antibody Fc domain capable of binding CD16, said portion of an antibody Fc domain capable of binding CD16, or said antigen-binding site that binds CD16.

11. The method of any one of claims 3 to 8, wherein said antigen-binding site that binds NKG2D comprises a Fab that binds NKG2D.

12. The method of claim 11, wherein said Fab that binds to NKG2D is linked to said antibody Fc domain capable of binding CD16, said portion of an antibody Fc domain capable of binding CD16, or said antigen-binding site that binds CD16.

13. The method of any one of claims 3 to 12, wherein said antigen-binding site that binds NKG2D comprises:
(a) a heavy chain variable domain comprising complementarity-determining region 1 (CDR1) that comprises the amino acid sequence of SEQ ID NO:190, complementarity-determining region 2 (CDR2) that comprises the amino acid sequence of SEQ ID NO:96, and complementarity-determining region 3 (CDR3) that comprised the amino acid sequence of SEQ ID NO:191; and
(b) a light chain variable domain comprising CDR1 that comprises the amino acid sequence of SEQ ID NO:99, CDR2 that comprises the amino acid sequence of SEQ ID NO:100, and CDR3 that comprises the amino acid sequences of SEQ ID NO:101.

14. The method of any one of claims 3 to 13, wherein the antigen-binding site that binds NKG2D comprises
(a) a heavy chain variable domain at least 90% identical to SEQ ID NO:94 and a light chain variable domain at least 90% identical to SEQ ID NO:98;
(b) a heavy chain variable domain at least 95% identical to SEQ ID NO:94 and a light chain variable domain at least 95% identical to SEQ ID NO:98; or
(c) a heavy chain variable domain identical to SEQ ID NO:94 and a light chain variable domain identical to SEQ ID NO:98.

15. The method of any one of claims 7 to 14, wherein said antigen-binding site that binds BCMA comprises:
(a) a heavy chain variable domain comprising CDR1 that comprises the amino acid sequence of SEQ ID NO:149, CDR2 that comprises the amino acid sequence of SEQ ID NO:150, and CDR3 that comprises the amino acid sequence of SEQ ID NO:151, and
(b) a light chain variable domain comprising CDR1 that comprises the amino acid sequence of SEQ ID NO:153, CDR2 that comprises the amino acid sequence of SEQ ID NO:154, and CDR3 that comprises the amino acid sequence of SEQ ID NO:155.

16. The method of any one of claims 7 to 15, wherein said antigen-binding site that binds BCMA comprises:
(a) a heavy chain variable domain at least 90% identical to SEQ ID NO:148 and a light chain variable domain at least 90% identical to SEQ ID NO:152;
(b) a heavy chain variable domain at least 95% identical to SEQ ID NO:148 and a light chain variable domain at least 95% identical to SEQ ID NO:152; or
(c) a heavy chain variable domain identical to SEQ ID NO:148 and a light chain variable domain identical to SEQ ID NO:152.

17. The method of any one of claims 7 to 16, wherein said protein further comprises an additional antigen-binding site that binds said antigen on a cell of said cancer.

18. The method of claim 17, wherein said additional antigen-binding site binds BCMA.

19. The method of claim 18, wherein said additional antigen-binding site that binds BCMA comprises:
(a) a heavy chain variable domain comprising CDR1 that comprises the amino acid sequence of SEQ ID NOs:149, CDR2 that comprises the amino acid sequence of SEQ ID NO:150, and CDR3 that comprises the amino acid sequence of SEQ ID NOs:151, and
(b) a light chain variable domain comprising CDR1 that comprises the amino acid sequence of SEQ ID NOs:153, CDR2 that comprises the amino acid sequence of SEQ ID NO:154, and CDR3 that comprises the amino acid sequence of SEQ ID NOs:155.

20. The method of any one of claims 18 to 19, wherein said additional antigen-binding site that binds BCMA comprises:
(a) a heavy chain variable domain at least 90% identical to SEQ ID NO:148 and a light chain variable domain at least 90% identical to SEQ ID NO:152;
(b) a heavy chain variable domain at least 95% identical to SEQ ID NO:148 and a light chain variable domain at least 95% identical to SEQ ID NO:152; or
(c) a heavy chain variable domain identical to SEQ ID NO:148 and a light chain variable domain identical to SEQ ID NO:152.

21. The method of any one of claims 1 to 20, wherein said protein comprises an antibody Fc domain.

22. The method of claim 21, wherein said antibody Fc domain comprises hinge and CH2 domains of a human IgG1 antibody.

23. The method of any one of claims 21 to 22, wherein said Fc domain comprises an amino acid sequence at least 90% identical to amino acids 234-332 of a human IgG1 antibody.

24. The method of any one of claims 1 to 23, wherein said protein comprises a polypeptide comprising the amino acid sequence of SEQ ID NO:161.

25. The method of any one of claims 1 to 24, wherein said protein comprises a polypeptide comprising an amino acid sequence at least 90%, at least 95% or at least 99% identical to the amino acid sequence of SEQ ID NO:162.

26. The method of claim 25, wherein said protein comprises (i) a polypeptide comprising the amino acid sequence of SEQ ID NO:162.

27. The method of claim 26, wherein said protein further comprises (ii) a polypeptide comprising the amino acid sequence of SEQ ID NO:163; and (iii) a polypeptide comprising the amino acid sequence of SEQ ID NO:165.

28. The method of claim 26, wherein said protein further comprises (ii) a polypeptide comprising the amino acid sequence of SEQ ID NO:163; and (iii) two polypeptides each comprising the amino acid sequence of SEQ ID NO:165.

29. The method of claim 28, wherein said polypeptides are linked as represented in FIG. 15.

30. The method of claim 28, wherein said polypeptide comprising the amino acid sequence of SEQ ID NO:162 is linked to the polypeptide comprising the amino acid sequence of SEQ ID NO:163 via heterodimerization and at least one disulfide bond; wherein the polypeptide comprising the amino acid sequence of SEQ ID NO:165 is linked to the polypeptide comprising the amino acid sequence of SEQ ID NO:162 via a disulfide bond; and wherein the other polypeptide comprising the amino acid sequence of SEQ ID NO:165 is linked to the polypeptide comprising the amino acid sequence of SEQ ID NO:163 via a disulfide bond.

31. A method of treating, managing and/or preventing cancer, comprising administering to a subject in need of such treatment, management and/or prevention a therapeutically or prophylactically effective combination of:
(a) a protein that comprises (i) a polypeptide comprising the amino acid sequence of SEQ ID NO:162; (ii) a polypeptide comprising the amino acid sequence of SEQ ID NO:163; and (iii) two polypeptides each comprising the amino acid sequence of SEQ ID NO:165, and
(b) iberdomide (CC-220), avadomide (CC-122), lenalidomide, or pomalidomide,
wherein the polypeptide comprising the amino acid sequence of SEQ ID NO:162 is linked to the polypeptide comprising the amino acid sequence of SEQ ID NO:163 via heterodimerization and at least one disulfide bond; wherein the polypeptide comprising the amino acid sequence of SEQ ID NO:165 is linked to the polypeptide comprising the amino acid sequence of SEQ ID NO:162 via a disulfide bond; and wherein the other polypeptide comprising the amino acid sequence of SEQ ID NO:165 is linked to the polypeptide comprising the amino acid sequence of SEQ ID NO:163 via a disulfide bond.

32. The method of any one of claims 1 to 31, wherein said cancer is leukemia, myeloma, non-Hodgkin's lymphoma (NHL), Hodgkin's lymphoma (HL), and Waldenstrom's macroglobulinemia.

33. The method of claim 32, wherein said cancer is acute lymphoblastic leukemia (ALL), acute myeloid leukemia (AML), chronic lymphocytic leukemia/small lymphocytic lymphoma (CLL/SLL), chronic myeloid leukemia (CML), chronic myelomonocytic leukemia (CMML), multiple myeloma (MM), classic Hodgkin lymphoma (cHL), nodular lymphocyte-predominant Hodgkin lymphoma, follicular lymphoma (FL), lymphoplasmacytic lymphoma, marginal zone lymphoma (MZL), primary cutaneous anaplastic large cell lymphoma, diffuse large B-cell lymphoma (DLBCL), follicular large cell lymphoma, anaplastic large cell lymphoma, extranodal NK-/T-cell lymphoma, lymphomatoid granulomatosis, angioimmunoblastic T-cell lymphoma, peripheral T-cell lymphoma (PTCL), primary central nervous system lymphoma (PCNSL), intravascular large B-cell lymphoma, mantle cell lymphoma (MCL), post-transplantation lymphoproliferative disorder, Burkitt lymphoma, lymphoblastic lymphoma, adult T-cell leukemia/lymphoma, B-cell lymphomas, glioblastoma, true histiocytic lymphoma, primary effusion lymphoma, or plasmablastic lymphoma.

34. The method of any one of claims 1 to 33, wherein said cancer is relapsed or refractory.

35. The method of any one of claims 1 to 33, wherein said cancer is newly diagnosed.

36. The method of any one of claims 1 to 33, wherein said subject has failed at least one prior therapy.

37. The method of any one of claims 1 to 36, wherein said subject is less than 18 years old.

38. The method of any one of claims 1 to 36, wherein said subject is 18 years old or older.

39. The method of any one of claims 1 to 38, wherein the combination of (a) said protein and (b) said iberdomide (CC-220), avadomide (CC-122), lenalidomide, or pomalidomide is in a therapeutically or prophylactically effective amount.

40. The method of any one of claims 1 to 38, wherein said protein is in a therapeutically or prophylactically effective amount.

41. The method of any one of claims 1 to 38, wherein said iberdomide (CC-220), avadomide (CC-122), lenalidomide, or pomalidomide is in a therapeutically or prophylactically effective amount.

42. The method of any one of claims 1 to 41, wherein said method comprises administering iberdomide (CC-220).

43. The method of claim 42, wherein said iberdomide is administered in an amount of about 0.1 mg to about 2 mg per day.

44. The method of any one of claims 1 to 41, wherein said method comprises administering avadomide (CC-122).

45. The method of claim 44, wherein said avadomide is administered in an amount of about 2 mg to about 50 mg per day.

46. The method of any one of claims 1 to 41, wherein said method comprises administering lenalidomide.

47. The method of claim 46, wherein said lenalidomide is administered in an amount of about 10 mg to about 25 mg per day.

48. The method of any one of claims 1 to 41, wherein said method comprises administering pomalidomide.

49. The method of claim 48, wherein said pomalidomide is administered in an amount of about 3 mg to about 5 mg per day.

50. The method of any one of claims 1 to 49, wherein said iberdomide, avadomide, lenalidomide, or pomalidomide is administered on days 1 to 5 of a 7-day cycle or days 1 to 21 of a 28-day cycle.

51. The method of any one of claims 1 to 50, wherein said method further comprises administering a third agent that is or comprises dexamethasone, iberdomide, avadomide, lenalidomide, or pomalidomide.

52. A method of treating, managing and/or preventing cancer, comprising administering to a subject in need of such treatment, management and/or prevention a therapeutically or prophylactically effective combination of:
(a) a protein that comprises (i) an antigen-binding site that binds an antigen on a natural killer (NK) cell, and (ii) an antigen-binding site that binds an antigen on a cell of said cancer; and
(b) CC-92480.

53. A method of treating, managing and/or preventing cancer, comprising administering to a subject in need of such treatment, management and/or prevention a therapeutically or prophylactically effective combination of:
(a) a protein that comprises (i) an antigen-binding site that binds NKG2D, and (ii) an antigen-binding site that binds an antigen on a cell of said cancer; and
(b) CC-92480.

54. The method of claim 52, wherein said antigen-binding site that binds to an antigen on an NK cell binds NKG2D.

55. The method of any of claims 52 to 54, wherein said method does not comprise administering IL-2.

56. The method of any of claims 52 to 54, wherein said antigen on said cancer cell is BCMA.

57. The method of any one of claims 52 to 56, wherein said protein further comprises an antibody Fc domain capable of binding CD16, a portion of an antibody Fc domain capable of binding CD16, or an antigen-binding site that binds CD16.

58. The method of any one of claims 53 to 57, wherein said antigen-binding site that binds NKG2D comprises a single-chain variable fragment (scFv) that binds NKG2D.

59. The method of claim 58, wherein said scFv that binds to NKG2D is linked to said antibody Fc domain capable of binding CD16, said portion of an antibody Fc domain capable of binding CD16, or said antigen-binding site that binds CD16.

60. The method of any one of claims 53 to 59, wherein said antigen-binding site that binds NKG2D comprises:
(a) a heavy chain variable domain comprising complementarity-determining region 1 (CDR1) that comprises the amino acid sequence of SEQ ID NO:190, complementarity-determining region 2 (CDR2) that comprises the amino acid sequence of SEQ ID NO:96, and complementarity-determining region 3 (CDR3) that comprised the amino acid sequence of SEQ ID NO:191; and
(b) a light chain variable domain comprising CDR1 that comprises the amino acid sequence of SEQ ID NO:99, CDR2 that comprises the amino acid sequence of SEQ ID NO:100, and CDR3 that comprises the amino acid sequences of SEQ ID NO:101.

61. The method of any one of claims 53 to 60, wherein the antigen-binding site that binds NKG2D comprises
(a) a heavy chain variable domain at least 90% identical to SEQ ID NO:94 and a light chain variable domain at least 90% identical to SEQ ID NO:98;
(b) a heavy chain variable domain at least 95% identical to SEQ ID NO:94 and a light chain variable domain at least 95% identical to SEQ ID NO:98; or
(c) a heavy chain variable domain identical to SEQ ID NO:94 and a light chain variable domain identical to SEQ ID NO:98.

62. The method of any one of claims 56 to 61, wherein said antigen-binding site that binds BCMA comprises:
(a) a heavy chain variable domain comprising CDR1 that comprises the amino acid sequence of SEQ ID NO:149, CDR2 that comprises the amino acid sequence of SEQ ID NO:150, and CDR3 that comprises the amino acid sequence of SEQ ID NO:151, and
(b) a light chain variable domain comprising CDR1 that comprises the amino acid sequence of SEQ ID NO:153, CDR2 that comprises the amino acid sequence of SEQ ID NO:154, and CDR3 that comprises the amino acid sequence of SEQ ID NO:155.

63. The method of any one of claims 56 to 62, wherein said antigen-binding site that binds BCMA comprises:
(a) a heavy chain variable domain at least 90% identical to SEQ ID NO:148 and a light chain variable domain at least 90% identical to SEQ ID NO:152;
(b) a heavy chain variable domain at least 95% identical to SEQ ID NO:148 and a light chain variable domain at least 95% identical to SEQ ID NO:152; or
(c) a heavy chain variable domain identical to SEQ ID NO:148 and a light chain variable domain identical to SEQ ID NO:152.

64. The method of any one of claims 56 to 63, wherein said protein further comprises an additional antigen-binding site that binds BCMA.

65. The method of claim 64, wherein said additional antigen-binding site that binds BCMA comprises:
(a) a heavy chain variable domain comprising CDR1 that comprises the amino acid sequence of SEQ ID NOs:149, CDR2 that comprises the amino acid sequence of SEQ ID NO:150, and CDR3 that comprises the amino acid sequence of SEQ ID NOs:151, and
(b) a light chain variable domain comprising CDR1 that comprises the amino acid sequence of SEQ ID NOs:153, CDR2 that comprises the amino acid sequence of SEQ ID NO:154, and CDR3 that comprises the amino acid sequence of SEQ ID NOs:155.

66. The method of any one of claims 64 to 65, wherein said additional antigen-binding site that binds BCMA comprises:
(a) a heavy chain variable domain at least 90% identical to SEQ ID NO:148 and a light chain variable domain at least 90% identical to SEQ ID NO:152;
(b) a heavy chain variable domain at least 95% identical to SEQ ID NO:148 and a light chain variable domain at least 95% identical to SEQ ID NO:152; or
(c) a heavy chain variable domain identical to SEQ ID NO:148 and a light chain variable domain identical to SEQ ID NO:152.

67. The method of any one of claims 52 to 66, wherein said protein comprises an antibody Fc domain, wherein said Fc domain comprises an amino acid sequence at least 90% identical to amino acids 234-332 of a human IgG1 antibody.

68. The method of any one of claims 52 to 67, wherein said protein comprises (i) a polypeptide comprising the amino acid sequence of SEQ ID NO:162; (ii) a polypeptide comprising the amino acid sequence of SEQ ID NO:163; and (iii) two polypeptides each comprising the amino acid sequence of SEQ ID NO:165.

69. The method of claim 68, wherein said polypeptides are linked as represented in FIG. 15.

70. The method of claim 68, wherein said polypeptide comprising the amino acid sequence of SEQ ID NO:162 is linked to the polypeptide comprising the amino acid sequence of SEQ ID NO:163 via heterodimerization and at least one disulfide bond; wherein the polypeptide comprising the amino acid sequence of SEQ ID NO:165 is linked to the polypeptide comprising the amino acid sequence of SEQ ID NO:162 via a disulfide bond; and wherein the other polypeptide comprising the amino acid sequence of SEQ ID NO:165 is linked to the polypeptide comprising the amino acid sequence of SEQ ID NO:163 via a disulfide bond.

71. A method of treating, managing and/or preventing cancer, comprising administering to a subject in need of such treatment, management and/or prevention a therapeutically or prophylactically effective combination of:
(a) a protein that comprises (i) a polypeptide comprising the amino acid sequence of SEQ ID NO:162; (ii) a polypeptide comprising the amino acid sequence of SEQ ID NO:163; and (iii) two polypeptides each comprising the amino acid sequence of SEQ ID NO:165, and
(b) CC-92480,
wherein the polypeptide comprising the amino acid sequence of SEQ ID NO:162 is linked to the polypeptide comprising the amino acid sequence of SEQ ID NO:163 via heterodimerization and at least one disulfide bond; wherein the polypeptide comprising the amino acid sequence of SEQ ID NO:165 is linked to the polypeptide comprising the amino acid sequence of SEQ ID NO:162 via a disulfide bond; and wherein the other polypeptide comprising the amino acid sequence of SEQ ID NO:165 is linked to the polypeptide comprising the amino acid sequence of SEQ ID NO:163 via a disulfide bond.

72. The method of any one of claims 52 to 71, wherein said cancer is leukemia, myeloma, non-Hodgkin's lymphoma (NHL), Hodgkin's lymphoma (HL), and Waldenstrom's macroglobulinemia.

73. The method of claim 72, wherein said cancer is acute lymphoblastic leukemia (ALL), acute myeloid leukemia (AML), chronic lymphocytic leukemia/small lymphocytic lymphoma (CLL/SLL), chronic myeloid leukemia (CML), chronic myelomonocytic leukemia (CMML), multiple myeloma (MM), classic Hodgkin lymphoma (cHL), nodular lymphocyte-predominant Hodgkin lymphoma, follicular lymphoma (FL), lymphoplasmacytic lymphoma, marginal zone lymphoma (MZL), primary cutaneous anaplastic large cell lymphoma, diffuse large B-cell lymphoma (DLBCL), follicular large cell lymphoma, anaplastic large cell lymphoma, extranodal NK-/T-cell lymphoma, lymphomatoid granulomatosis, angioimmunoblastic T-cell lymphoma, peripheral T-cell lymphoma (PTCL), primary central nervous system lymphoma (PCNSL), intravascular large B-cell lymphoma, mantle cell lymphoma (MCL), post-transplantation lymphoproliferative disorder, Burkitt lymphoma, lymphoblastic lymphoma, adult T-cell leukemia/lymphoma, B-cell lymphomas, glioblastoma, true histiocytic lymphoma, primary effusion lymphoma, or plasmablastic lymphoma.

74. The method of any one of claims 52 to 73, wherein said cancer is relapsed or refractory.

75. The method of any one of claims 52 to 74, wherein said CC-92480 is administered on days 1 to 5 of a 7-day cycle or days 1 to 21 of a 28-day cycle.

76. The method of claims 52 to 75, wherein said CC-92480 is administered in an amount of about 0.1 mg to about 2 mg per day.

77. The method of any one of claims 52 to 76, wherein said method further comprises administering a third agent that is or comprises dexamethasone, iberdomide, avadomide, lenalidomide, or pomalidomide.
